SEQUENCE LISTING (78 sequences)

SEQ ID NO: 1 - protein, 1277 aa, Mus musculus
PGVTVLKMPEPGKKPVSAFNKKPRSAEVTAGSAAVFEAETERSGVKVRWQRDGSDITANDKYGLAAEGKRHTLTVRDASPDDQGSYAVIAGSSKVKFDLKVTEPAPPEKAESEVAPGAPKEVPAPATELEESVSSPEGSVSVTQDGSAAEHQGAPDDPIGLFLMRPQDGEVTVGGSIVFSARVAGASLLKPPVVKWFKGKWVDLSSKVGQHLQLHDSYDRASKVYLFELHITDAQTTSAGGYRCEVSTKDKFDSCNFNLTVHEAIGSGDLDLRSAFRRTSLAGAGRRTSDSHEDAGTLDFSSLLKKRDSFRRDSKLEAPAEEDVWEILRQAPPSEYERIAFQHGVTDLRGMLKRLKGMKQDEKKSTAFQKKLEPAYQVNKGHKIRLTVELADPDAEVKWLKNGQEIQMSGSKYIFESVGAKRTLTISQCSLADDAAYQCVVGGEKCSTELFVKEPPVLITRSLEDQLVMVGQRVEFECEVSEEGAQVKWLKDGVELTREETFKYRFKKDGRKHHLIINEATLEDAGHYAVRTSGGQSLAELIVQEKKLEVYQSIADLAVGAKDQAVFKCEVSDENVRGVWLKNGKELVPDNRIKVSHIGRVHKLTIDDVTPADEADYSFVPEGFACNLSAKLHFMEVKIDFVPRQEPPKIHLDCPGSTPDTIVVVAGNKLRLDVPISGDPAPTVVWQKTVTQGKKASTGPHPDAPEDAGADEEWVFDKKLLCETEGRVRVETTKDRSVFTVEGAEKEDEGVYTVTVKNPVGEDQVNLTVKVIDVPDAPAAPKISNVGEDSCTVQWEPPAYDGGQPVLGYILERKKKKSYRWMRLNFDLLRELSHEARRMIEGVAYEMRVYAVNAVGMSRPSPASQPFMPIGPPGEPTHLAVEDVSDTTVSLKWRPPERVGAGGLDGYSVEYCQEGCSEWTPALQGLTERTSMLVKDLPTGARLLFRVRAHNVAGPGGPIVTKEPVTVQEILQRPRLQLPRHLRQTIQKKVGEPVNLLIPFQGKPRPQVTWTKEGQPLAGEEVSIRNSPTDTILFIRAARRTHSGTYQVTVRIENMEDKATLILQIVDKPSPPQDIRIVETWGFNVALEWKPPQDDGNTEIWGYTVQKADKKTMEWFTVLEHYRRTHCVVSELIIGNGYYFRVFSHNMVGSSDKAAATKEPVFIPRPGITYEPPKYKALDFSEAPSFTQPLANRSIIAGYNAILCCAVRGSPKPKISWFKNGLDLGEDARFRMFCKQGVLTLEIRKPCPYDGGVYVCRATNLQGEAQCECRLEVRVPQ

SEQ ID NO: 2 - protein, 194 aa, Mus musculus
APAAPKISNVGEDSCTVQWEPPAYDGGQPVLGYILERKKKKSYRWMRLNFDLLRELSHEARRMIEGVAYEMRVYAVNAVGMSRPSPASQPFMPIGPPGEPTHLAVEDVSDTTVSLKWRPPERVGAGGLDGYSVEYCQEGCSEWTPALQGLTERTSMLVKDLPTGARLLFRVRAHNVAGPGGPIVTKEPVTVQEI

SEQ ID NO: 3 - protein, 292 aa, Mus musculus
APAAPKISNVGEDSCTVQWEPPAYDGGQPVLGYILERKKKKSYRWMRLNFDLLRELSHEARRMIEGVAYEMRVYAVNAVGMSRPSPASQPFMPIGPPGEPTHLAVEDVSDTTVSLKWRPPERVGAGGLDGYSVEYCQEGCSEWTPALQGLTERTSMLVKDLPTGARLLFRVRAHNVAGPGGPIVTKEPVTVQEILQRPRLQLPRHLRQTIQKKVGEPVNLLIPFQGKPRPQVTWTKEGQPLAGEEVSIRNSPTDTILFIRAARRTHSGTYQVTVRIENMEDKATLILQIVDK

SEQ ID NO: 4 - protein, 390 aa, Mus musculus
APAAPKISNVGEDSCTVQWEPPAYDGGQPVLGYILERKKKKSYRWMRLNFDLLRELSHEARRMIEGVAYEMRVYAVNAVGMSRPSPASQPFMPIGPPGEPTHLAVEDVSDTTVSLKWRPPERVGAGGLDGYSVEYCQEGCSEWTPALQGLTERTSMLVKDLPTGARLLFRVRAHNVAGPGGPIVTKEPVTVQEILQRPRLQLPRHLRQTIQKKVGEPVNLLIPFQGKPRPQVTWTKEGQPLAGEEVSIRNSPTDTILFIRAARRTHSGTYQVTVRIENMEDKATLILQIVDKPSPPQDIRIVETWGFNVALEWKPPQDDGNTEIWGYTVQKADKKTMEWFTVLEHYRRTHCVVSELIIGNGYYFRVFSHNMVGSSDKAAATKEPVFIPRP

SEQ ID NO: 5 - protein, 501 aa, Mus musculus
APAAPKISNVGEDSCTVQWEPPAYDGGQPVLGYILERKKKKSYRWMRLNFDLLRELSHEARRMIEGVAYEMRVYAVNAVGMSRPSPASQPFMPIGPPGEPTHLAVEDVSDTTVSLKWRPPERVGAGGLDGYSVEYCQEGCSEWTPALQGLTERTSMLVKDLPTGARLLFRVRAHNVAGPGGPIVTKEPVTVQEILQRPRLQLPRHLRQTIQKKVGEPVNLLIPFQGKPRPQVTWTKEGQPLAGEEVSIRNSPTDTILFIRAARRTHSGTYQVTVRIENMEDKATLILQIVDKPSPPQDIRIVETWGFNVALEWKPPQDDGNTEIWGYTVQKADKKTMEWFTVLEHYRRTHCVVSELIIGNGYYFRVFSHNMVGSSDKAAATKEPVFIPRPGITYEPPKYKALDFSEAPSFTQPLANRSIIAGYNAILCCAVRGSPKPKISWFKNGLDLGEDARFRMFCKQGVLTLEIRKPCPYDGGVYVCRATNLQGEAQCECRLEVRVPQ

SEQ ID NO: 6 - protein, 304 aa, Mus musculus
PRLQLPRHLRQTIQKKVGEPVNLLIPFQGKPRPQVTWTKEGQPLAGEEVSIRNSPTDTILFIRAARRTHSGTYQVTVRIENMEDKATLILQIVDKPSPPQDIRIVETWGFNVALEWKPPQDDGNTEIWGYTVQKADKKTMEWFTVLEHYRRTHCVVSELIIGNGYYFRVFSHNMVGSSDKAAATKEPVFIPRPGITYEPPKYKALDFSEAPSFTQPLANRSIIAGYNAILCCAVRGSPKPKISWFKNGLDLGEDARFRMFCKQGVLTLEIRKPCPYDGGVYVCRATNLQGEAQCECRLEVRVPQ

SEQ ID NO: 7 - protein, 207 aa, Mus musculus
PPQDIRIVETWGFNVALEWKPPQDDGNTEIWGYTVQKADKKTMEWFTVLEHYRRTHCVVSELIIGNGYYFRVFSHNMVGSSDKAAATKEPVFIPRPGITYEPPKYKALDFSEAPSFTQPLANRSIIAGYNAILCCAVRGSPKPKISWFKNGLDLGEDARFRMFCKQGVLTLEIRKPCPYDGGVYVCRATNLQGEAQCECRLEVRVPQ

SEQ ID NO: 8 - protein, 94 aa, Mus musculus
PSFTQPLANRSIIAGYNAILCCAVRGSPKPKISWFKNGLDLGEDARFRMFCKQGVLTLEIRKPCPYDGGVYVCRATNLQGEAQCECRLEVRVPQ

SEQ ID NO: 9 - protein, 1273 aa, Homo sapiens
PEPGKKPVSAFSKKPRSVEVAAGSPAVFEAETERAGVKVRWQRGGSDISASNKYGLATEGTRHTLTVREVGPADQGSYAVIAGSSKVKFDLKVIEAEKAEPMLAPAPAPAEATGAPGEAPAPAAELGESAPSPKGSSSAALNGPTPGAPDDPIGLFVMRPQDGEVTVGGSITFSARVAGASLLKPPVVKWFKGKWVDLSSKVGQHLQLHDSYDRASKVYLFELHITDAQPAFTGSYRCEVSTKDKFDCSNFNLTVHEAMGTGDLDLLSAFRRTSLAGGGRRISDSHEDTGILDFSSLLKKRDSFRTPRDSKLEAPAEEDVWEILRQAPPSEYERIAFQYGVTDLRGMLKRLKGMRRDEKKSTAFQKKLEPAYQVSKGHKIRLTVELADHDAEVKWLKNGQEIQMSGSKYIFESIGAKRTLTISQCSLADDAAYQCVVGGEKCSTELFVKEPPVLITRPLEDQLVMVGQRVEFECEVSEEGAQVKWLKDGVELTREETFKYRFKKDGQRHHLIINEAMLEDAGHYALCTSGGQALAELIVQEKKLEVYQSIADLMVGAKDQAVFKCEVSDENVRGVWLKNGKELVPDSRIKVSHIGRVHKLTIDDVTPADEADYSFVPEGFACNLSAKLHFMEVKIDFVPRQEPPKIHLDCPGRIPDTIVVVAGNKLRLDVPISGDPAPTVIWQKAITQGNKAPARPAPDAPEDTGDSDEWVFDKKLLCETEGRVRVETTKDRSIFTVEGAEKEDEGVYTVTVKNPVGEDQVNLTVKVIDVPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 10 - protein, 194 aa, Homo sapiens
APAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEI

SEQ ID NO: 11 - protein, 292 aa, Homo sapiens
APAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDK

SEQ ID NO: 12 - protein, 390 aa, Homo sapiens
APAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRP

SEQ ID NO: 13 - protein, 501 aa, Homo sapiens
APAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 14 - protein, 304 aa, Homo sapiens
PRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 15 - protein, 207 aa, Homo sapiens
PPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 16 - protein, 94 aa, Homo sapiens
PSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 17 - DNA, 3831 bp, Mus musculus
cctggtgtgactgttctcaagatgccggagccagggaagaaaccagtgtcagccttcaacaagaagccaaggtcagcggaggtgaccgctggcagtgctgccgtgttcgaggctgagacggagcggtcaggcgtgaaggtgcggtggcagcgggatggcagcgacatcaccgccaatgacaagtatggtttggcagcagagggcaagcgacacacactgacagtgcgggatgcgagccctgatgaccagggttcctacgcggtcattgcaggctcctcaaaggtcaagtttgacctcaaggtcacagagccagcccctccagagaaggcagaatctgaagttgctccaggagcccccaaagaagtccctgctccagccactgagttggaagaaagtgtctcaagtcctgaagggtcagtctcggtaacccaggatggctcagctgcagagcatcagggagcccctgatgaccctattggcctctttctgatgcgaccacaggatggtgaggtgaccgtgggcggcagcattgtcttctcagcccgagtggctggggccagcctcctgaaaccgcctgtggtcaagtggttcaagggcaagtgggtggacctgagcagcaaagtgggccagcacctgcagctgcatgacagctatgacagagccagcaaggtctacttgtttgagttgcacatcacagatgctcagaccacttctgctgggggctaccgctgtgaggtgtctaccaaggacaaatttgacagctgtaacttcaacctcactgtccatgaggccattggttctggagacctggacctcagatcagctttccgacgcacgagcctggcgggagcaggtcggagaaccagtgacagccatgaagatgctgggactctggactttagttccctgctgaagaagagagacagtttccggagggactcaaagctggaggcacctgctgaagaagacgtgtgggagatcctgagacaggcaccgccgtcagaatatgagcgcatcgccttccagcacggagtcacagaccttcgaggcatgctgaagaggctcaagggcatgaagcaggatgaaaagaagagcacagcctttcagaagaagctggagcctgcctaccaggtaaacaagggccacaagattcggcttactgtggaactggctgatccggacgccgaagtcaagtggcttaagaatggacaggagatccagatgagtggcagcaagtacatcttcgagtccgtcggtgccaagcgcaccctgaccatcagccagtgctcactggctgacgacgcagcctaccagtgtgtggtggggggcgagaagtgcagcacggagctctttgtcaaagagcccccggtgctgatcactcggtccctggaagaccagctggtgatggtgggtcagcgggtggagtttgagtgtgaggtctcagaagaaggggcccaagtcaaatggctgaaggatggggttgagctgacacgtgaggagaccttcaaataccggttcaagaaagatgggcggaaacaccacttgatcatcaatgaagcaaccctggaggatgcaggacactatgcagtacgcacaagtggaggccagtcactggctgagctcattgtgcaagagaagaagttggaggtataccaaagcatcgcggacctggcagtgggagccaaggaccaggctgtgtttaagtgtgaggtttcagatgagaatgtacgcggcgtgtggctgaagaatgggaaggaactggtgcctgacaaccgcataaaggtgtcccatataggccgggtccacaaactgaccattgacgatgtcacacctgctgatgaggctgactacagctttgtccctgaagggtttgcctgcaacctgtctgccaagctccacttcatggaggtcaagattgactttgtgcctaggcaggaacctcccaagatccacttggattgtcccggcagcacaccagacaccattgtggttgttgctgggaacaagttacgcctggatgtccctatttctggagaccctgctcccactgtggtctggcagaagactgtaacacaggggaagaaggcctcaactgggccacaccctgatgccccagaagatgctggtgctgatgaggagtgggtgtttgataagaagctgttgtgtgagactgagggccgggtccgggtggagaccaccaaagaccgcagcgtctttacagtcgaaggggcagagaaggaagatgaaggtgtctacacagtcacagtaaagaaccccgtgggcgaggaccaggtcaacctcacagtcaaggtcatcgatgtcccagatgctcctgcggcccctaagatcagcaacgtgggcgaggactcctgcactgtgcagtgggaaccgcctgcctatgatggcgggcagccggtcctgggatacatcctggagcgcaagaagaaaaagagctacaggtggatgaggctcaactttgatctgctgcgggagctgagccacgaggcgaggcgcatgatcgagggtgtagcctatgagatgcgagtctacgcagtcaatgccgtgggaatgtccaggcccagccctgcctctcagcccttcatgcctattgggccccctggcgaaccaacccacttggctgtggaggatgtgtcagacaccactgtctcactcaagtggcggcccccagagcgcgtgggggccggtggcctggacggatacagcgtggagtactgccaggagggatgctccgagtggacacctgctctgcaggggctgacagagcgcacatcgatgctggtgaaggacctacccactggggcacggctgctgttccgagtacgggcacacaatgtggcaggtcctggaggccctatcgtcaccaaggagcctgtgacagtgcaggagatactgcaacgaccacggctccaactgcccagacacctgcgccagaccatccagaagaaagttggggagcctgtgaacctcctcatccctttccagggcaaaccccggcctcaggtgacctggaccaaagaggggcagcccctggcaggtgaggaggtgagcatccggaacagccccacagacacgatcttgttcatccgagctgcccgccgcacccactcgggcacctaccaggtgacagttcgcattgagaacatggaggacaaggcaacgctgatcctgcagattgtggacaagccaagtcctccccaggatatccggatcgttgagacttggggtttcaatgtggctctggagtggaagccaccccaagatgatggcaatacagagatctggggttatactgtacagaaagctgacaagaagaccatggagtggttcacggttttggaacactaccgacgcactcactgtgtggtatcagagcttatcattggcaatggctactacttccgggtcttcagccataacatggtgggttccagtgacaaagctgccgccaccaaggagccagtctttattccaagaccaggcatcacatatgagccacccaaatacaaggccctggacttctctgaggccccaagcttcacccagcccttggcaaatcgctccatcattgcaggctataatgccatcctctgctgtgctgtccgaggtagtcctaagcccaagatttcctggttcaagaatggcctggatctgggagaagatgctcgcttccgcatgttctgcaagcagggagtattgaccctggagatcaggaaaccctgcccctatgatggtggtgtctatgtctgcagggccaccaacttgcagggcgaggcacagtgtgagtgccgcctggaggtgcgagttcctcag

SEQ ID NO: 18 - DNA, 582 bp, Mus musculus
gctcctgcggcccctaagatcagcaacgtgggcgaggactcctgcactgtgcagtgggaaccgcctgcctatgatggcgggcagccggtcctgggatacatcctggagcgcaagaagaaaaagagctacaggtggatgaggctcaactttgatctgctgcgggagctgagccacgaggcgaggcgcatgatcgagggtgtagcctatgagatgcgagtctacgcagtcaatgccgtgggaatgtccaggcccagccctgcctctcagcccttcatgcctattgggccccctggcgaaccaacccacttggctgtggaggatgtgtcagacaccactgtctcactcaagtggcggcccccagagcgcgtgggggccggtggcctggacggatacagcgtggagtactgccaggagggatgctccgagtggacacctgctctgcaggggctgacagagcgcacatcgatgctggtgaaggacctacccactggggcacggctgctgttccgagtacgggcacacaatgtggcaggtcctggaggccctatcgtcaccaaggagcctgtgacagtgcaggagata

SEQ ID NO: 19 - DNA, 876 bp, Mus musculus
gctcctgcggcccctaagatcagcaacgtgggcgaggactcctgcactgtgcagtgggaaccgcctgcctatgatggcgggcagccggtcctgggatacatcctggagcgcaagaagaaaaagagctacaggtggatgaggctcaactttgatctgctgcgggagctgagccacgaggcgaggcgcatgatcgagggtgtagcctatgagatgcgagtctacgcagtcaatgccgtgggaatgtccaggcccagccctgcctctcagcccttcatgcctattgggccccctggcgaaccaacccacttggctgtggaggatgtgtcagacaccactgtctcactcaagtggcggcccccagagcgcgtgggggccggtggcctggacggatacagcgtggagtactgccaggagggatgctccgagtggacacctgctctgcaggggctgacagagcgcacatcgatgctggtgaaggacctacccactggggcacggctgctgttccgagtacgggcacacaatgtggcaggtcctggaggccctatcgtcaccaaggagcctgtgacagtgcaggagatactgcaacgaccacggctccaactgcccagacacctgcgccagaccatccagaagaaagttggggagcctgtgaacctcctcatccctttccagggcaaaccccggcctcaggtgacctggaccaaagaggggcagcccctggcaggtgaggaggtgagcatccggaacagccccacagacacgatcttgttcatccgagctgcccgccgcacccactcgggcacctaccaggtgacagttcgcattgagaacatggaggacaaggcaacgctgatcctgcagattgtggacaag

SEQ ID NO: 20 - DNA, 1170 bp, Mus musculus
gctcctgcggcccctaagatcagcaacgtgggcgaggactcctgcactgtgcagtgggaaccgcctgcctatgatggcgggcagccggtcctgggatacatcctggagcgcaagaagaaaaagagctacaggtggatgaggctcaactttgatctgctgcgggagctgagccacgaggcgaggcgcatgatcgagggtgtagcctatgagatgcgagtctacgcagtcaatgccgtgggaatgtccaggcccagccctgcctctcagcccttcatgcctattgggccccctggcgaaccaacccacttggctgtggaggatgtgtcagacaccactgtctcactcaagtggcggcccccagagcgcgtgggggccggtggcctggacggatacagcgtggagtactgccaggagggatgctccgagtggacacctgctctgcaggggctgacagagcgcacatcgatgctggtgaaggacctacccactggggcacggctgctgttccgagtacgggcacacaatgtggcaggtcctggaggccctatcgtcaccaaggagcctgtgacagtgcaggagatactgcaacgaccacggctccaactgcccagacacctgcgccagaccatccagaagaaagttggggagcctgtgaacctcctcatccctttccagggcaaaccccggcctcaggtgacctggaccaaagaggggcagcccctggcaggtgaggaggtgagcatccggaacagccccacagacacgatcttgttcatccgagctgcccgccgcacccactcgggcacctaccaggtgacagttcgcattgagaacatggaggacaaggcaacgctgatcctgcagattgtggacaagccaagtcctccccaggatatccggatcgttgagacttggggtttcaatgtggctctggagtggaagccaccccaagatgatggcaatacagagatctggggttatactgtacagaaagctgacaagaagaccatggagtggttcacggttttggaacactaccgacgcactcactgtgtggtatcagagcttatcattggcaatggctactacttccgggtcttcagccataacatggtgggttccagtgacaaagctgccgccaccaaggagccagtctttattccaagacca

SEQ ID NO: 21 - DNA, 1503 bp, Mus musculus
gctcctgcggcccctaagatcagcaacgtgggcgaggactcctgcactgtgcagtgggaaccgcctgcctatgatggcgggcagccggtcctgggatacatcctggagcgcaagaagaaaaagagctacaggtggatgaggctcaactttgatctgctgcgggagctgagccacgaggcgaggcgcatgatcgagggtgtagcctatgagatgcgagtctacgcagtcaatgccgtgggaatgtccaggcccagccctgcctctcagcccttcatgcctattgggccccctggcgaaccaacccacttggctgtggaggatgtgtcagacaccactgtctcactcaagtggcggcccccagagcgcgtgggggccggtggcctggacggatacagcgtggagtactgccaggagggatgctccgagtggacacctgctctgcaggggctgacagagcgcacatcgatgctggtgaaggacctacccactggggcacggctgctgttccgagtacgggcacacaatgtggcaggtcctggaggccctatcgtcaccaaggagcctgtgacagtgcaggagatactgcaacgaccacggctccaactgcccagacacctgcgccagaccatccagaagaaagttggggagcctgtgaacctcctcatccctttccagggcaaaccccggcctcaggtgacctggaccaaagaggggcagcccctggcaggtgaggaggtgagcatccggaacagccccacagacacgatcttgttcatccgagctgcccgccgcacccactcgggcacctaccaggtgacagttcgcattgagaacatggaggacaaggcaacgctgatcctgcagattgtggacaagccaagtcctccccaggatatccggatcgttgagacttggggtttcaatgtggctctggagtggaagccaccccaagatgatggcaatacagagatctggggttatactgtacagaaagctgacaagaagaccatggagtggttcacggttttggaacactaccgacgcactcactgtgtggtatcagagcttatcattggcaatggctactacttccgggtcttcagccataacatggtgggttccagtgacaaagctgccgccaccaaggagccagtctttattccaagaccaggcatcacatatgagccacccaaatacaaggccctggacttctctgaggccccaagcttcacccagcccttggcaaatcgctccatcattgcaggctataatgccatcctctgctgtgctgtccgaggtagtcctaagcccaagatttcctggttcaagaatggcctggatctgggagaagatgctcgcttccgcatgttctgcaagcagggagtattgaccctggagatcaggaaaccctgcccctatgatggtggtgtctatgtctgcagggccaccaacttgcagggcgaggcacagtgtgagtgccgcctggaggtgcgagttcctcag

SEQ ID NO: 22 - DNA, 912 bp, Mus musculus
ccacggctccaactgcccagacacctgcgccagaccatccagaagaaagttggggagcctgtgaacctcctcatccctttccagggcaaaccccggcctcaggtgacctggaccaaagaggggcagcccctggcaggtgaggaggtgagcatccggaacagccccacagacacgatcttgttcatccgagctgcccgccgcacccactcgggcacctaccaggtgacagttcgcattgagaacatggaggacaaggcaacgctgatcctgcagattgtggacaagccaagtcctccccaggatatccggatcgttgagacttggggtttcaatgtggctctggagtggaagccaccccaagatgatggcaatacagagatctggggttatactgtacagaaagctgacaagaagaccatggagtggttcacggttttggaacactaccgacgcactcactgtgtggtatcagagcttatcattggcaatggctactacttccgggtcttcagccataacatggtgggttccagtgacaaagctgccgccaccaaggagccagtctttattccaagaccaggcatcacatatgagccacccaaatacaaggccctggacttctctgaggccccaagcttcacccagcccttggcaaatcgctccatcattgcaggctataatgccatcctctgctgtgctgtccgaggtagtcctaagcccaagatttcctggttcaagaatggcctggatctgggagaagatgctcgcttccgcatgttctgcaagcagggagtattgaccctggagatcaggaaaccctgcccctatgatggtggtgtctatgtctgcagggccaccaacttgcagggcgaggcacagtgtgagtgccgcctggaggtgcgagttcctcag

SEQ ID NO: 23 - DNA, 621 bp, Mus musculus
cctccccaggatatccggatcgttgagacttggggtttcaatgtggctctggagtggaagccaccccaagatgatggcaatacagagatctggggttatactgtacagaaagctgacaagaagaccatggagtggttcacggttttggaacactaccgacgcactcactgtgtggtatcagagcttatcattggcaatggctactacttccgggtcttcagccataacatggtgggttccagtgacaaagctgccgccaccaaggagccagtctttattccaagaccaggcatcacatatgagccacccaaatacaaggccctggacttctctgaggccccaagcttcacccagcccttggcaaatcgctccatcattgcaggctataatgccatcctctgctgtgctgtccgaggtagtcctaagcccaagatttcctggttcaagaatggcctggatctgggagaagatgctcgcttccgcatgttctgcaagcagggagtattgaccctggagatcaggaaaccctgcccctatgatggtggtgtctatgtctgcagggccaccaacttgcagggcgaggcacagtgtgagtgccgcctggaggtgcgagttcctcag

SEQ ID NO: 24 - DNA, 282 bp, Mus musculus
ccaagcttcacccagcccttggcaaatcgctccatcattgcaggctataatgccatcctctgctgtgctgtccgaggtagtcctaagcccaagatttcctggttcaagaatggcctggatctgggagaagatgctcgcttccgcatgttctgcaagcagggagtattgaccctggagatcaggaaaccctgcccctatgatggtggtgtctatgtctgcagggccaccaacttgcagggcgaggcacagtgtgagtgccgcctggaggtgcgagttcctcag

SEQ ID NO: 25 - DNA, 3819 bp, Homo sapiens
cctgagccggggaagaagccagtctcagcttttagcaagaagccacggtcagtggaagtggccgcaggcagccctgccgtgttcgaggccgagacagagcgggcaggagtgaaggtgcgctggcagcgcggaggcagtgacatcagcgccagcaacaagtacggcctggccacagagggcacacggcatacgctgacagtgcgggaagtgggccctgccgaccagggatcttacgcagtcattgctggctcctccaaggtcaagttcgacctcaaggtcatagaggcagagaaggcagagcccatgctggcccctgcccctgcccctgctgaggccactggagcccctggagaagccccggccccagccgctgagctgggagaaagtgccccaagtcccaaagggtcaagctcagcagctctcaatggtcctacccctggagcccccgatgaccccattggcctcttcgtgatgcggccacaggatggcgaggtgaccgtgggtggcagcatcaccttctcagcccgcgtggccggcgccagcctcctgaagccgcctgtggtcaagtggttcaagggcaaatgggtggacctgagcagcaaggtgggccagcacctgcagctgcacgacagctacgaccgcgccagcaaggtctatctgttcgagctgcacatcaccgatgcccagcctgccttcactggcagctaccgctgtgaggtgtccaccaaggacaaatttgactgctccaacttcaatctcactgtccacgaggccatgggcaccggagacctggacctcctatcagccttccgccgcacgagcctggctggaggtggtcggcggatcagtgatagccatgaggacactgggattctggacttcagctcactgctgaaaaagagagacagtttccggaccccgagggactcgaagctggaggcaccagcagaggaggacgtgtgggagatcctacggcaggcacccccatctgagtacgagcgcatcgccttccagtacggcgtcactgacctgcgcggcatgctaaagaggctcaagggcatgaggcgcgatgagaagaagagcacagcctttcagaagaagctggagccggcctaccaggtgagcaaaggccacaagatccggctgaccgtggaactggctgaccatgacgctgaggtcaaatggctcaagaatggccaggagatccagatgagcggcagcaagtacatctttgagtccatcggtgccaagcgtaccctgaccatcagccagtgctcattggcggacgacgcagcctaccagtgcgtggtgggtggcgagaagtgtagcacggagctctttgtgaaagagccccctgtgctcatcacgcgccccttggaggaccagctggtgatggtggggcagcgggtggagtttgagtgtgaagtatcggaggagggggcgcaagtcaaatggctgaaggacggggtggagctgacccgggaggagaccttcaaataccggttcaagaaggacgggcagagacaccacctgatcatcaacgaggccatgctggaggacgcggggcactatgcactgtgcactagcgggggccaggcgctggctgagctcattgtgcaggaaaagaagctggaggtgtaccagagcatcgcagacctgatggtgggcgcaaaggaccaggcggtgttcaaatgtgaggtctcagatgagaatgttcggggtgtgtggctgaagaatgggaaggagctggtgcccgacagccgcataaaggtgtcccacatcgggcgggtccacaaactgaccattgacgacgtcacacctgccgacgaggctgactacagctttgtgcccgagggcttcgcctgcaacctgtcagccaagctccacttcatggaggtcaagattgacttcgtacccaggcaggaacctcccaagatccacctggactgcccaggccgcataccagacaccattgtggttgtagctggaaataagctacgtctggacgtccctatctctggggaccctgctcccactgtgatctggcagaaggctatcacgcaggggaataaggccccagccaggccagccccagatgccccagaggacacaggtgacagcgatgagtgggtgtttgacaagaagctgctgtgtgagaccgagggccgggtccgcgtggagaccaccaaggaccgcagcatcttcacggtcgagggggcagagaaggaagatgagggcgtctacacggtcacagtgaagaaccctgtgggcgaggaccaggtcaacctcacagtcaaggtcatcgacgtgccagacgcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcag

SEQ ID NO: 26 - DNA, 582 bp, Homo sapiens
gcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatc

SEQ ID NO: 27 - DNA, 876 bp, Homo sapiens
gcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaag

SEQ ID NO: 28 - DNA, 1170 bp, Homo sapiens
gcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagacca

SEQ ID NO: 29 - DNA, 1503 bp, Homo sapiens
gcacctgcggcccccaagatcagcaacgtgggagaggactcctgcacagtacagtgggagccgcctgcctacgatggcgggcagcccatcctgggctacatcctggagcgcaagaagaagaagagctaccggtggatgcggctgaacttcgacctgattcaggagctgagtcatgaagcgcggcgcatgatcgagggcgtggtgtacgagatgcgcgtctacgcggtcaacgccatcggcatgtccaggcccagccctgcctcccagcccttcatgcctatcggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcag

SEQ ID NO: 30 - DNA, 912 bp, Homo sapiens
ccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcag

SEQ ID NO: 31 - DNA, 621 bp, Homo sapiens
cctccccaggatctccgggtgactgacgcctggggtcttaatgtggctctggagtggaagccaccccaggatgtcggcaacacggagctctgggggtacacagtgcagaaagccgacaagaagaccatggagtggttcaccgtcttggagcattaccgccgcacccactgcgtggtgccagagctcatcattggcaatggctactacttccgcgtcttcagccagaatatggttggctttagtgacagagcggccaccaccaaggagcccgtctttatccccagaccaggcatcacctatgagccacccaactataaggccctggacttctccgaggccccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcag

SEQ ID NO: 32 - DNA, 282 bp, Homo sapiens
ccaagcttcacccagcccctggtgaaccgctcggtcatcgcgggctacactgctatgctctgctgtgctgtccggggtagccccaagcccaagatttcctggttcaagaatggcctggacctgggagaagacgcccgcttccgcatgttcagcaagcagggagtgttgactctggagattagaaagccctgcccctttgacgggggcatctatgtctgcagggccaccaacttacagggcgaggcacggtgtgagtgccgcctggaggtgcgagtgcctcag

SEQ ID NO: 33 - DNA, 168 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacg

SEQ ID NO: 34 - DNA, 106 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgg

SEQ ID NO: 35 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 36 - protein, 735 aa, Artificial Sequence [sequence omitted]

SEQ ID NO: 37 - protein, 736 aa, Artificial Sequence
  Synthetic
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 38 - protein, 734 aa, Artificial Sequence
  Synthetic
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 39 - protein, 724 aa, Artificial Sequence
  Synthetic
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 40 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 41 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 42 - protein, 737 aa, Artificial Sequence
  Synthetic
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 43 - protein, 738 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 44 - protein, 736 aa, Artificial Sequence
  Synthetic
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 46 - protein, 738 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 47 - protein, 738 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 48 - protein, 737 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLEAGDNPYLRYNHADAEFQERLQEDTSFGGNLGRAVFQAKKRVLEPLGLVEEGAKTAPGKKRPVEQSPQEPDSSSGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 49 - protein, 735 aa, Artificial Sequence
  Synthetic
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATTDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 50 - protein, 735 aa, Artificial Sequence
  Synthetic
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNAPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATTDVNTQGVLPGMVWQDRDVYLQGPIWAKIPHTDGHFHPSPLMGGFGLKHPPPQILIKNTPVPANPSTTFSAAKLASFITQYSTGQVSVEIEWELQKENSKRWNPEIQYTSNYNKSVNVDFTVDTNGVYSEPRPIGTRYLTRNL

SEQ ID NO: 51 - protein, 735 aa, Artificial Sequence
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLARAVFQAKKRVLEPLGLVEEPVKTAPGKKRPVEHSPAEPDSSSGTGKSGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQTVGRSSFYCLEYFPSQMLRTGNNFTFSYTFEDVPFHSSYAHSQSLDRLMNPLIDQYLYYLSRTNTPSGTTTQSRLRFSQAGASDIRDQSRNWLPGPCYRQQRVSKTAADNNNSDYSWTGATKYHLNGRDSLVNPGTAMASHKDDEEKYFPQSGVLIFGKQDSGKTNVDIERVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 52 - protein, 735 aa, Artificial Sequence
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQQQNTAPATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 53 - protein, 200 aa, Homo sapiens
PPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSP

SEQ ID NO: 54 - protein, 104 aa, Homo sapiens
PPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPR

SEQ ID NO: 55 - protein, 102 aa, Homo sapiens
ILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSP

SEQ ID NO: 56 - protein, 406 aa, Homo sapiens
PPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 57 - protein, 404 aa, Homo sapiens
VPDAPAAPKISNVGEDSCTVQWEPPAYDGGQPILGYILERKKKKSYRWMRLNFDLIQELSHEARRMIEGVVYEMRVYAVNAIGMSRPSPASQPFMPILQRPRLQLPRHLRQTIQKKVGEPVNLLIPFQGKPRPQVTWTKEGQPLAGEEVSIRNSPTDTILFIRAARRVHSGTYQVTVRIENMEDKATLVLQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 58 - protein, 416 aa, Homo sapiens
VPDAPAAPKISNVGEDSCTVQWEPPAYDGGQPILGYILERKKKKSYRWMRLNFDLIQELSHEARRMIEGVVYEMRVYAVNAIGMSRPSPASQPFMPIGPPSEPTHLAVEDVSDTTVSLKWRPPERVGAGGLDGYSVEYCPEGCSEWVAALQGLTEHTSILVKDLPTGARLLFRVRAHNMAGPGAPVTTTEPVTVQEILQRPRQVVDKPSPPQDLRVTDAWGLNVALEWKPPQDVGNTELWGYTVQKADKKTMEWFTVLEHYRRTHCVVPELIIGNGYYFRVFSQNMVGFSDRAATTKEPVFIPRPGITYEPPNYKALDFSEAPSFTQPLVNRSVIAGYTAMLCCAVRGSPKPKISWFKNGLDLGEDARFRMFSKQGVLTLEIRKPCPFDGGIYVCRATNLQGEARCECRLEVRVPQ

SEQ ID NO: 59 - protein, 200 aa, Mus musculus
PPGEPTHLAVEDVSDTTVSLKWRPPERVGAGGLDGYSVEYCQEGCSEWTPALQGLTERTSMLVKDLPTGARLLFRVRAHNVAGPGGPIVTKEPVTVQEILQRPRLQLPRHLRQTIQKKVGEPVNLLIPFQGKPRPQVTWTKEGQPLAGEEVSIRNSPTDTILFIRAARRTHSGTYQVTVRIENMEDKATLILQIVDKPSP

SEQ ID NO: 60 - protein, 104 aa, Mus musculus
PPGEPTHLAVEDVSDTTVSLKWRPPERVGAGGLDGYSVEYCQEGCSEWTPALQGLTERTSMLVKDLPTGARLLFRVRAHNVAGPGGPIVTKEPVTVQEILQRPR

SEQ ID NO: 61 - protein, 102 aa, Mus musculus
ILQRPRLQLPRHLRQTIQKKVGEPVNLLIPFQGKPRPQVTWTKEGQPLAGEEVSIRNSPTDTILFIRAARRTHSGTYQVTVRIENMEDKATLILQIVDKPSP

SEQ ID NO: 62 - protein, 406 aa, Mus musculus
PPGEPTHLAVEDVSDTTVSLKWRPPERVGAGGLDGYSVEYCQEGCSEWTPALQGLTERTSMLVKDLPTGARLLFRVRAHNVAGPGGPIVTKEPVTVQEILQRPRLQLPRHLRQTIQKKVGEPVNLLIPFQGKPRPQVTWTKEGQPLAGEEVSIRNSPTDTILFIRAARRTHSGTYQVTVRIENMEDKATLILQIVDKPSPPQDIRIVETWGFNVALEWKPPQDDGNTEIWGYTVQKADKKTMEWFTVLEHYRRTHCVVSELIIGNGYYFRVFSHNMVGSSDKAAATKEPVFIPRPGITYEPPKYKALDFSEAPSFTQPLANRSIIAGYNAILCCAVRGSPKPKISWFKNGLDLGEDARFRMFCKQGVLTLEIRKPCPYDGGVYVCRATNLQGEAQCECRLEVRVPQ

SEQ ID NO: 63 - protein, 404 aa, Mus musculus
VPDAPAAPKISNVGEDSCTVQWEPPAYDGGQPVLGYILERKKKKSYRWMRLNFDLLRELSHEARRMIEGVAYEMRVYAVNAVGMSRPSPASQPFMPILQRPRLQLPRHLRQTIQKKVGEPVNLLIPFQGKPRPQVTWTKEGQPLAGEEVSIRNSPTDTILFIRAARRTHSGTYQVTVRIENMEDKATLILQIVDKPSPPQDIRIVETWGFNVALEWKPPQDDGNTEIWGYTVQKADKKTMEWFTVLEHYRRTHCVVSELIIGNGYYFRVFSHNMVGSSDKAAATKEPVFIPRPGITYEPPKYKALDFSEAPSFTQPLANRSIIAGYNAILCCAVRGSPKPKISWFKNGLDLGEDARFRMFCKQGVLTLEIRKPCPYDGGVYVCRATNLQGEAQCECRLEVRVPQ

SEQ ID NO: 64 - protein, 416 aa, Mus musculus
VPDAPAAPKISNVGEDSCTVQWEPPAYDGGQPVLGYILERKKKKSYRWMRLNFDLLRELSHEARRMIEGVAYEMRVYAVNAVGMSRPSPASQPFMPIGPPGEPTHLAVEDVSDTTVSLKWRPPERVGAGGLDGYSVEYCQEGCSEWTPALQGLTERTSMLVKDLPTGARLLFRVRAHNVAGPGGPIVTKEPVTVQEILQRPRQIVDKPSPPQDIRIVETWGFNVALEWKPPQDDGNTEIWGYTVQKADKKTMEWFTVLEHYRRTHCVVSELIIGNGYYFRVFSHNMVGSSDKAAATKEPVFIPRPGITYEPPKYKALDFSEAPSFTQPLANRSIIAGYNAILCCAVRGSPKPKISWFKNGLDLGEDARFRMFCKQGVLTLEIRKPCPYDGGVYVCRATNLQGEAQCECRLEVRVPQ

SEQ ID NO: 65 - RNA, 600 nt, Homo sapiens
ccccccagcgaacccacccaccuggcaguagaggacgucucugacaccacggucucccucaaguggcggcccccagagcgcgugggagcaggaggccuggauggcuacagcguggaguacugcccagagggcugcucagaguggguggcugcccugcaggggcugacagagcacacaucgauacuggugaaggaccugcccacgggggcccggcugcuuuuccgagugcgggcacacaauauggcagggccuggagccccuguuaccaccacggagccggugacagugcaggagauccugcaacggccacggcuucagcugcccaggcaccugcgccagaccauucagaagaaggucggggagccugugaaccuucucaucccuuuccagggcaagccccggccucaggugaccuggaccaaagaggggcagccccuggcaggcgaggaggugagcauccgcaacagccccacagacaccauccuguucauccgggccgcucgccgcgugcauucaggcacuuaccaggugacggugcgcauugagaacauggaggacaaggccacgcuggugcugcagguuguugacaagccaaguccu

SEQ ID NO: 66 - RNA, 312 nt, Homo sapiens
ccccccagcgaacccacccaccuggcaguagaggacgucucugacaccacggucucccucaaguggcggcccccagagcgcgugggagcaggaggccuggauggcuacagcguggaguacugcccagagggcugcucagaguggguggcugcccugcaggggcugacagagcacacaucgauacuggugaaggaccugcccacgggggcccggcugcuuuuccgagugcgggcacacaauauggcagggccuggagccccuguuaccaccacggagccggugacagugcaggagauccugcaacggccacgg

SEQ ID NO: 67 - RNA, 306 nt, Homo sapiens
auccugcaacggccacggcuucagcugcccaggcaccugcgccagaccauucagaagaaggucggggagccugugaaccuucucaucccuuuccagggcaagccccggccucaggugaccuggaccaaagaggggcagccccuggcaggcgaggaggugagcauccgcaacagccccacagacaccauccuguucauccgggccgcucgccgcgugcauucaggcacuuaccaggugacggugcgcauugagaacauggaggacaaggccacgcuggugcugcagguuguugacaagccaaguccu

SEQ ID NO: 68 - RNA, 1218 nt, Homo sapiens
ccccccagcgaacccacccaccuggcaguagaggacgucucugacaccacggucucccucaaguggcggcccccagagcgcgugggagcaggaggccuggauggcuacagcguggaguacugcccagagggcugcucagaguggguggcugcccugcaggggcugacagagcacacaucgauacuggugaaggaccugcccacgggggcccggcugcuuuuccgagugcgggcacacaauauggcagggccuggagccccuguuaccaccacggagccggugacagugcaggagauccugcaacggccacggcuucagcugcccaggcaccugcgccagaccauucagaagaaggucggggagccugugaaccuucucaucccuuuccagggcaagccccggccucaggugaccuggaccaaagaggggcagccccuggcaggcgaggaggugagcauccgcaacagccccacagacaccauccuguucauccgggccgcucgccgcgugcauucaggcacuuaccaggugacggugcgcauugagaacauggaggacaaggccacgcuggugcugcagguuguugacaagccaaguccuccccaggaucuccgggugacugacgccuggggucuuaauguggcucuggaguggaagccaccccaggaugucggcaacacggagcucuggggguacacagugcagaaagccgacaagaagaccauggagugguucaccgucuuggagcauuaccgccgcacccacugcguggugccagagcucaucauuggcaauggcuacuacuuccgcgucuucagccagaauaugguuggcuuuagugacagagcggccaccaccaaggagcccgucuuuauccccagaccaggcaucaccuaugagccacccaacuauaaggcccuggacuucuccgaggccccaagcuucacccagccccuggugaaccgcucggucaucgcgggcuacacugcuaugcucugcugugcuguccgggguagccccaagcccaagauuuccugguucaagaauggccuggaccugggagaagacgcccgcuuccgcauguucagcaagcagggaguguugacucuggagauuagaaagcccugccccuuugacgggggcaucuaugucugcagggccaccaacuuacagggcgaggcacggugugagugccgccuggaggugcgagugccucag

SEQ ID NO: 69 - RNA, 1215 nt, Homo sapiens
gugccagacgcaccugcggcccccaagaucagcaacgugggagaggacuccugcacaguacagugggagccgccugccuacgauggcgggcagcccauccugggcuacauccuggagcgcaagaagaagaagagcuaccgguggaugcggcugaacuucgaccugauucaggagcugagucaugaagcgcggcgcaugaucgagggcgugguguacgagaugcgcgucuacgcggucaacgccaucggcauguccaggcccagcccugccucccagcccuucaugccuauccugcaacggccacggcuucagcugcccaggcaccugcgccagaccauucagaagaaggucggggagccugugaaccuucucaucccuuuccagggcaagccccggccucaggugaccuggaccaaagaggggcagccccuggcaggcgaggaggugagcauccgcaacagccccacagacaccauccuguucauccgggccgcucgccgcgugcauucaggcacuuaccaggugacggugcgcauugagaacauggaggacaaggccacgcuggugcugcagguuguugacaagccaaguccuccccaggaucuccgggugacugacgccuggggucuuaauguggcucuggaguggaagccaccccaggaugucggcaacacggagcucuggggguacacagugcagaaagccgacaagaagaccauggagugguucaccgucuuggagcauuaccgccgcacccacugcguggugccagagcucaucauuggcaauggcuacuacuuccgcgucuucagccagaauaugguuggcuuuagugacagagcggccaccaccaaggagcccgucuuuauccccagaccaggcaucaccuaugagccacccaacuauaaggcccuggacuucuccgaggccccaagcuucacccagccccuggugaaccgcucggucaucgcgggcuacacugcuaugcucugcugugcuguccgggguagccccaagcccaagauuuccugguucaagaauggccuggaccugggagaagacgcccgcuuccgcauguucagcaagcagggaguguugacucuggagauuagaaagcccugccccuuugacgggggcaucuaugucugcagggccaccaacuuacagggcgaggcacggugugagugccgccuggaggugcgagugccucaguga

SEQ ID NO: 70 - RNA, 1248 nt, Homo sapiens
gugccagacgcaccugcggcccccaagaucagcaacgugggagaggacuccugcacaguacagugggagccgccugccuacgauggcgggcagcccauccugggcuacauccuggagcgcaagaagaagaagagcuaccgguggaugcggcugaacuucgaccugauucaggagcugagucaugaagcgcggcgcaugaucgagggcgugguguacgagaugcgcgucuacgcggucaacgccaucggcauguccaggcccagcccugccucccagcccuucaugccuaucgguccccccagcgaacccacccaccuggcaguagaggacgucucugacaccacggucucccucaaguggcggcccccagagcgcgugggagcaggaggccuggauggcuacagcguggaguacugcccagagggcugcucagaguggguggcugcccugcaggggcugacagagcacacaucgauacuggugaaggaccugcccacgggggcccggcugcuuuuccgagugcgggcacacaauauggcagggccuggagccccuguuaccaccacggagccggugacagugcaggagauccugcaacggccacggcagguuguugacaagccaaguccuccccaggaucuccgggugacugacgccuggggucuuaauguggcucuggaguggaagccaccccaggaugucggcaacacggagcucuggggguacacagugcagaaagccgacaagaagaccauggagugguucaccgucuuggagcauuaccgccgcacccacugcguggugccagagcucaucauuggcaauggcuacuacuuccgcgucuucagccagaauaugguuggcuuuagugacagagcggccaccaccaaggagcccgucuuuauccccagaccaggcaucaccuaugagccacccaacuauaaggcccuggacuucuccgaggccccaagcuucacccagccccuggugaaccgcucggucaucgcgggcuacacugcuaugcucugcugugcuguccgggguagccccaagcccaagauuuccugguucaagaauggccuggaccugggagaagacgcccgcuuccgcauguucagcaagcagggaguguugacucuggagauuagaaagcccugccccuuugacgggggcaucuaugucugcagggccaccaacuuacagggcgaggcacggugugagugccgccuggaggugcgagugccucag

SEQ ID NO: 71 - RNA, 600 nt, Mus musculus
cccccuggcgaaccaacccacuuggcuguggaggaugugucagacaccacugucucacucaaguggcggcccccagagcgcgugggggccgguggccuggacggauacagcguggaguacugccaggagggaugcuccgaguggacaccugcucugcaggggcugacagagcgcacaucgaugcuggugaaggaccuacccacuggggcacggcugcuguuccgaguacgggcacacaauguggcagguccuggaggcccuaucgucaccaaggagccugugacagugcaggagauacugcaacgaccacggcuccaacugcccagacaccugcgccagaccauccagaagaaaguuggggagccugugaaccuccucaucccuuuccagggcaaaccccggccucaggugaccuggaccaaagaggggcagccccuggcaggugaggaggugagcauccggaacagccccacagacacgaucuuguucauccgagcugcccgccgcacccacucgggcaccuaccaggugacaguucgcauugagaacauggaggacaaggcaacgcugauccugcagauuguggacaagccaaguccu

SEQ ID NO: 72 - RNA, 312 nt, Mus musculus
cccccuggcgaaccaacccacuuggcuguggaggaugugucagacaccacugucucacucaaguggcggcccccagagcgcgugggggccgguggccuggacggauacagcguggaguacugccaggagggaugcuccgaguggacaccugcucugcaggggcugacagagcgcacaucgaugcuggugaaggaccuacccacuggggcacggcugcuguuccgaguacgggcacacaauguggcagguccuggaggcccuaucgucaccaaggagccugugacagugcaggagauacugcaacgaccacgg

SEQ ID NO: 73 - RNA, 306 nt, Mus musculus
auacugcaacgaccacggcuccaacugcccagacaccugcgccagaccauccagaagaaaguuggggagccugugaaccuccucaucccuuuccagggcaaaccccggccucaggugaccuggaccaaagaggggcagccccuggcaggugaggaggugagcauccggaacagccccacagacacgaucuuguucauccgagcugcccgccgcacccacucgggcaccuaccaggugacaguucgcauugagaacauggaggacaaggcaacgcugauccugcagauuguggacaagccaaguccu

SEQ ID NO: 74 - RNA, 1218 nt, Mus musculus
cccccuggcgaaccaacccacuuggcuguggaggaugugucagacaccacugucucacucaaguggcggcccccagagcgcgugggggccgguggccuggacggauacagcguggaguacugccaggagggaugcuccgaguggacaccugcucugcaggggcugacagagcgcacaucgaugcuggugaaggaccuacccacuggggcacggcugcuguuccgaguacgggcacacaauguggcagguccuggaggcccuaucgucaccaaggagccugugacagugcaggagauacugcaacgaccacggcuccaacugcccagacaccugcgccagaccauccagaagaaaguuggggagccugugaaccuccucaucccuuuccagggcaaaccccggccucaggugaccuggaccaaagaggggcagccccuggcaggugaggaggugagcauccggaacagccccacagacacgaucuuguucauccgagcugcccgccgcacccacucgggcaccuaccaggugacaguucgcauugagaacauggaggacaaggcaacgcugauccugcagauuguggacaagccaaguccuccccaggauauccggaucguugagacuugggguuucaauguggcucuggaguggaagccaccccaagaugauggcaauacagagaucugggguuauacuguacagaaagcugacaagaagaccauggagugguucacgguuuuggaacacuaccgacgcacucacugugugguaucagagcuuaucauuggcaauggcuacuacuuccgggucuucagccauaacaugguggguuccagugacaaagcugccgccaccaaggagccagucuuuauuccaagaccaggcaucacauaugagccacccaaauacaaggcccuggacuucucugaggccccaagcuucacccagcccuuggcaaaucgcuccaucauugcaggcuauaaugccauccucugcugugcuguccgagguaguccuaagcccaagauuuccugguucaagaauggccuggaucugggagaagaugcucgcuuccgcauguucugcaagcagggaguauugacccuggagaucaggaaacccugccccuaugaugguggugucuaugucugcagggccaccaacuugcagggcgaggcacagugugagugccgccuggaggugcgaguuccucag

SEQ ID NO: 75 - RNA, 1212 nt, Mus musculus
gucccagaugcuccugcggccccuaagaucagcaacgugggcgaggacuccugcacugugcagugggaaccgccugccuaugauggcgggcagccgguccugggauacauccuggagcgcaagaagaaaaagagcuacagguggaugaggcucaacuuugaucugcugcgggagcugagccacgaggcgaggcgcaugaucgaggguguagccuaugagaugcgagucuacgcagucaaugccgugggaauguccaggcccagcccugccucucagcccuucaugccuauacugcaacgaccacggcuccaacugcccagacaccugcgccagaccauccagaagaaaguuggggagccugugaaccuccucaucccuuuccagggcaaaccccggccucaggugaccuggaccaaagaggggcagccccuggcaggugaggaggugagcauccggaacagccccacagacacgaucuuguucauccgagcugcccgccgcacccacucgggcaccuaccaggugacaguucgcauugagaacauggaggacaaggcaacgcugauccugcagauuguggacaagccaaguccuccccaggauauccggaucguugagacuugggguuucaauguggcucuggaguggaagccaccccaagaugauggcaauacagagaucugggguuauacuguacagaaagcugacaagaagaccauggagugguucacgguuuuggaacacuaccgacgcacucacugugugguaucagagcuuaucauuggcaauggcuacuacuuccgggucuucagccauaacaugguggguuccagugacaaagcugccgccaccaaggagccagucuuuauuccaagaccaggcaucacauaugagccacccaaauacaaggcccuggacuucucugaggccccaagcuucacccagcccuuggcaaaucgcuccaucauugcaggcuauaaugccauccucugcugugcuguccgagguaguccuaagcccaagauuuccugguucaagaauggccuggaucugggagaagaugcucgcuuccgcauguucugcaagcagggaguauugacccuggagaucaggaaacccugccccuaugaugguggugucuaugucugcagggccaccaacuugcagggcgaggcacagugugagugccgccuggaggugcgaguuccucag

SEQ ID NO: 76 - RNA, 1248 nt, Mus musculus
gucccagaugcuccugcggccccuaagaucagcaacgugggcgaggacuccugcacugugcagugggaaccgccugccuaugauggcgggcagccgguccugggauacauccuggagcgcaagaagaaaaagagcuacagguggaugaggcucaacuuugaucugcugcgggagcugagccacgaggcgaggcgcaugaucgaggguguagccuaugagaugcgagucuacgcagucaaugccgugggaauguccaggcccagcccugccucucagcccuucaugccuauugggcccccuggcgaaccaacccacuuggcuguggaggaugugucagacaccacugucucacucaaguggcggcccccagagcgcgugggggccgguggccuggacggauacagcguggaguacugccaggagggaugcuccgaguggacaccugcucugcaggggcugacagagcgcacaucgaugcuggugaaggaccuacccacuggggcacggcugcuguuccgaguacgggcacacaauguggcagguccuggaggcccuaucgucaccaaggagccugugacagugcaggagauacugcaacgaccacggcagauuguggacaagccaaguccuccccaggauauccggaucguugagacuugggguuucaauguggcucuggaguggaagccaccccaagaugauggcaauacagagaucugggguuauacuguacagaaagcugacaagaagaccauggagugguucacgguuuuggaacacuaccgacgcacucacugugugguaucagagcuuaucauuggcaauggcuacuacuuccgggucuucagccauaacaugguggguuccagugacaaagcugccgccaccaaggagccagucuuuauuccaagaccaggcaucacauaugagccacccaaauacaaggcccuggacuucucugaggccccaagcuucacccagcccuuggcaaaucgcuccaucauugcaggcuauaaugccauccucugcugugcuguccgagguaguccuaagcccaagauuuccugguucaagaauggccuggaucugggagaagaugcucgcuuccgcauguucugcaagcagggaguauugacccuggagaucaggaaacccugccccuaugaugguggugucuaugucugcagggccaccaacuugcagggcgaggcacagugugagugccgccuggaggugcgaguuccucag

SEQ ID NO: 77 - DNA, 5767 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttacgggccccccctcgaggtcgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaagcggccgcactactccgcggactactactagtatggccgtttacccatacgatgttcctgactatgcgggctatccctatgacgtcccggactatgcaggatcctatccatatgacgttccagattacgctaccggtccccccagcgaacccacccacctggcagtagaggacgtctctgacaccacggtctccctcaagtggcggcccccagagcgcgtgggagcaggaggcctggatggctacagcgtggagtactgcccagagggctgctcagagtgggtggctgccctgcaggggctgacagagcacacatcgatactggtgaaggacctgcccacgggggcccggctgcttttccgagtgcgggcacacaatatggcagggcctggagcccctgttaccaccacggagccggtgacagtgcaggagatcctgcaacggccacggcttcagctgcccaggcacctgcgccagaccattcagaagaaggtcggggagcctgtgaaccttctcatccctttccagggcaagccccggcctcaggtgacctggaccaaagaggggcagcccctggcaggcgaggaggtgagcatccgcaacagccccacagacaccatcctgttcatccgggccgctcgccgcgtgcattcaggcacttaccaggtgacggtgcgcattgagaacatggaggacaaggccacgctggtgctgcaggttgttgacaagccaagtcctaagcttggacaattgggagagctcggatccggagccacgaacttctctctgttaaagcaagcaggagacgtggaagaaaaccccggtcctgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagctcgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 78 - DNA, 5749 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttacgggccccccctcgaggtcgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgatccccgaagctctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaagcggccgcactactccgcggactactactagtatggccgtttacccatacgatgttcctgactatgcgggctatccctatgacgtcccggactatgcaggatcctatccatatgacgttccagattacgctaccggtttcatgcctattgggccccctggcgaaccaacccacttggctgtggaggatgtgtcagacaccactgtctcactcaagtggcggcccccagagcgcgtgggggccggtggcctggacggatacagcgtggagtactgccaggagggatgctccgagtggacacctgctctgcaggggctgacagagcgcacatcgatgctggtgaaggacctacccactggggcacggctgctgttccgagtacgggcacacaatgtggcaggtcctggaggccctatcgtcaccaaggagcctgtgacagtgcaggagatactgcaacgaccacggctccaactgcccagacacctgcgccagaccatccagaagaaagttggggagcctgtgaacctcctcatccctttccagggcaaaccccggcctcaggtgacctggaccaaagaggggcagcccctggcaggtgaggaggtgagcatccggaacagccccacagacacgatcttgttcatccgagctgcccgccgcacccactcgggcacctaccaggtgacagttcgcattgagaacatggaggacaaggcaacgaagcttggacaattgggagagctcggatccggagccacgaacttctctctgttaaagcaagcaggagacgtggaagaaaaccccggtcctgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagctcgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

(19) United States
(12) Patent Application Publication
(10) Pub. No.: US 2022/0023384 A1
(43) Pub. Date: Jan. 27, 2022

(54) MYBPC3 POLYPEPTIDES AND USES THEREOF

(71) Applicant: Children's Medical Center Corporation, Boston, MA (US)

(72) Inventors: William T. Pu, Chestnut Hill, MA (US); Fujian Lu, Brookline, MA (US); Vassilios Bezzerides, Brookline, MA (US)

(73) Assignee: Children's Medical Center Corporation, Boston, MA (US)

(21) Appl. No.: 17/371,017

(22) Filed: Jul. 8, 2021

Related U.S. Application Data
(60) Provisional application No. 63/049,398, filed on Jul. 8, 2020.

(51) Int. Cl. A61K 38/17; C12N 15/86; A61P 9/06

(52) U.S. Cl. CPC A61K 38/1719; C12N 15/86; A61K 48/00; C12N 2750/14143; A61P 9/06

(57) ABSTRACT

Provided herein are compositions and methods for treating a disorder associated with abnormal RYR2 function (e.g., arrhythmia or heart failure). In some embodiments, method comprises administering to a subject in need thereof an effective amount of a polypeptide comprising a C-terminal domain of Cardiac Myosin binding protein C (MYBPC3) or a nucleic acid or an rAAV encoding such polypeptide.

### RELATED APPLICATIONS

This application claims the benefit under 35 U.S.C. § 119(e) of U.S. provisional application No. 63/049,398, filed Jul. 8, 2020, which is incorporated by reference herein in its entirety.

### GOVERNMENT SUPPORT

This invention was made with government support under Grant Nos. R01HL146634 and UG3HL141798 awarded by the National Institutes of Health. The Government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jul. 6, 2021, is named C123370191WO00-SEQ-RE and is 257,367 bytes in size.

### BACKGROUND

Many forms of heart disease and heart arrhythmia are caused directly or indirectly by improper regulation of Ca²⁺ release in heart muscle cells. Ca²⁺ release in heart muscle cells occurs in specialized structures known as dyads. A key regulator of Ca²⁺ release is RYR2 (ryanodine receptor type 2), a Ca²⁺ channel through which Ca²⁺ is released from the sarcoplasmic reticulum into the cytoplasm. One example of heart arrhythmia caused abnormal Ca²⁺ release is CPVT (Catecholaminergic Polymorphic Ventricular Tachycardia), a malignant inherited arrhythmia in which patients are at risk for lethal arrhythmias during exercise. CPVT has an estimated prevalence of 1:10000 and causes about 15% of autopsy negative cases of sudden unexplained death in the young. 60% of CPVT cases are caused by mutations in RYR2. Within RYR2, over 160 different mutations, clustered within 4 “hotspot” regions of the coding sequence, cause CPVT. Currently CPVT is not adequately treated by available options, and patients continue to suffer from sudden death or aborted sudden death, as well as morbidities arising from current therapies. Other forms of arrhythmia, such as atrial fibrillation, involve abnormal regulation of Ca2+ release from RYR2. Abnormal Ca²⁺ release from RYR2 can also contribute to contractile dysfunction in inherited and acquired forms of heart failure.

### SUMMARY

The present disclosure is based, at least in part, on the surprising finding of an interaction between the C-terminus of an endogenous cardiac protein MYBPC3 and RYR2, and that overexpression of this interacting domain suppressed aberrant RYR2 activity and alleviated arrhythmia. In some aspects, the present disclosure provides compositions and methods for treating a disorder associated with abnormal RYR2 function (e.g., arrhythmia or heart failure that are either inherited or acquired). In some embodiments, the subject treated using the methods described herein is a subject with arrhythmia whose response to existing medical management is sub-optimal.

Some aspects of the present disclosure provide methods of treating a disorder associated with abnormal ryanodine receptor type 2 (RYR2) function. In some embodiments, the method comprises administering to a subject in need thereof an effective amount of a polypeptide comprising a C-terminal domain of Cardiac Myosin binding protein C (MYBPC3). In some embodiments, the method comprises administering to a subject in need thereof an effective amount of a nucleic acid comprising a nucleotide sequence encoding a polypeptide comprising a C-terminal domain of Cardiac Myosin binding protein C (MYBPC3).

In some embodiments, the abnormal RYR2 function is caused by one or more mutations in RYR2. In some embodiments, the mutation in RYR2 causes excessive diastolic Ca²⁺ release in cardiomyocytes in the subject.

In some embodiments, the polypeptide comprises an amino acid sequence that is at least 80% identical to any one of SEQ ID NOs: 1-16 or 53-64. In some embodiments, the polypeptide comprises the amino acid sequence of any one of SEQ ID NOs: 1-16 or 53-64.

In some embodiments, the nucleotide sequence is operably linked to a promoter. In some embodiments, the nucleic acid is a vector. In some embodiments, the vector is an expression vector. In some embodiments, the expression vector is a viral vector. In some embodiments, the viral vector is selected from a lentiviral vector, a retroviral vector, or a recombinant adeno-associated virus (rAAV) vector.

In some embodiments, the viral vector is a rAAV vector further comprising two AAV inverted terminal repeats (ITRs) flanking the nucleotide sequence encoding the polypeptide and the promoter. In some embodiments, wherein the rAAV vector is packaged in a rAAV particle. In some embodiments, the rAAV particle further comprises a capsid protein. In some embodiments, the capsid protein is of a serotype selected from AAV1, AAV2, AAV3, AAV4, AAV5, AAV6, AAV6.2, AAV7, AAV8, AAV9, AAV.rh8, AAV.rh10, AAV.rh39, AAV.43, AAV2/2-66, AAV2/2-84, and AAV2/2-125, or a variant thereof. In some embodiments, the capsid protein is of a serotype AAV9. In some embodiments, the rAAV is a self-complementary AAV (scAAV). In some embodiments, the nucleotide sequence encoding the polypeptide is codon-optimized. In some embodiments, the nucleic acid is a messenger RNA (mRNA). In some embodiments, the mRNA is a modified mRNA.

In some embodiments, the polypeptide or the nucleic acid is delivered to a cardiomyocyte in the subject.

In some embodiments, the disorder is arrhythmia. In some embodiments, the arrhythmia is inherited or acquired. In some embodiments, the inherited arrhythmia is Catecholaminergic Polymorphic Ventricular Tachycardia (CPVT). In some embodiments, the acquired arrhythmia is a ventricular arrhythmia or a supraventricular arrhythmia. In some embodiments, the ventricular arrhythmia is ventricular tachycardia, ventricular fibrillation, or premature ventricular contraction. In some embodiments, the supraventricular arrhythmia is atrial fibrillation, atrial flutter, atrial tachycardia, premature atrial contraction, or paroxysmal supraventricular tachycardia. In some embodiments, the disorder is heart failure.

In some embodiments, administering the polypeptide or the nucleic acid reduces the excessive diastolic Ca²⁺ release in cardiomyocytes in the subject.

In some embodiments, the subject is human. In some embodiments, the administering is via injection.

Some aspects of the present disclosure provide methods of treating arrhythmia, the method comprises administering to a subject in need thereof an effective amount of a recombinant adeno-associated virus (rAAV), wherein the rAAV comprises a capsid protein of serotype AAV9 and a nucleotide sequence encoding a polypeptide comprising a C-terminal domain of Cardiac Myosin binding protein C (MYBPC3).

Other aspects of the present disclosure provide recombinant adeno-associated virus (rAAV) comprising a capsid protein and a nucleotide sequence encoding a polypeptide comprising a C-terminal domain of Cardiac Myosin binding protein C (MYBPC3).

In some embodiments, the polypeptide comprises the amino acid sequence of any one of SEQ ID NOs: 1-16 or 53-64.

Further provided herein are uses of the rAAV described herein in treating a disorder associated with abnormal ryanodine receptor type 2 (RYR2) function. In some embodiments, the disorder is arrhythmia. In some embodiments, the arrhythmia is inherited or acquired. In some embodiments, the inherited arrhythmia is Catecholaminergic Polymorphic Ventricular Tachycardia (CPVT). In some embodiments, the acquired arrhythmia is a ventricular arrhythmia or a supraventricular arrhythmia. In some embodiments, the ventricular arrhythmia is ventricular tachycardia, ventricular fibrillation, or premature ventricular contraction. In some embodiments, the supraventricular arrhythmia is atrial fibrillation, atrial flutter, atrial tachycardia, premature atrial contraction, or paroxysmal supraventricular tachycardia. In some embodiments, the disorder is heart failure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. The patent or application file contains at least one drawing executed in color. Copies of this patent or patent application publication with color drawing(s) will be provided by the Office upon request and payment of the necessary fee. In the drawings:

FIGS. 1A-1F show MYBPC3 is present within dyads. FIG. 1A. Schematic depicting proximity proteomics strategy to identify proteins in dyads. AAV9 directed cardiomyocyte expression of a fusion protein between either Junctin (J) or Triadin (T) and BirA*, which catalyzes the formation of short-lived biotin free radicals. Junctin and Triadin and proteins that closely associate with RYR2 in cardiomyocyte dyads, which are the specialized Ca²⁺ release structures of these cells. FIG. 1B. Timeline of the experiment. AAV was delivered to neonatal mice. In the third week of life, biotin proximity labeling was induced by injection of biotin. Samples were collected at P28. Biotin-labeled proteins were isolated on immobilized streptavidin and analyzed by mass spectrometry. FIG. 1C. Localization of myc-tagged fusion proteins in cardiomyocytes, within heart sections. FIG. 1D. Higher magnification of showing that fusion proteins co-localize with CAV3 at T-tubules in dissociated cardiomyocytes. FIG. 1E. Input and streptavidin-bound proteins were visualized using streptavidin-HRP (biotinylated proteins, left) or a total protein stain (right). NC, negative control AAV (AAV-cTNT-GFP). FIG. 1F. Mass spectrometry analysis identified proteins in NC cardiomyocytes, and cardiomyocytes expressing BirA*-Triadin or BirA*-Junctin. The focus was on the set of proteins enriched in both the Triadin and Junctin fusion protein samples, and not the control samples (outlined region). MYBPC3 was among this set of proteins. Gene ontology terms enriched among the 177 proteins of interest (are shown to the right). These functional annotations were highly enriched for cardiomyocyte-related terms.

FIGS. 2A-2F show the subcellular localization of Mybpc3 and Mybpc3-derived peptides in cardiomyocytes. FIG. 2A. Domain structure of full length MYBPC3. Domains are labeled C0 to C10. FIG. 2B. Localization of endogenous C-terminal domain of MYBPC3 compared to RYR2 in wild-type cardiomyocytes (left). MYBPC3 protein was detected using a monoclonal antibody specific to the C10 domain (amino acids 1213-1229). This antibody did not display immunoreactivity to MYBPC3 null cardiomyocytes (right). C10 immunoreactivity co-localized with RYR2 at dyads. FIG. 2C. Co-localization of MYBPC3 and RYR2 by proximity ligation assay (PLA). The MYBPC3-C10 and RYR2 antibodies labeled proteins co-localized in situ, as determined by PLA signal (dots). Bar=10 μm. FIG. 2D. Quantification of PLA signal in samples stained with RYR2 antibody alone or in combination with the MYBPC3-C10 antibody. FIGS. 2E-2F. Localization of AAV-expressed, HA-tagged proteins. HA-tagged full length and MYBPC3 C-terminal peptides exhibited two distinct staining patterns, color coded red and blue. Full-length and the fragment encompassing domains C6-C10 (red, top) had a bifid immunostaining pattern fluorescence signal profile (FIG. 2F) consistent with predominant localization to the sarcomere A band. However, this pattern does not exclude that a subset of the proteins localizes to the dyad. The peptides encompassing C6-C8 and C6-C9, and the C10 domain alone, had a distinct fluorescent staining pattern and signal profile (blue, bottom) that was consistent with localization to dyads. Bar=10 μm.

FIGS. 3A-3D show MYBPC3 overexpression normalized Ca²⁺ handling in CPVT hiPSC-CMs. Human iPSCs from a patient with CPVT due to a heterozygous RYR2R4651I mutation were differentiated into cardiomyocytes (iPSC-CMs) and then transduced with adenovirus that expressed MYBPC3 or the control. FIGS. 3A-3B. Validation of Ad-HA-Mybpc3 mediated protein expression in iPSC-CMs. Western blotting (FIG. 3A) showed that Ad-HA-Mybpc3 induced ˜2.8-fold over-expression of full length MYBPC3. GAPDH was used as an internal control. The relative level of MYBPC3 compared to control iPSC-CMs is indicated by number above each lane. Protein expression was further confirmed by immunostaining iPSC-CMs using HA antibody. FIG. 3C. Confocal line scan images of Ca²⁺ signals from CPVT iPSC-CMs treated with control or Ad-hMYBPC3 adenovirus under normal or isoproterenol stimulation. FIG. 3D. Comparison of Ca²⁺ release event frequency, amplitude, FWHM (full width at half width) and FDHM (full duration at half maximum). Mann-Whitney test: ***, P<0.001.

FIGS. 4A-4H show FL-MYBPC3 overexpression normalized Ca²⁺ handling in adult CPVT (RYR2R176Q/+) cardiomyocytes and mice. FIG. 4A. Structure of AAV vector. GFP marks transduced cells. FIG. 4B. Heart sections of AAV-transduced cells. FIGS. 4C-4D. Western blot showing the overexpression (OE) of MYBPC3 and its quantification (FIG. 4D). FIGS. 4E-4F. Suppression of abnormal post-pacing Ca²⁺ waves in isolated CPVT (RYR2R176Q/+) adult cardiomyocytes by MYBPC3 overexpression. WT or CPVT mice were treated with indicated AAV. Cardiomyocytes were isolated from adult hearts and loaded with Ca²⁺ sensitive dye. Cardiomyocytes were paced (bold dashes), and then pacing was abruptly stopped. Post-pacing activity was recorded by confocal line scanning. Representative traces are shown. Comparison of post-pacing event frequency of RYR2-R176Q/+ cardiomyocytes (FIG. 4F) showed that these events were less frequent after MYBPC3 treatment. t-test: P<0.001. FIGS. 4G-4H. MYBPC3 overexpression reduced VT vulnerability in CPVT mice. Representative EKG traces are shown from AAV-GFP (control) and AAV-MYBPC3 treated CPVT mice. Pacing (bold dashes) with premature stimuli-initiated VT in GFP-treated but mice. The frequency of induced VT in RYR2-R176Q/+ mice was reduced by over-expression of full-length MYBPC3 (Fisher exact: P=0.0012; FIG. 4H) and became indistinguishable from WT mice. Numbers indicate number of mice with inducible VT and total mice.

FIGS. 5A-5E show efficacy of MYBPC3 fragments in suppressing VT in CPVT mice. FIGS. 5A-5B show in vivo testing of multiple different MYBPC3 C-terminal fragments for their activity in suppressing VT in CPVT mice. Neonatal mice were treated with 5.5×1010 vg/g of AAV expressing the indicated protein. Adult mice (8-16 weeks of age) were tested for contractile function (as shown in FIG. 5A) and VT vulnerability (as shown in FIG. 5B). FIG. 5A shows the effect of MYBPC3 peptides on heart function of RYR2R176Q/+ mice as determined by echocardiography. Although most fragments did not significantly affect heart function, the C6C9 and C6C7 peptides reduced heart contraction. One-way ANOVA with Dunnett's post-hoc test compared to GFP control treatment. Adjusted p-values are shown. Numbers within bars indicate number of mice per group. FIG. 5B shows the effect of MYBPC3 peptides on VT vulnerability of RYR2R176Q/+ mice. Mice underwent a graded protocol of programmed ventricular stimulation without β-agonist followed by stimulation with isoproterenol and then epinephrine plus caffeine. EP studies were performed blinded to treatment group. Sample sizes are indicated by numbers with the bars. Statistical significance was evaluated by the Fisher exact test compared to the GFP control group. Nominal p-values are shown above the bars. Those below the Bonferroni-corrected p-value threshold (0.05/8=0.0065) are marked with an asterisk. FIG. 5C shows the representative programmed ventricular stimulation of RYR2R176Q/+ mice treated with AAV-GFP or AAV-C6C10. The asterisked line indicates programmed ventricular stimulation. FIG. 5D shows representative Ca2+ tracing of RYR2S404R/WT human iPSC-CM treated with Ad-LacZ (control) or Ad-C6C10. Arrows highlight abnormal Ca2+ release events (aCREs). FIG. 5E shows quantification of frequency of aCREs. *, P<0.05.

FIG. 6 shows a schematic of a bimolecular fluorescence complementation assay (BiFC) that is used to map a minimal fragment of MYBPC3 that interacts with RYR2. The MYBPC3 fragments and RYR2 regions are each fused to half of a Venus fluorescent protein. When MYBPC3 and RYR2 bind, the two halves are brought into proximity and produce a fluorescent signal.

FIG. 7 shows the negative (RYR2) control for the BiFC experiment. RYR2 and SERCA2 are each fused to the N- and C-terminal halves of Venus (VN155 and VC155, respectively). There is no detectable Venus fluorescent signal, consistent with lack of RYR2-SERCA2 interaction.

FIG. 8 shows the positive (PLN) control for the BiFC experiment. PLN and SERCA2 are each fused to the N- and C-terminal halves of Venus (VN155 and VC155, respectively). There is bright Venus fluorescent signal, consistent with known PLN-SERCA2 interaction.

FIGS. 9A-9F shows regions of the MYBPC3 protein tested for binding to RYR2 using BiFC and results from tests. FIG. 9A shows regions of the MYBPC3 protein tested for binding to RYR2. FIG. 9B shows the design of the BiFC experiment. MYBPC3 fragments are fused to the C-terminal fragment of Venus (VC155), and RYR2 is fused to the N-terminal fragment of Venus (VN155). FIGS. 9C and 9D provides evidence that the C6-C8 region of MYBPC3 facilitates the interaction with RYR2. FIG. 9E shows by tiling deletion from C-terminus to N-terminus of the C6-C8 fragment that the C7-C8 is the major interacting domain with RYR2. FIG. 9F shows that deletion of either the C7 domain or the C8 domain does not completely abolish binding with RYR2 demonstrating that C7-C8 interacts robustly with RYR2.

FIG. 10 shows by immunostaining that non-interacting fragments of MYBCP3 and RYR2 are robustly expressed, excluding technical failure of expression as the reason for low Venus signal.

FIG. 11 shows that MYPBC3 fragments comprising C7-C8 fragments bind to RYR2 and that C7-C8 is the critical region for the interaction between MYPBC3 and RYR2.

FIG. 12 shows a summary schematic of the different MYPBC3 fragments tested and binding affinity to RYR2.

FIGS. 13A-13B show that the C7 fragment is sufficient for RYR2 binding and the predominant interacting domain with RYR2 in human (FIG. 13A) and mouse (FIG. 13B).

FIGS. 14A-14E show MYBPC3 is cleaved in vivo and that the two fragments of MYBPC3 bind predominantly to the Z-line or A-band. FIG. 14A shows the MYBPC3 construct used in FIGS. 14A-14E. The construct is MYBPC3 with a C-terminal Myc tag and a N-terminal HA tag. FIG. 14B shows how different cardiomyocytes in the same field of view have different staining patterns, Z-line pattern or A-band pattern. FIG. 14C shows that the C-terminus Myc tag has a predominantly Z-line pattern whereas the N-terminus HA tag has a predominantly A-band pattern. FIGS. 14D-14E show that N-terminal HA and C-terminal Myc have different sub-cellular location patterns as determined by electron microscopy.

FIG. 15 suggests that a fraction of MYPBC3 is internally cleaved to yield a smaller protein that includes its C-terminal domain. Cardiomyocyte lysates from wild type, wild-type+HA-MYBPC3-MYC, and MYBPC3 KO hearts were probed using HA or C10 (monoclonal Ab that recognizes the C-terminal most domain of MYBPC3) antibody.

FIG. 16A-16B shows that the C7-C8 fragment localized in a Z-line pattern in cardiomyocytes. FIG. 16A Mice were treated with AAV-cTnT-HA-C7C8-P2A-GFP. Heart sections were stained with HA and ACTN2 (a Z-line marker). Boxed area is enlarged to right. FIG. 16B shows the correlated presence between C7-C8 domain binding and sacromeric alpha actinin (SAA or ACTN2).

FIG. 17 shows MYBPC3 C6-C10 suppress abnormal Ca2+ release events in the CPVT RYR2-S404R mutant cells derived from human induced pluripotent stem cells differentiated into cardiomyocytes (iPSC-CMs).

### DETAILED DESCRIPTION OF CERTAIN EMBODIMENTS

CPVT (Catecholaminergic Polymorphic Ventricular Tachycardia) is a malignant inherited arrhythmia in which patients are at risk for lethal ventricular arrhythmias during exercise. CPVT is caused by mutations in cardiomyocyte Ca²⁺ handling genes. Over 60% of cases are caused by mutations in the gene RYR2 (ryanodine receptor type 2), which encodes the major intracellular Ca²⁺ release channel. We have discovered a novel interaction between the C-terminus of an endogenous cardiac protein and RYR2. Overexpression of this interacting domain suppressed aberrant RYR2 activity that is the root cause of arrhythmias in CPVT. This overexpression strategy normalized Ca²⁺ handling in human iPSC-derived cardiomyocytes, and suppressed arrhythmia in a mouse model of CPVT. Importantly, dysfunction of RYR2 is a final common pathway underlying diverse cardiac arrhythmias. Our findings on CPVT serve as a proof-of-concept. We believe that our therapeutic concept is likely applicable to other inherited and acquired arrhythmias.

The present disclosure, in some aspects, provides compositions and methods (e.g., gene therapy or protein therapy) for a disorder associated with abnormal RYR2 function. It was demonstrated herein that polypeptides comprising a C-terminal domain of Cardiac Myosin binding protein C (MYBPC3), or nucleic acids encoding such polypeptides are effective in treating arrhythmia. In some embodiments, the compositions and methods described herein can be used to treat arrhythmia or heart failure that are either inherited or acquired, including arterial fibrillation.

Accordingly, some aspects of the present disclosure provide methods of treating arrhythmia. In some embodiments, the method comprising administering to a subject in need thereof an effective amount of a polypeptide comprising a C-terminal domain of Cardiac Myosin binding protein C (MYBPC3). In some embodiments, the method comprising administering to a subject in need thereof an effective amount of a nucleic acid comprising a nucleotide sequence encoding a polypeptide comprising a C-terminal domain of MYBPC3.

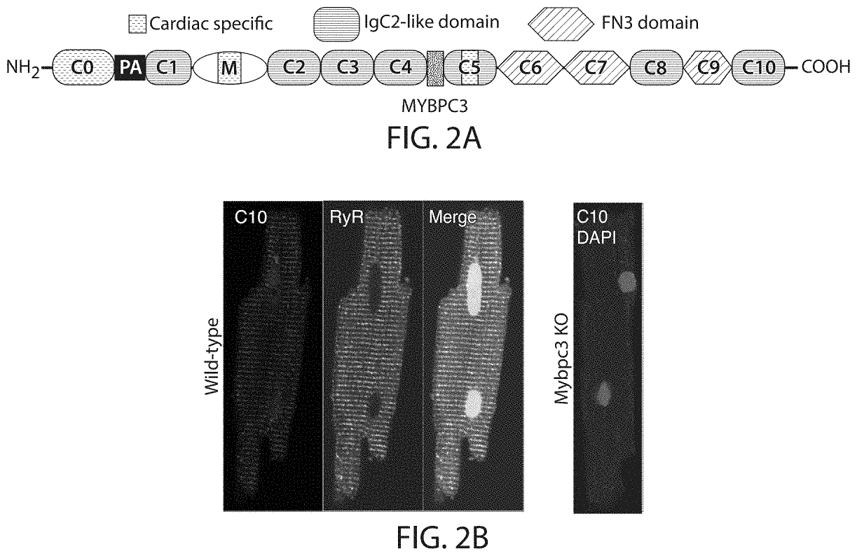

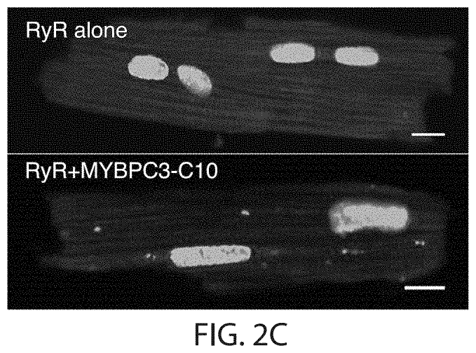

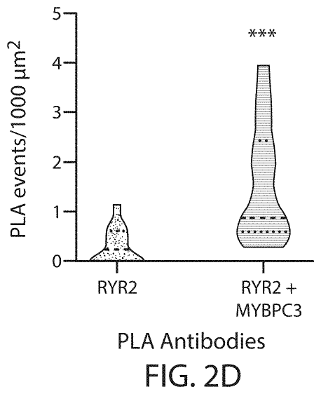

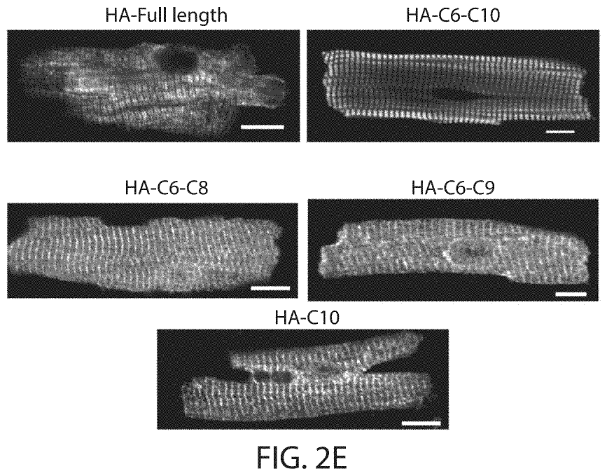

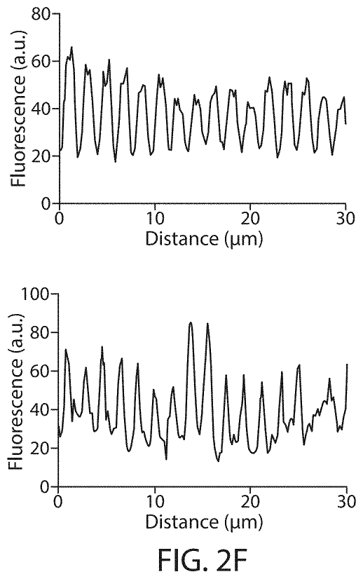

“Cardiac Myosin binding protein C (MYBPC3)” is found in cardiac muscle cells. In these cells, MYBPC3 is known to be associated with a structure called the sarcomere, which is the basic unit of muscle contraction. Sarcomeres are made up of thick and thin filaments. It was surprisingly found herein that, C-terminal domain fragments of the MYBPC3 protein localizes to dyads in the sarcomere, wherein the RYR2 protein is localized, while full-length MYBPC3 localizes to a different portion of the sarcomere. Human MYBPC3 protein sequence is provided under GenBank Accession No. NP_000247. Mouse MYBPC3 protein sequence is provided under GenBank Accession No. NP_032679.2. The domain structure of MYBPC3 is described in Sadayappan et al. (Biophys Rev. 2012 June; 4(2): 93-106, incorporated herein by references) and also illustrated in FIG. 2A.

In some embodiments, the polypeptide used in the methods described herein comprises a C-terminal domain (e.g., the C7-C8 domains as shown in FIG. 2A) of MYBPC3. In some embodiments, the polypeptide used in the methods described herein comprises C7 and C8 domains of MYBPC3. In some embodiments, the polypeptide used in the methods described herein consists of C7 and C8 domains of MYBPC3. In some embodiments, the polypeptide used in the methods described herein comprises the C7 domain of MYBPC3. In some embodiments, the polypeptide used in the methods described herein consists of the C7 domain of MYBPC3. In some embodiments, the polypeptide used in the methods described herein comprises the C8 domain of MYBPC3. In some embodiments, the polypeptide used in the methods described herein consists of the C8 domain of MYBPC3. In some embodiments, the polypeptide used in the methods described herein comprises C6, C7, C8, C9, and C10 domains of MYBPC3. In some embodiments, the polypeptide used in the methods described herein comprises C6, C7, C8, and C9 domains of MYBPC3. In some embodiments, the polypeptide used in the methods described herein comprises C6, C7, and C8 domains of MYBPC3. In some embodiments, the polypeptide used in the methods described herein comprises C6 and C7 domains of MYBPC3. In some embodiments, the polypeptide used in the methods described comprises a full-length MYBPC3. Examples of amino acid sequences of the polypeptides or nucleotide sequences encoding the polypeptides that may be used in the methods described herein are provided in Table 1.

In some embodiments, the polypeptide used in the methods described herein comprises the full-length mouse MYBPC3 of SEQ ID NO: 1, consists essentially of the full-length mouse MYBPC3 of SEQ ID NO: 1 or consists of the full-length mouse MYBPC3 of SEQ ID NO: 1. In some embodiments, the polypeptide used in the methods described herein comprises the mouse MYBPC3 C6-C7 (SEQ ID NO: 2), consists essentially of the mouse MYBPC3 C6-C7 (SEQ ID NO: 2) or consists of the mouse MYBPC3 C6-C7 (SEQ ID NO: 2). In some embodiments, the polypeptide used in the methods described herein comprises the mouse MYBPC3 C6-C8 (SEQ ID NO: 3), consists essentially of the mouse MYBPC3 C6-C8 (SEQ ID NO: 3) or consists of the mouse MYBPC3 C6-C8 (SEQ ID NO: 3). In some embodiments, the polypeptide used in the methods described herein comprises the mouse MYBPC3 C6-C9 (SEQ ID NO: 4), consists essentially of the mouse MYBPC3 C6-C9 (SEQ ID NO: 4) or consists of the mouse MYBPC3 C6-C9 (SEQ ID NO: 4). In some embodiments, the polypeptide used in the methods described herein comprises the mouse MYBPC3 C6-C10 (SEQ ID NO: 5), consists essentially of the mouse MYBPC3 C6-C10 (SEQ ID NO: 5) or consists of the mouse MYBPC3 C6-C10 (SEQ ID NO: 5). In some embodiments, the polypeptide used in the methods described herein comprises the mouse MYBPC3 C8-C10 (SEQ ID NO: 6), consists essentially of the mouse MYBPC3 C8-C10 (SEQ ID NO: 6) or consists of the mouse MYBPC3 C8-C10 (SEQ ID NO: 6). In some embodiments, the polypeptide used in the methods described herein comprises the mouse MYBPC3 C9-C10 (SEQ ID NO: 7), consists essentially of the mouse MYBPC3 C6-C7 (SEQ ID NO: 7) or consists of the mouse MYBPC3 C6-C7 (SEQ ID NO: 7). In some embodiments, the polypeptide used in the methods described herein comprises the mouse MYBPC3 C10 (SEQ ID NO: 8), consists essentially of the mouse MYBPC3 C10 (SEQ ID NO: 8) or consists of the mouse MYBPC3 C10 (SEQ ID NO: 8). In some embodiments, the polypeptide used in the methods described herein comprises the mouse MYBPC3 C7-C8 (SEQ ID NO: 59), consists essentially of the mouse MYBPC3 C7-C8 (SEQ ID NO: 59) or consists of the mouse MYBPC3 C7-C8 (SEQ ID NO: 59). In some embodiments, the polypeptide used in the methods described herein comprises the mouse MYBPC3 C7 (SEQ ID NO: 60), consists essentially of the mouse MYBPC3 C7 (SEQ ID NO: 60) or consists of the mouse MYBPC3 C7 (SEQ ID NO: 60). In some embodiments, the polypeptide used in the methods described herein comprises the mouse MYBPC3 C8 (SEQ ID NO: 61), consists essentially of the mouse MYBPC3 C8 (SEQ ID NO: 61) or consists of the mouse MYBPC3 C8 (SEQ ID NO: 61). In some embodiments, the polypeptide used in the methods described herein comprises the mouse MYBPC3 C7-C10 (SEQ ID NO: 62), consists essentially of the mouse MYBPC3 C7-C10 (SEQ ID NO: 62) or consists of the mouse MYBPC3 C7-C10 (SEQ ID NO: 62). In some embodiments, the polypeptide used in the methods described herein comprises the mouse MYBPC3 C6, C8-C10 (SEQ ID NO: 63), consists essentially of the mouse MYBPC3 C6, C8-C10 (SEQ ID NO: 63) or consists of the mouse MYBPC3 C6, C8-C10 (SEQ ID NO: 63). In some embodiments, the polypeptide used in the methods described herein comprises the mouse MYBPC3 C6-C7, C9-C10 (SEQ ID NO: 64), consists essentially of the mouse MYBPC3 C6-C7, C9-C10 (SEQ ID NO: 64) or consists of the mouse MYBPC3 C6-C7, C9-C10 (SEQ ID NO: 64).

In some embodiments, the polypeptide used in the methods described herein comprises the full length human MYBPC3 of SEQ ID NO: 9, consists essentially of the full length human MYBPC3 of SEQ ID NO: 9 or consists of the full length human MYBPC3 of SEQ ID NO: 9. In some embodiments, the polypeptide used in the methods described herein comprises the human MYBPC3 C6-C7 (SEQ ID NO: 10), consists essentially of the human MYBPC3 C6-C7 (SEQ ID NO: 10) or consists of the human MYBPC3 C6-C7 (SEQ ID NO: 10). In some embodiments, the polypeptide used in the methods described herein comprises the human MYBPC3 C6-C8 (SEQ ID NO: 11), consists essentially of the human MYBPC3 C6-C8 (SEQ ID NO: 11) or consists of the human MYBPC3 C6-C8 (SEQ ID NO: 11). In some embodiments, the polypeptide used in the methods described herein comprises the human MYBPC3 C6-C9 (SEQ ID NO: 12), consists essentially of the human MYBPC3 C6-C9 (SEQ ID NO: 12) or consists of the human MYBPC3 C6-C9 (SEQ ID NO: 12). In some embodiments, the polypeptide used in the methods described herein comprises the human MYBPC3 C6-C10 (SEQ ID NO: 13), consists essentially of the human MYBPC3 C6-C10 (SEQ ID NO: 13) or consists of the human MYBPC3 C6-C10 (SEQ ID NO: 13). In some embodiments, the polypeptide used in the methods described herein comprises the human MYBPC3 C8-C10 (SEQ ID NO: 14), consists essentially of the human MYBPC3 C8-C10 (SEQ ID NO: 14) or consists of the human MYBPC3 C8-C10 (SEQ ID NO: 14). In some embodiments, the polypeptide used in the methods described herein comprises the human MYBPC3 C9-C10 (SEQ ID NO: 15), consists essentially of the human MYBPC3 C6-C7 (SEQ ID NO: 15) or consists of the human MYBPC3 C6-C7 (SEQ ID NO: 15). In some embodiments, the polypeptide used in the methods described herein comprises the human MYBPC3 C10 (SEQ ID NO: 16), consists essentially of the human MYBPC3 C10 (SEQ ID NO: 16) or consists of the human MYBPC3 C10 (SEQ ID NO: 16). In some embodiments, the polypeptide used in the methods described herein comprises the human MYBPC3 C7-C8 (SEQ ID NO: 53) consists essentially of the human MYBPC3 C7-C8 (SEQ ID NO: 53) or consists of the human MYBPC3 C7-C8 (SEQ ID NO: 53). In some embodiments, the polypeptide used in the methods described herein comprises the human MYBPC3 C7 (SEQ ID NO: 54), consists essentially of the human MYBPC3 C7 (SEQ ID NO: 54) or consists of the human MYBPC3 C7 (SEQ ID NO: 54). In some embodiments, the polypeptide used in the methods described herein comprises the human MYBPC3 C8 (SEQ ID NO: 55), consists essentially of the human MYBPC3 C8 (SEQ ID NO: 55) or consists of the human MYBPC3 C8 (SEQ ID NO: 55). In some embodiments, the polypeptide used in the methods described herein comprises the human MYBPC3 C7-C10 (SEQ ID NO: 56), consists essentially of the human MYBPC3 C7-C10 (SEQ ID NO: 56) or consists of the human MYBPC3 C7-C10 (SEQ ID NO: 56). In some embodiments, the polypeptide used in the methods described herein comprises the human MYBPC3 C6, C8-C10 (SEQ ID NO: 57) consists essentially of the human MYBPC3 C6, C8-C10 (SEQ ID NO: 57) or consists of the human MYBPC3 C6, C8-C10 (SEQ ID NO: 57). In some embodiments, the polypeptide used in the methods described herein comprises the human MYPBC3 C6-C7, C9-C10 (SEQ ID NO: 58), consists essentially of the human MYBPC3 C6-C7, C9-C10 (SEQ ID NO: 58) or consists of the human MYBPC3 C6-C7, C9-C10 (SEQ ID NO: 58),In some embodiments, the polynucleotide used in the methods described herein comprises the full-length mouse MYBPC3 (SEQ ID NO: 17), consists essentially of the full-length mouse MYBPC3 (SEQ ID NO: 17) or consists of the full-length mouse MYBPC3 (SEQ ID NO: 17). In some embodiments, the polynucleotide used in the methods described herein comprises the mouse MYBPC3 C6-C7 (SEQ ID NO: 18), consists essentially of the mouse MYBPC3 C6-C7 (SEQ ID NO: 18) or consists of the mouse MYBPC3 C6-C7 (SEQ ID NO: 18). In some embodiments, the polynucleotide used in the methods described herein comprises the mouse MYBPC3 C6-C8 (SEQ ID NO: 19), consists essentially of the mouse MYBPC3 C6-C8 (SEQ ID NO: 19) or consists of the mouse MYBPC3 C6-C8 (SEQ ID NO: 19). In some embodiments, the polynucleotide used in the methods described herein comprises the mouse MYBPC3 C6-C9 (SEQ ID NO: 20), consists essentially of the mouse MYBPC3 C6-C9 (SEQ ID NO: 20) or consists of the mouse MYBPC3 C6-C9 (SEQ ID NO: 20). In some embodiments, the polynucleotide used in the methods described herein comprises the mouse MYBPC3 C6-C10 (SEQ ID NO: 21), consists essentially of the mouse MYBPC3 C6-C10 (SEQ ID NO: 21) or consists of the mouse MYBPC3 C6-C10 (SEQ ID NO: 21). In some embodiments, the polynucleotide used in the methods described herein comprises the mouse MYBPC3 C8-C10 (SEQ ID NO: 22), consists essentially of the mouse MYBPC3 C8-C10 (SEQ ID NO: 22) or consists of the mouse MYBPC3 C8-C10 (SEQ ID NO: 22). In some embodiments, the polynucleotide used in the methods described herein comprises the mouse MYBPC3 C9-C10 (SEQ ID NO: 23), consists essentially of the mouse MYBPC3 C6-C7 (SEQ ID NO: 23) or consists of the mouse MYBPC3 C6-C7 (SEQ ID NO: 23). In some embodiments, the polynucleotide used in the methods described herein comprises the mouse MYBPC3 C10 (SEQ ID NO: 24), consists essentially of the mouse MYBPC3 C10 (SEQ ID NO: 24) or consists of the mouse MYBPC3 C10 (SEQ ID NO: 24). In some embodiments, the polynucleotide used in the methods described herein comprises the mouse MYBPC3 C7-C8 (SEQ ID NO: 71), consists essentially of the mouse MYBPC3 C7-C8 (SEQ ID NO: 71) or consists of the mouse MYBPC3 C7-C8 (SEQ ID NO: 71). In some embodiments, the polynucleotide used in the methods described herein comprises the mouse MYBPC3 C7 (SEQ ID NO: 72), consists essentially of the mouse MYBPC3 C7 (SEQ ID NO: 72) or consists of the mouse MYBPC3 C7 (SEQ ID NO: 72). In some embodiments, the polynucleotide used in the methods described herein comprises the mouse MYBPC3 C8 (SEQ ID NO: 73), consists essentially of the mouse MYBPC3 C8 (SEQ ID NO: 73) or consists of the mouse MYBPC3 C8 (SEQ ID NO: 73). In some embodiments, the polynucleotide used in the methods described herein comprises the mouse MYBPC3 C7-C10 (SEQ ID NO: 74), consists essentially of the mouse MYBPC3 C7-C10 (SEQ ID NO: 74) or consists of the mouse MYBPC3 C7-C10 (SEQ ID NO: 74). In some embodiments, the polynucleotide used in the methods described herein comprises the mouse MYBPC3 C6, C8-C10 (SEQ ID NO: 75), consists essentially of the mouse MYBPC3 C6, C8-C10 (SEQ ID NO: 75) or consists of the mouse MYBPC3 C6, C8-C10 (SEQ ID NO: 75). In some embodiments, the polynucleotide used in the methods described herein comprises the mouse MYBPC3 C6-C7, C9-C10 (SEQ ID NO: 76), consists essentially of the mouse MYBPC3 C6-C7, C9-C10 (SEQ ID NO: 76) or consists of the mouse MYBPC3 C6-C7, C9-C10 (SEQ ID NO: 76).

In some embodiments, the polynucleotide used in the methods described herein comprises the full length human MYBPC3 (SEQ ID NO: 25), consists essentially of the full length human MYBPC3 (SEQ ID NO: 25) or consists of the full length human MYBPC3 (SEQ ID NO: 25). In some embodiments, the polynucleotide used in the methods described herein comprises the human MYBPC3 C6-C7 (SEQ ID NO: 26), consists essentially of the human MYBPC3 C6-C7 (SEQ ID NO: 26) or consists of the human MYBPC3 C6-C7 (SEQ ID NO: 26). In some embodiments, the polynucleotide used in the methods described herein comprises the human MYBPC3 C6-C8 (SEQ ID NO: 27), consists essentially of the human MYBPC3 C6-C8 (SEQ ID NO: 27) or consists of the human MYBPC3 C6-C8 (SEQ ID NO: 27). In some embodiments, the polynucleotide used in the methods described herein comprises the human MYBPC3 C6-C9 (SEQ ID NO: 28), consists essentially of the human MYBPC3 C6-C9 (SEQ ID NO: 28) or consists of the human MYBPC3 C6-C9 (SEQ ID NO: 28). In some embodiments, the polynucleotide used in the methods described herein comprises the human MYBPC3 C6-C10 (SEQ ID NO: 29), consists essentially of the human MYBPC3 C6-C10 (SEQ ID NO: 29) or consists of the human MYBPC3 C6-C10 (SEQ ID NO: 29). In some embodiments, the polynucleotide used in the methods described herein comprises the human MYBPC3 C8-C10 (SEQ ID NO: 30), consists essentially of the human MYBPC3 C8-C10 (SEQ ID NO: 30) or consists of the human MYBPC3 C8-C10 (SEQ ID NO: 30). In some embodiments, the polynucleotide used in the methods described herein comprises the human MYBPC3 C9-C10 (SEQ ID NO: 31), consists essentially of the human MYBPC3 C6-C7 (SEQ ID NO: 31) or consists of the human MYBPC3 C6-C7 (SEQ ID NO: 31). In some embodiments, the polynucleotide used in the methods described herein comprises the human MYBPC3 C10 (SEQ ID NO: 32), consists essentially of the human MYBPC3 C10 (SEQ ID NO: 32) or consists of the human MYBPC3 C10 (SEQ ID NO: 32). In some embodiments, the polynucleotide used in the methods described herein comprises the human MYBPC3 C7-C8 (SEQ ID NO: 65) consists essentially of the human MYBPC3 C7-C8 (SEQ ID NO: 65) or consists of the human MYBPC3 C7-C8 (SEQ ID NO: 65). In some embodiments, the polynucleotide used in the methods described herein comprises the human MYBPC3 C7 (SEQ ID NO: 66), consists essentially of the human MYBPC3 C7 (SEQ ID NO: 66) or consists of the human MYBPC3 C7 (SEQ ID NO: 66). In some embodiments, the polynucleotide used in the methods described herein comprises the human MYBPC3 C8 (SEQ ID NO: 67), consists essentially of the human MYBPC3 C8 (SEQ ID NO: 67) or consists of the human MYBPC3 C8 (SEQ ID NO: 67). In some embodiments, the polynucleotide used in the methods described herein comprises the human MYBPC3 C7-C10 (SEQ ID NO: 68), consists essentially of the human MYBPC3 C7-C10 (SEQ ID NO: 68) or consists of the human MYBPC3 C7-C10 (SEQ ID NO: 68). In some embodiments, the polynucleotide used in the methods described herein comprises the human MYBPC3 C6, C8-C10 (SEQ ID NO: 69) consists essentially of the human MYBPC3 C6, C8-C10 (SEQ ID NO: 69) or consists of the human MYBPC3 C6, C8-C10 (SEQ ID NO: 69). In some embodiments, the polynucleotide used in the methods described herein comprises the human MYPBC3 C6-C7, C9-C10 (SEQ ID NO: 70), consists essentially of the human MYBPC3 C6-C7, C9-C10 (SEQ ID NO: 70) or consists of the human MYBPC3 C6-C7, C9-C10 (SEQ ID NO: 70).

TABLE 1
MYBPC3 polypeptides

| Polypeptide | DNA Sequence | Amino Acid Sequence |
|---|---|---|
| Mouse full- | PGVTVLKMPEPGKKP | CCTGGTGTGACTGTTCTCAAGATGCCGGAGCCAGGGAAGAAACC |
| length | VSAFNKKPRSAEVTAG | AGTGTCAGCCTTCAACAAGAAGCCAAGGTCAGCGGAGGTGACCG |
| MYBPC3 | SAAVFEAETERSGVKV | CTGGCAGTGCTGCCGTGTTCGAGGCTGAGACGGAGCGGTCAGGC |
| | RWQRDGSDITANDKY | GTGAAGGTGCGGTGGCAGCGGGATGGCAGCGACATCACCGCCAA |
| | GLAAEGKRHTLTVRD | TGACAAGTATGGTTTGGCAGCAGAGGGCAAGCGACACACACTGA |
| | ASPDDQGSYAVIAGSS | CAGTGCGGGATGCGAGCCCTGATGACCAGGGTTCCTACGCGGTC |
| | KVKFDLKVTEPAPPEK | ATTGCAGGCTCCTCAAAGGTCAAGTTTGACCTCAAGGTCACAGAG |
| | AESEVAPGAPKEVPAP | CCAGCCCCTCCAGAGAAGGCAGAATCTGAAGTTGCTCCAGGAGC |
| | ATELEESVSSPEGSVSV | CCCCAAAGAAGTCCCTGCTCCAGCCACTGAGTTGGAAGAAAGTG |
| | TQDGSAAEHQGAPDD | TCTCAAGTCCTGAAGGGTCAGTCTCGGTAACCCAGGATGGCTCAG |
| | PIGLFLMRPQDGEVTV | CTGCAGAGCATCAGGGAGCCCCTGATGACCCTATTGGCCTCTTTC |
| | GGSIVFSARVAGASLL | TGATGCGACCACAGGATGGTGAGGTGACCGTGGGCGGCAGCATT |
| | KPPVVKWFKGKWVD | GTCTTCTCAGCCCGAGTGGCTGGGGCCAGCCTCCTGAAACCGCCT |
| | LSSKVGQHLQLHDSY | GTGGTCAAGTGGTTCAAGGGCAAGTGGGTGGACCTGAGCAGCAA |
| | DRASKVYLFELHITDA | AGTGGGCCAGCACCTGCAGCTGCATGACAGCTATGACAGAGCCA |
| | QTTSAGGYRCEVSTK | GCAAGGTCTACTTGTTTGAGTTGCACATCACAGATGCTCAGACCA |
| | DKFDSCNFNLTVHEAI | CTTCTGCTGGGGGCTACCGCTGTGAGGTGTCTACCAAGGACAAAT |
| | GSGDLDLRSAFRRTSL | TTGACAGCTGTAACTTCAACCTCACTGTCCATGAGGCCATTGGTT |
| | AGAGRRTSDSHEDAG | CTGGAGACCTGGACCTCAGATCAGCTTTCCGACGCACGAGCCTGG |
| | TLDFSSLLKKRDSFRR | CGGGAGCAGGTCGGAGAACCAGTGACAGCCATGAAGATGCTGGG |
| | DSKLEAPAEEDVWEIL | ACTCTGGACTTTAGTTCCCTGCTGAAGAAGAGAGACAGTTTCCGG |
| | RQAPPSEYERIAFQHG | AGGGACTCAAAGCTGGAGGCACCTGCTGAAGAAGACGTGTGGGA |
| | VTDLRGMLKRLKGMK | GATCCTGAGACAGGCACCGCCGTCAGAATATGAGCGCATCGCCT |
| | QDEKKSTAFQKKLEPA | TCCAGCACGGAGTCACAGACCTTCGAGGCATGCTGAAGAGGCTC |
| | YQVNKGHKIRLTVEL | AAGGGCATGAAGCAGGATGAAAAGAAGAGCACAGCCTTTCAGA |
| | ADPDAEVKWLKNGQE | AGAAGCTGGAGCCTGCCTACCAGGTAAACAAGGGCCACAAGATT |
| | IQMSGSKYIFESVGAK | CGGCTTACTGTGGAACTGGCTGATCCGGACGCCGAAGTCAAGTG |
| | RTLTISQCSLADDAAY | GCTTAAGAATGGACAGGAGATCCAGATGAGTGGCAGCAAGTACA |
| | QCVVGGEKCSTELFV | TCTTCGAGTCCGTCGGTGCCAAGCGCACCCTGACCATCAGCCAGT |
| | KEPPVLITRSLEDQLV | GCTCACTGGCTGACGACGCAGCCTACCAGTGTGTGGTGGGGGGC |
| | MVGQRVEFECEVSEE | GAGAAGTGCAGCACGGAGCTCTTTGTCAAAGAGCCCCCGGTGCT |
| | GAQVKWLKDGVELTR | GATCACTCGGTCCCTGGAAGACCAGCTGGTGATGGTGGGTCAGC |
| | EETFKYRFKKDGRKH | GGGTGGAGTTTGAGTGTGAGGTCTCAGAAGAAGGGGCCCAAGTC |
| | HLIINEATLEDAGHYA | AAATGGCTGAAGGATGGGGTTGAGCTGACACGTGAGGAGACCTT |
| | VRTSGGQSLAELIVQE | CAAATACCGGTTCAAGAAAGATGGGCGGAAACACCACTTGATCA |
| | KKLEVYQSIADLAVG | TCAATGAAGCAACCCTGGAGGATGCAGGACACTATGCAGTACGC |
| | AKDQAVFKCEVSDEN | ACAAGTGGAGGCCAGTCACTGGCTGAGCTCATTGTGCAAGAGAA |
| | VRGVWLKNGKELVPD | GAAGTTGGAGGTATACCAAAGCATCGCGGACCTGGCAGTGGGAG |
| | NRIKVSHIGRVHKLTID | CCAAGGACCAGGCTGTGTTTAAGTGTGAGGTTTCAGATGAGAAT |
| | DVTPADEADYSFVPEG | GTACGCGGCGTGTGGCTGAAGAATGGGAAGGAACTGGTGCCTGA |
| | FACNLSAKLHFMEVKI | CAACCGCATAAAGGTGTCCCATATAGGCCGGGTCCACAAACTGA |
| | DFVPRQEPPKIHLDCP | CCATTGACGATGTCACACCTGCTGATGAGGCTGACTACAGCTTTG |
| | GSTPDTIVVVAGNKLR | TCCCTGAAGGGTTTGCCTGCAACCTGTCTGCCAAGCTCCACTTCA |
| | LDVPISGDPAPTVVWQ | TGGAGGTCAAGATTGACTTTGTGCCTAGGCAGGAACCTCCCAAG |
| | KTVTQGKKASTGPHP | ATCCACTTGGATTGTCCCGGCAGCACACCAGACACCATTGTGGTT |
| | DAPEDAGADEEWVFD | GTTGCTGGGAACAAGTTACGCCTGGATGTCCCTATTTCTGGAGAC |
| | KKLLCETEGRVRVETT | CCTGCTCCCACTGTGGTCTGGCAGAAGACTGTAACACAGGGGAA |
| | KDRSVFTVEGAEKEDE | GAAGGCCTCAACTGGGCCACACCCTGATGCCCCAGAAGATGCTG |
| | GVYTVTVKNPVGEDQ | GTGCTGATGAGGAGTGGGTGTTTGATAAGAAGCTGTTGTGTGAG |
| | VNLTVKVIDVPDAPAA | ACTGAGGGCCGGGTCCGGGTGGAGACCACCAAAGACCGCAGCGT |
| | PKISNVGEDSCTVQWE | CTTTACAGTCGAAGGGGCAGAGAAGGAAGATGAAGGTGTCTACA |
| | PPAYDGGQPVLGYILE | CAGTCACAGTAAAGAACCCCGTGGGCGAGGACCAGGTCAACCTC |
| | RKKKKSYRWMRLNFD | ACAGTCAAGGTCATCGATGTCCCAGATGCTCCTGCGGCCCCTAAG |
| | LLRELSHEARRMIEGV | ATCAGCAACGTGGGCGAGGACTCCTGCACTGTGCAGTGGGAACC |
| | AYEMRVYAVNAVGM | GCCTGCCTATGATGGCGGGCAGCCGGTCCTGGGATACATCCTGGA |
| | SRPSPASQPFMPIGPPG | GCGCAAGAAGAAAAAGAGCTACAGGTGGATGAGGCTCAACTTTG |
| | EPTHLAVEDVSDTTVS | ATCTGCTGCGGGAGCTGAGCCACGAGGCGAGGCGCATGATCGAG |
| | LKWRPPERVGAGGLD | GGTGTAGCCTATGAGATGCGAGTCTACGCAGTCAATGCCGTGGG |
| | GYSVEYCQEGCSEWT | AATGTCCAGGCCCAGCCCTGCCTCTCAGCCCTTCATGCCTATTGG |
| | PALQGLTERTSMLVK | GCCCCCTGGCGAACCAACCCACTTGGCTGTGGAGGATGTGTCAG |
| | DLPTGARLLFRVRAHN | ACACCACTGTCTCACTCAAGTGGCGGCCCCCAGAGCGCGTGGGG |
| | VAGPGGPIVTKEPVTV | GCCGGTGGCCTGGACGGATACAGCGTGGAGTACTGCCAGGAGGG |
| | QEILQRPRLQLPRHLR | ATGCTCCGAGTGGACACCTGCTCTGCAGGGGCTGACAGAGCGCA |
| | QTIQKKVGEPVNLLIPF | CATCGATGCTGGTGAAGGACCTACCCACTGGGGCACGGCTGCTGT |
| | QGKPRPQVTWTKEGQ | TCCGAGTACGGGCACACAATGTGGCAGGTCCTGGAGGCCCTATC |
| | PLAGEEVSIRNSPTDTI | GTCACCAAGGAGCCTGTGACAGTGCAGGAGATACTGCAACGACC |
| | LFIRAARRTHSGTYQV | ACGGCTCCAACTGCCCAGACACCTGCGCCAGACCATCCAGAAGA |
| | TVRIENMEDKATLILQI | AAGTTGGGGAGCCTGTGAACCTCCTCATCCCTTTCCAGGGCAAAC |
| | VDKPSPPQDIRIVETW | CCCGGCCTCAGGTGACCTGGACCAAAGAGGGGCAGCCCCTGGCA |
| | GFNVALEWKPPQDDG | GGTGAGGAGGTGAGCATCCGGAACAGCCCCACAGACACGATCTT |
| | NTEIWGYTVQKADKK | GTTCATCCGAGCTGCCCGCCGCACCCACTCGGGCACCTACCAGGT |
| | TMEWFTVLEHYRRTH | GACAGTTCGCATTGAGAACATGGAGGACAAGGCAACGCTGATCC |
| | CVVSELIIGNGYYFRV | TGCAGATTGTGGACAAGCCAAGTCCTCCCCAGGATATCCGGATCG |
| | FSHNMVGSSDKAAAT | TTGAGACTTGGGGTTTCAATGTGGCTCTGGAGTGGAAGCCACCCC |
| | KEPVFIPRPGITYEPPK | AAGATGATGGCAATACAGAGATCTGGGGTTATACTGTACAGAAA |
| | YKALDFSEAPSFTQPL | GCTGACAAGAAGACCATGGAGTGGTTCACGGTTTTGGAACACTA |
| | ANRSIIAGYNAILCCA | CCGACGCACTCACTGTGTGGTATCAGAGCTTATCATTGGCAATGG |
| | VRGSPKPKISWFKNGL | CTACTACTTCCGGGTCTTCAGCCATAACATGGTGGGTTCCAGTGA |
| | DLGEDARFRMFCKQG | CAAAGCTGCCGCCACCAAGGAGCCAGTCTTTATTCCAAGACCAG |
| | VLTLEIRKPCPYDGGV | GCATCACATATGAGCCACCCAAATACAAGGCCCTGGACTTCTCTG |
| | YVCRATNLQGEAQCE | AGGCCCCAAGCTTCACCCAGCCCTTGGCAAATCGCTCCATCATTG |
| | CRLEVRVPQ | CAGGCTATAATGCCATCCTCTGCTGTGCTGTCCGAGGTAGTCCTA |
| | (SEQ ID NO: 1) | AGCCCAAGATTTCCTGGTTCAAGAATGGCCTGGATCTGGGAGAA |
| | | GATGCTCGCTTCCGCATGTTCTGCAAGCAGGGAGTATTGACCCTG |
| | | GAGATCAGGAAACCCTGCCCCTATGATGGTGGTGTCTATGTCTGC |
| | | AGGGCCACCAACTTGCAGGGCGAGGCACAGTGTGAGTGCCGCCT |
| | | GGAGGTGCGAGTTCCTCAG (SEQ ID NO: 17) |
| Mouse | APAAPKISNVGEDSCT | GCTCCTGCGGCCCCTAAGATCAGCAACGTGGGCGAGGACTCCTG |
| MYBPC3 | VQWEPPAYDGGQPVL | CACTGTGCAGTGGGAACCGCCTGCCTATGATGGCGGGCAGCCGG |
| C6-C7 | GYILERKKKKSYRWM | TCCTGGGATACATCCTGGAGCGCAAGAAGAAAAAGAGCTACAGG |
| | RLNFDLLRELSHEARR | TGGATGAGGCTCAACTTTGATCTGCTGCGGGAGCTGAGCCACGA |
| | MIEGVAYEMRVYAVN | GGCGAGGCGCATGATCGAGGGTGTAGCCTATGAGATGCGAGTCT |
| | AVGMSRPSPASQPFMP | ACGCAGTCAATGCCGTGGGAATGTCCAGGCCCAGCCCTGCCTCTC |
| | IGPPGEPTHLAVEDVS | AGCCCTTCATGCCTATTGGGCCCCCTGGCGAACCAACCCACTTGG |
| | DTTVSLKWRPPERVG | CTGTGGAGGATGTGTCAGACACCACTGTCTCACTCAAGTGGCGGC |
| | AGGLDGYSVEYCQEG | CCCCAGAGCGCGTGGGGGCCGGTGGCCTGGACGGATACAGCGTG |
| | CSEWTPALQGLTERTS | GAGTACTGCCAGGAGGGATGCTCCGAGTGGACACCTGCTCTGCA |
| | MLVKDLPTGARLLFR | GGGGCTGACAGAGCGCACATCGATGCTGGTGAAGGACCTACCCA |
| | VRAHNVAGPGGPIVT | CTGGGGCACGGCTGCTGTTCCGAGTACGGGCACACAATGTGGCA |
| | KEPVTVQEI | GGTCCTGGAGGCCCTATCGTCACCAAGGAGCCTGTGACAGTGCA |
| | (SEQ ID NO: 2) | GGAGATA (SEQ ID NO: 18) |
| Mouse | APAAPKISNVGEDSCT | GCTCCTGCGGCCCCTAAGATCAGCAACGTGGGCGAGGACTCCTG |
| MYBPC3 | VQWEPPAYDGGQPVL | CACTGTGCAGTGGGAACCGCCTGCCTATGATGGCGGGCAGCCGG |
| C6-C8 | GYILERKKKKSYRWM | TCCTGGGATACATCCTGGAGCGCAAGAAGAAAAAGAGCTACAGG |
| | RLNFDLLRELSHEARR | TGGATGAGGCTCAACTTTGATCTGCTGCGGGAGCTGAGCCACGA |
| | MIEGVAYEMRVYAVN | GGCGAGGCGCATGATCGAGGGTGTAGCCTATGAGATGCGAGTCT |
| | AVGMSRPSPASQPFMP | ACGCAGTCAATGCCGTGGGAATGTCCAGGCCCAGCCCTGCCTCTC |
| | IGPPGEPTHLAVEDVS | AGCCCTTCATGCCTATTGGGCCCCCTGGCGAACCAACCCACTTGG |
| | DTTVSLKWRPPERVG | CTGTGGAGGATGTGTCAGACACCACTGTCTCACTCAAGTGGCGGC |
| | AGGLDGYSVEYCQEG | CCCCAGAGCGCGTGGGGGCCGGTGGCCTGGACGGATACAGCGTG |
| | CSEWTPALQGLTERTS | GAGTACTGCCAGGAGGGATGCTCCGAGTGGACACCTGCTCTGCA |
| | MLVKDLPTGARLLFR | GGGGCTGACAGAGCGCACATCGATGCTGGTGAAGGACCTACCCA |
| | VRAHNVAGPGGPIVT | CTGGGGCACGGCTGCTGTTCCGAGTACGGGCACACAATGTGGCA |
| | KEPVTVQEILQRPRLQ | GGTCCTGGAGGCCCTATCGTCACCAAGGAGCCTGTGACAGTGCA |
| | LPRHLRQTIQKKVGEP | GGAGATACTGCAACGACCACGGCTCCAACTGCCCAGACACCTGC |
| | VNLLIPFQGKPRPQVT | GCCAGACCATCCAGAAGAAAGTTGGGGAGCCTGTGAACCTCCTC |
| | WTKEGQPLAGEEVSIR | ATCCCTTTCCAGGGCAAACCCCGGCCTCAGGTGACCTGGACCAAA |
| | NSPTDTILFIRAARRTH | GAGGGGCAGCCCCTGGCAGGTGAGGAGGTGAGCATCCGGAACAG |
| | SGTYQVTVRIENMED | CCCCACAGACACGATCTTGTTCATCCGAGCTGCCCGCCGCACCCA |
| | KATLILQIVDK | CTCGGGCACCTACCAGGTGACAGTTCGCATTGAGAACATGGAGG |
| | (SEQ ID NO: 3) | ACAAGGCAACGCTGATCCTGCAGATTGTGGACAAG (SEQ ID NO: |
| | | 19) |
| Mouse | APAAPKISNVGEDSCT | GCTCCTGCGGCCCCTAAGATCAGCAACGTGGGCGAGGACTCCTG |
| MYBPC3 | VQWEPPAYDGGQPVL | CACTGTGCAGTGGGAACCGCCTGCCTATGATGGCGGGCAGCCGG |
| C6-C9 | GYILERKKKKSYRWM | TCCTGGGATACATCCTGGAGCGCAAGAAGAAAAAGAGCTACAGG |
| | RLNFDLLRELSHEARR | TGGATGAGGCTCAACTTTGATCTGCTGCGGGAGCTGAGCCACGA |
| | MIEGVAYEMRVYAVN | GGCGAGGCGCATGATCGAGGGTGTAGCCTATGAGATGCGAGTCT |
| | AVGMSRPSPASQPFMP | ACGCAGTCAATGCCGTGGGAATGTCCAGGCCCAGCCCTGCCTCTC |
| | IGPPGEPTHLAVEDVS | AGCCCTTCATGCCTATTGGGCCCCCTGGCGAACCAACCCACTTGG |
| | DTTVSLKWRPPERVG | CTGTGGAGGATGTGTCAGACACCACTGTCTCACTCAAGTGGCGGC |
| | AGGLDGYSVEYCQEG | CCCCAGAGCGCGTGGGGGCCGGTGGCCTGGACGGATACAGCGTG |
| | CSEWTPALQGLTERTS | GAGTACTGCCAGGAGGGATGCTCCGAGTGGACACCTGCTCTGCA |
| | MLVKDLPTGARLLFR | GGGGCTGACAGAGCGCACATCGATGCTGGTGAAGGACCTACCCA |
| | VRAHNVAGPGGPIVT | CTGGGGCACGGCTGCTGTTCCGAGTACGGGCACACAATGTGGCA |
| | KEPVTVQEILQRPRLQ | GGTCCTGGAGGCCCTATCGTCACCAAGGAGCCTGTGACAGTGCA |
| | LPRHLRQTIQKKVGEP | GGAGATACTGCAACGACCACGGCTCCAACTGCCCAGACACCTGC |
| | VNLLIPFQGKPRPQVT | GCCAGACCATCCAGAAGAAAGTTGGGGAGCCTGTGAACCTCCTC |
| | WTKEGQPLAGEEVSIR | ATCCCTTTCCAGGGCAAACCCCGGCCTCAGGTGACCTGGACCAAA |
| | NSPTDTILFIRAARRTH | GAGGGGCAGCCCCTGGCAGGTGAGGAGGTGAGCATCCGGAACAG |
| | SGTYQVTVRIENMED | CCCCACAGACACGATCTTGTTCATCCGAGCTGCCCGCCGCACCCA |
| | KATLILQIVDKPSPPQD | CTCGGGCACCTACCAGGTGACAGTTCGCATTGAGAACATGGAGG |
| | IRIVETWGFNVALEWK | ACAAGGCAACGCTGATCCTGCAGATTGTGGACAAGCCAAGTCCT |
| | PPQDDGNTEIWGYTV | CCCCAGGATATCCGGATCGTTGAGACTTGGGGTTTCAATGTGGCT |
| | QKADKKTMEWFTVLE | CTGGAGTGGAAGCCACCCCAAGATGATGGCAATACAGAGATCTG |
| | HYRRTHCVVSELIIGN | GGGTTATACTGTACAGAAAGCTGACAAGAAGACCATGGAGTGGT |
| | GYYFRVFSHNMVGSS | TCACGGTTTTGGAACACTACCGACGCACTCACTGTGTGGTATCAG |
| | DKAAATKEPVFIPRP | AGCTTATCATTGGCAATGGCTACTACTTCCGGGTCTTCAGCCATA |
| | (SEQ ID NO: 4) | ACATGGTGGGTTCCAGTGACAAAGCTGCCGCCACCAAGGAGCCA |
| | | GTCTTTATTCCAAGACCA (SEQ ID NO: 20) |
| Mouse | APAAPKISNVGEDSCT | GCTCCTGCGGCCCCTAAGATCAGCAACGTGGGCGAGGACTCCTG |
| MYBPC3 | VQWEPPAYDGGQPVL | CACTGTGCAGTGGGAACCGCCTGCCTATGATGGCGGGCAGCCGG |
| C6-C10 | GYILERKKKKSYRWM | TCCTGGGATACATCCTGGAGCGCAAGAAGAAAAAGAGCTACAGG |
| | RLNFDLLRELSHEARR | TGGATGAGGCTCAACTTTGATCTGCTGCGGGAGCTGAGCCACGA |
| | MIEGVAYEMRVYAVN | GGCGAGGCGCATGATCGAGGGTGTAGCCTATGAGATGCGAGTCT |
| | AVGMSRPSPASQPFMP | ACGCAGTCAATGCCGTGGGAATGTCCAGGCCCAGCCCTGCCTCTC |
| | IGPPGEPTHLAVEDVS | AGCCCTTCATGCCTATTGGGCCCCCTGGCGAACCAACCCACTTGG |
| | DTTVSLKWRPPERVG | CTGTGGAGGATGTGTCAGACACCACTGTCTCACTCAAGTGGCGGC |
| | AGGLDGYSVEYCQEG | CCCCAGAGCGCGTGGGGGCCGGTGGCCTGGACGGATACAGCGTG |
| | CSEWTPALQGLTERTS | GAGTACTGCCAGGAGGGATGCTCCGAGTGGACACCTGCTCTGCA |
| | MLVKDLPTGARLLFR | GGGGCTGACAGAGCGCACATCGATGCTGGTGAAGGACCTACCCA |
| | VRAHNVAGPGGPIVT | CTGGGGCACGGCTGCTGTTCCGAGTACGGGCACACAATGTGGCA |
| | KEPVTVQEILQRPRLQ | GGTCCTGGAGGCCCTATCGTCACCAAGGAGCCTGTGACAGTGCA |
| | LPRHLRQTIQKKVGEP | GGAGATACTGCAACGACCACGGCTCCAACTGCCCAGACACCTGC |
| | VNLLIPFQGKPRPQVT | GCCAGACCATCCAGAAGAAAGTTGGGGAGCCTGTGAACCTCCTC |
| | WTKEGQPLAGEEVSIR | ATCCCTTTCCAGGGCAAACCCCGGCCTCAGGTGACCTGGACCAAA |
| | NSPTDTILFIRAARRTH | GAGGGGCAGCCCCTGGCAGGTGAGGAGGTGAGCATCCGGAACAG |
| | SGTYQVTVRIENMED | CCCCACAGACACGATCTTGTTCATCCGAGCTGCCCGCCGCACCCA |
| | KATLILQIVDKPSPPQD | CTCGGGCACCTACCAGGTGACAGTTCGCATTGAGAACATGGAGG |
| | IRIVETWGFNVALEWK | ACAAGGCAACGCTGATCCTGCAGATTGTGGACAAGCCAAGTCCT |
| | PPQDDGNTEIWGYTV | CCCCAGGATATCCGGATCGTTGAGACTTGGGGTTTCAATGTGGCT |
| | QKADKKTMEWFTVLE | CTGGAGTGGAAGCCACCCCAAGATGATGGCAATACAGAGATCTG |
| | HYRRTHCVVSELIIGN | GGGTTATACTGTACAGAAAGCTGACAAGAAGACCATGGAGTGGT |
| | GYYFRVFSHNMVGSS | TCACGGTTTTGGAACACTACCGACGCACTCACTGTGTGGTATCAG |
| | DKAAATKEPVFIPRPGI | AGCTTATCATTGGCAATGGCTACTACTTCCGGGTCTTCAGCCATA |
| | TYEPPKYKALDFSEAP | ACATGGTGGGTTCCAGTGACAAAGCTGCCGCCACCAAGGAGCCA |
| | SFTQPLANRSIIAGYNA | GTCTTTATTCCAAGACCAGGCATCACATATGAGCCACCCAAATAC |
| | ILCCAVRGSPKPKISWF | AAGGCCCTGGACTTCTCTGAGGCCCCAAGCTTCACCCAGCCCTTG |
| | KNGLDLGEDARFRMF | GCAAATCGCTCCATCATTGCAGGCTATAATGCCATCCTCTGCTGT |
| | CKQGVLTLEIRKPCPY | GCTGTCCGAGGTAGTCCTAAGCCCAAGATTTCCTGGTTCAAGAAT |
| | DGGVYVCRATNLQGE | GGCCTGGATCTGGGAGAAGATGCTCGCTTCCGCATGTTCTGCAAG |
| | AQCECRLEVRVPQ | CAGGGAGTATTGACCCTGGAGATCAGGAAACCCTGCCCCTATGA |
| | (SEQ ID NO: 5) | TGGTGGTGTCTATGTCTGCAGGGCCACCAACTTGCAGGGCGAGGC |
| | | ACAGTGTGAGTGCCGCCTGGAGGTGCGAGTTCCTCAG (SEQ ID |
| | | NO: 21) |
| Mouse | PRLQLPRHLRQTIQKK | CCACGGCTCCAACTGCCCAGACACCTGCGCCAGACCATCCAGAA |
| MYBPC3 | VGEPVNLLIPFQGKPR | GAAAGTTGGGGAGCCTGTGAACCTCCTCATCCCTTTCCAGGGCAA |
| C8-C10 | PQVTWTKEGQPLAGE | ACCCCGGCCTCAGGTGACCTGGACCAAAGAGGGGCAGCCCCTGG |
| | EVSIRNSPTDTILFIRAA | CAGGTGAGGAGGTGAGCATCCGGAACAGCCCCACAGACACGATC |
| | RRTHSGTYQVTVRIEN | TTGTTCATCCGAGCTGCCCGCCGCACCCACTCGGGCACCTACCAG |
| | MEDKATLILQIVDKPS | GTGACAGTTCGCATTGAGAACATGGAGGACAAGGCAACGCTGAT |
| | PPQDIRIVETWGFNVA | CCTGCAGATTGTGGACAAGCCAAGTCCTCCCCAGGATATCCGGAT |
| | LEWKPPQDDGNTEIW | CGTTGAGACTTGGGGTTTCAATGTGGCTCTGGAGTGGAAGCCACC |
| | GYTVQKADKKTMEW | CCAAGATGATGGCAATACAGAGATCTGGGGTTATACTGTACAGA |
| | FTVLEHYRRTHCVVSE | AAGCTGACAAGAAGACCATGGAGTGGTTCACGGTTTTGGAACAC |
| | LIIGNGYYFRVFSHNM | TACCGACGCACTCACTGTGTGGTATCAGAGCTTATCATTGGCAAT |
| | VGSSDKAAATKEPVFI | GGCTACTACTTCCGGGTCTTCAGCCATAACATGGTGGGTTCCAGT |
| | PRPGITYEPPKYKALD | GACAAAGCTGCCGCCACCAAGGAGCCAGTCTTTATTCCAAGACC |
| | FSEAPSFTQPLANRSII | AGGCATCACATATGAGCCACCCAAATACAAGGCCCTGGACTTCTC |
| | AGYNAILCCAVRGSPK | TGAGGCCCCAAGCTTCACCCAGCCCTTGGCAAATCGCTCCATCAT |
| | PKISWFKNGLDLGEDA | TGCAGGCTATAATGCCATCCTCTGCTGTGCTGTCCGAGGTAGTCC |
| | RFRMFCKQGVLTLEIR | TAAGCCCAAGATTTCCTGGTTCAAGAATGGCCTGGATCTGGGAGA |
| | KPCPYDGGVYVCRAT | AGATGCTCGCTTCCGCATGTTCTGCAAGCAGGGAGTATTGACCCT |
| | NLQGEAQCECRLEVR | GGAGATCAGGAAACCCTGCCCCTATGATGGTGGTGTCTATGTCTG |
| | VPQ | CAGGGCCACCAACTTGCAGGGCGAGGCACAGTGTGAGTGCCGCC |
| | (SEQ ID NO: 6) | TGGAGGTGCGAGTTCCTCAG (SEQ ID NO: 22) |
| Mouse | PPQDIRIVETWGFNVA | CCTCCCCAGGATATCCGGATCGTTGAGACTTGGGGTTTCAATGTG |
| MYBPC3 | LEWKPPQDDGNTEIW | GCTCTGGAGTGGAAGCCACCCCAAGATGATGGCAATACAGAGAT |
| C9-C10 | GYTVQKADKKTMEW | CTGGGGTTATACTGTACAGAAAGCTGACAAGAAGACCATGGAGT |
| | FTVLEHYRRTHCVVSE | GGTTCACGGTTTTGGAACACTACCGACGCACTCACTGTGTGGTAT |
| | LIIGNGYYFRVFSHNM | CAGAGCTTATCATTGGCAATGGCTACTACTTCCGGGTCTTCAGCC |
| | VGSSDKAAATKEPVFI | ATAACATGGTGGGTTCCAGTGACAAAGCTGCCGCCACCAAGGAG |
| | PRPGITYEPPKYKALD | CCAGTCTTTATTCCAAGACCAGGCATCACATATGAGCCACCCAAA |
| | FSEAPSFTQPLANRSII | TACAAGGCCCTGGACTTCTCTGAGGCCCCAAGCTTCACCCAGCCC |
| | AGYNAILCCAVRGSPK | TTGGCAAATCGCTCCATCATTGCAGGCTATAATGCCATCCTCTGC |
| | PKISWFKNGLDLGEDA | TGTGCTGTCCGAGGTAGTCCTAAGCCCAAGATTTCCTGGTTCAAG |
| | RFRMFCKQGVLTLEIR | AATGGCCTGGATCTGGGAGAAGATGCTCGCTTCCGCATGTTCTGC |
| | KPCPYDGGVYVCRAT | AAGCAGGGAGTATTGACCCTGGAGATCAGGAAACCCTGCCCCTA |
| | NLQGEAQCECRLEVR | TGATGGTGGTGTCTATGTCTGCAGGGCCACCAACTTGCAGGGCGA |
| | VPQ | GGCACAGTGTGAGTGCCGCCTGGAGGTGCGAGTTCCTCAG (SEQ |
| | (SEQ ID NO: 7) | ID NO: 23) |
| Mouse | PSFTQPLANRSIIAGYN | CCAAGCTTCACCCAGCCCTTGGCAAATCGCTCCATCATTGCAGGC |
| MYBPC3 | AILCCAVRGSPKPKIS | TATAATGCCATCCTCTGCTGTGCTGTCCGAGGTAGTCCTAAGCCC |
| C10 | WFKNGLDLGEDARFR | AAGATTTCCTGGTTCAAGAATGGCCTGGATCTGGGAGAAGATGCT |
| | MFCKQGVLTLEIRKPC | CGCTTCCGCATGTTCTGCAAGCAGGGAGTATTGACCCTGGAGATC |
| | PYDGGVYVCRATNLQ | AGGAAACCCTGCCCCTATGATGGTGGTGTCTATGTCTGCAGGGCC |
| | GEAQCECRLEVRVPQ | ACCAACTTGCAGGGCGAGGCACAGTGTGAGTGCCGCCTGGAGGT |
| | (SEQ ID NO: 8) | GCGAGTTCCTCAG (SEQ ID NO: 24) |
| Human full- | PEPGKKPVSAFSKKPR | CCTGAGCCGGGGAAGAAGCCAGTCTCAGCTTTTAGCAAGAAGCC |
| length | SVEVAAGSPAVFEAET | ACGGTCAGTGGAAGTGGCCGCAGGCAGCCCTGCCGTGTTCGAGG |
| MYBPC3 | ERAGVKVRWQRGGSD | CCGAGACAGAGCGGGCAGGAGTGAAGGTGCGCTGGCAGCGCGG |
| | ISASNKYGLATEGTRH | AGGCAGTGACATCAGCGCCAGCAACAAGTACGGCCTGGCCACAG |
| | TLTVREVGPADQGSY | AGGGCACACGGCATACGCTGACAGTGCGGGAAGTGGGCCCTGCC |
| | AVIAGSSKVKFDLKVI | GACCAGGGATCTTACGCAGTCATTGCTGGCTCCTCCAAGGTCAAG |
| | EAEKAEPMLAPAPAPA | TTCGACCTCAAGGTCATAGAGGCAGAGAAGGCAGAGCCCATGCT |
| | EATGAPGEAPAPAAEL | GGCCCCTGCCCCTGCCCCTGCTGAGGCCACTGGAGCCCCTGGAGA |
| | GESAPSPKGSSSAALN | AGCCCCGGCCCCAGCCGCTGAGCTGGGAGAAAGTGCCCCAAGTC |
| | GPTPGAPDDPIGLFVM | CCAAAGGGTCAAGCTCAGCAGCTCTCAATGGTCCTACCCCTGGAG |
| | RPQDGEVTVGGSITFS | CCCCCGATGACCCCATTGGCCTCTTCGTGATGCGGCCACAGGATG |
| | ARVAGASLLKPPVVK | GCGAGGTGACCGTGGGTGGCAGCATCACCTTCTCAGCCCGCGTG |
| | WFKGKWVDLSSKVG | GCCGGCGCCAGCCTCCTGAAGCCGCCTGTGGTCAAGTGGTTCAAG |
| | QHLQLHDSYDRASKV | GGCAAATGGGTGGACCTGAGCAGCAAGGTGGGCCAGCACCTGCA |
| | YLFELHITDAQPAFTG | GCTGCACGACAGCTACGACCGCGCCAGCAAGGTCTATCTGTTCGA |
| | SYRCEVSTKDKFDCSN | GCTGCACATCACCGATGCCCAGCCTGCCTTCACTGGCAGCTACCG |
| | FNLTVHEAMGTGDLD | CTGTGAGGTGTCCACCAAGGACAAATTTGACTGCTCCAACTTCAA |
| | LLSAFRRTSLAGGGRR | TCTCACTGTCCACGAGGCCATGGGCACCGGAGACCTGGACCTCCT |
| | ISDSHEDTGILDFSSLL | ATCAGCCTTCCGCCGCACGAGCCTGGCTGGAGGTGGTCGGCGGA |
| | KKRDSFRTPRDSKLEA | TCAGTGATAGCCATGAGGACACTGGGATTCTGGACTTCAGCTCAC |
| | PAEEDVWEILRQAPPS | TGCTGAAAAAGAGAGACAGTTTCCGGACCCCGAGGGACTCGAAG |
| | EYERIAFQYGVTDLRG | CTGGAGGCACCAGCAGAGGAGGACGTGTGGGAGATCCTACGGCA |
| | MLKRLKGMRRDEKKS | GGCACCCCCATCTGAGTACGAGCGCATCGCCTTCCAGTACGGCGT |
| | TAFQKKLEPAYQVSK | CACTGACCTGCGCGGCATGCTAAAGAGGCTCAAGGGCATGAGGC |
| | GHKIRLTVELADHDAE | GCGATGAGAAGAAGAGCACAGCCTTTCAGAAGAAGCTGGAGCCG |
| | VKWLKNGQEIQMSGS | GCCTACCAGGTGAGCAAAGGCCACAAGATCCGGCTGACCGTGGA |
| | KYIFESIGAKRTLTISQ | ACTGGCTGACCATGACGCTGAGGTCAAATGGCTCAAGAATGGCC |
| | CSLADDAAYQCVVGG | AGGAGATCCAGATGAGCGGCAGCAAGTACATCTTTGAGTCCATC |
| | EKCSTELFVKEPPVLIT | GGTGCCAAGCGTACCCTGACCATCAGCCAGTGCTCATTGGCGGAC |
| | RPLEDQLVMVGQRVE | GACGCAGCCTACCAGTGCGTGGTGGGTGGCGAGAAGTGTAGCAC |
| | FECEVSEEGAQVKWL | GGAGCTCTTTGTGAAAGAGCCCCCTGTGCTCATCACGCGCCCCTT |
| | KDGVELTREETFKYRF | GGAGGACCAGCTGGTGATGGTGGGGCAGCGGGTGGAGTTTGAGT |
| | KKDGQRHHLIINEAML | GTGAAGTATCGGAGGAGGGGGCGCAAGTCAAATGGCTGAAGGAC |
| | EDAGHYALCTSGGQA | GGGGTGGAGCTGACCCGGGAGGAGACCTTCAAATACCGGTTCAA |
| | LAELIVQEKKLEVYQS | GAAGGACGGGCAGAGACACCACCTGATCATCAACGAGGCCATGC |
| | IADLMVGAKDQAVFK | TGGAGGACGCGGGGCACTATGCACTGTGCACTAGCGGGGGCCAG |
| | CEVSDENVRGVWLKN | GCGCTGGCTGAGCTCATTGTGCAGGAAAAGAAGCTGGAGGTGTA |
| | GKELVPDSRIKVSHIG | CCAGAGCATCGCAGACCTGATGGTGGGCGCAAAGGACCAGGCGG |
| | RVHKLTIDDVTPADEA | TGTTCAAATGTGAGGTCTCAGATGAGAATGTTCGGGGTGTGTGGC |
| | DYSFVPEGFACNLSAK | TGAAGAATGGGAAGGAGCTGGTGCCCGACAGCCGCATAAAGGTG |
| | LHFMEVKIDFVPRQEP | TCCCACATCGGGCGGGTCCACAAACTGACCATTGACGACGTCAC |
| | PKIHLDCPGRIPDTIVV | ACCTGCCGACGAGGCTGACTACAGCTTTGTGCCCGAGGGCTTCGC |
| | VAGNKLRLDVPISGDP | CTGCAACCTGTCAGCCAAGCTCCACTTCATGGAGGTCAAGATTGA |
| | APTVIWQKAITQGNKA | CTTCGTACCCAGGCAGGAACCTCCCAAGATCCACCTGGACTGCCC |
| | PARPAPDAPEDTGDSD | AGGCCGCATACCAGACACCATTGTGGTTGTAGCTGGAAATAAGC |
| | EWVFDKKLLCETEGR | TACGTCTGGACGTCCCTATCTCTGGGGACCCTGCTCCCACTGTGA |
| | VRVETTKDRSIFTVEG | TCTGGCAGAAGGCTATCACGCAGGGGAATAAGGCCCCAGCCAGG |
| | AEKEDEGVYTVTVKN | CCAGCCCCAGATGCCCCAGAGGACACAGGTGACAGCGATGAGTG |
| | PVGEDQVNLTVKVID | GGTGTTTGACAAGAAGCTGCTGTGTGAGACCGAGGGCCGGGTCC |
| | VPDAPAAPKISNVGED | GCGTGGAGACCACCAAGGACCGCAGCATCTTCACGGTCGAGGGG |
| | SCTVQWEPPAYDGGQ | GCAGAGAAGGAAGATGAGGGCGTCTACACGGTCACAGTGAAGA |
| | PILGYILERKKKKSYR | ACCCTGTGGGCGAGGACCAGGTCAACCTCACAGTCAAGGTCATC |
| | WMRLNFDLIQELSHEA | GACGTGCCAGACGCACCTGCGGCCCCCAAGATCAGCAACGTGGG |
| | RRMIEGVVYEMRVYA | AGAGGACTCCTGCACAGTACAGTGGGAGCCGCCTGCCTACGATG |
| | VNAIGMSRPSPASQPF | GCGGGCAGCCCATCCTGGGCTACATCCTGGAGCGCAAGAAGAAG |
| | MPIGPPSEPTHLAVED | AAGAGCTACCGGTGGATGCGGCTGAACTTCGACCTGATTCAGGA |
| | VSDTTVSLKWRPPERV | GCTGAGTCATGAAGCGCGGCGCATGATCGAGGGCGTGGTGTACG |
| | GAGGLDGYSVEYCPE | AGATGCGCGTCTACGCGGTCAACGCCATCGGCATGTCCAGGCCC |
| | GCSEWVAALQGLTEH | AGCCCTGCCTCCCAGCCCTTCATGCCTATCGGTCCCCCCAGCGAA |
| | TSILVKDLPTGARLLFR | CCCACCCACCTGGCAGTAGAGGACGTCTCTGACACCACGGTCTCC |
| | VRAHNMAGPGAPVTT | CTCAAGTGGCGGCCCCCAGAGCGCGTGGGAGCAGGAGGCCTGGA |
| | TEPVTVQEILQRPRLQ | TGGCTACAGCGTGGAGTACTGCCCAGAGGGCTGCTCAGAGTGGG |
| | LPRHLRQTIQKKVGEP | TGGCTGCCCTGCAGGGGCTGACAGAGCACACATCGATACTGGTG |
| | VNLLIPFQGKPRPQVT | AAGGACCTGCCCACGGGGGCCCGGCTGCTTTTCCGAGTGCGGGC |
| | WTKEGQPLAGEEVSIR | ACACAATATGGCAGGGCCTGGAGCCCCTGTTACCACCACGGAGC |
| | NSPTDTILFIRAARRVH | CGGTGACAGTGCAGGAGATCCTGCAACGGCCACGGCTTCAGCTG |
| | SGTYQVTVRIENMED | CCCAGGCACCTGCGCCAGACCATTCAGAAGAAGGTCGGGGAGCC |
| | KATLVLQVVDKPSPPQ | TGTGAACCTTCTCATCCCTTTCCAGGGCAAGCCCCGGCCTCAGGT |
| | DLRVTDAWGLNVALE | GACCTGGACCAAAGAGGGGCAGCCCCTGGCAGGCGAGGAGGTG |
| | WKPPQDVGNTELWGY | AGCATCCGCAACAGCCCCACAGACACCATCCTGTTCATCCGGGCC |
| | TVQKADKKTMEWFTV | GCTCGCCGCGTGCATTCAGGCACTTACCAGGTGACGGTGCGCATT |
| | LEHYRRTHCVVPELIIG | GAGAACATGGAGGACAAGGCCACGCTGGTGCTGCAGGTTGTTGA |
| | NGYYFRVFSQNMVGF | CAAGCCAAGTCCTCCCCAGGATCTCCGGGTGACTGACGCCTGGG |
| | SDRAATTKEPVFIPRPG | GTCTTAATGTGGCTCTGGAGTGGAAGCCACCCCAGGATGTCGGCA |
| | ITYEPPNYKALDFSEAP | ACACGGAGCTCTGGGGGTACACAGTGCAGAAAGCCGACAAGAAG |
| | SFTQPLVNRSVIAGYT | ACCATGGAGTGGTTCACCGTCTTGGAGCATTACCGCCGCACCCAC |
| | AMLCCAVRGSPKPKIS | TGCGTGGTGCCAGAGCTCATCATTGGCAATGGCTACTACTTCCGC |
| | WFKNGLDLGEDARFR | GTCTTCAGCCAGAATATGGTTGGCTTTAGTGACAGAGCGGCCACC |
| | MFSKQGVLTLEIRKPC | ACCAAGGAGCCCGTCTTTATCCCCAGACCAGGCATCACCTATGAG |
| | PFDGGIYVCRATNLQG | CCACCCAACTATAAGGCCCTGGACTTCTCCGAGGCCCCAAGCTTC |
| | EARCECRLEVRVPQ | ACCCAGCCCCTGGTGAACCGCTCGGTCATCGCGGGCTACACTGCT |
| | (SEQ ID NO: 9) | ATGCTCTGCTGTGCTGTCCGGGGTAGCCCCAAGCCCAAGATTTCC |
| | | TGGTTCAAGAATGGCCTGGACCTGGGAGAAGACGCCCGCTTCCG |
| | | CATGTTCAGCAAGCAGGGAGTGTTGACTCTGGAGATTAGAAAGC |
| | | CCTGCCCCTTTGACGGGGGCATCTATGTCTGCAGGGCCACCAACT |
| | | TACAGGGCGAGGCACGGTGTGAGTGCCGCCTGGAGGTGCGAGTG |
| | | CCTCAG (SEQ ID NO: 25) |
| Human | APAAPKISNVGEDSCT | GCACCTGCGGCCCCCAAGATCAGCAACGTGGGAGAGGACTCCTG |
| MYBPC3 | VQWEPPAYDGGQPIL | CACAGTACAGTGGGAGCCGCCTGCCTACGATGGCGGGCAGCCCA |
| C6-C7 | GYILERKKKKSYRWM | TCCTGGGCTACATCCTGGAGCGCAAGAAGAAGAAGAGCTACCGG |
| | RLNFDLIQELSHEARR | TGGATGCGGCTGAACTTCGACCTGATTCAGGAGCTGAGTCATGAA |
| | MIEGVVYEMRVYAVN | GCGCGGCGCATGATCGAGGGCGTGGTGTACGAGATGCGCGTCTA |
| | AIGMSRPSPASQPFMPI | CGCGGTCAACGCCATCGGCATGTCCAGGCCCAGCCCTGCCTCCCA |
| | GPPSEPTHLAVEDVSD | GCCCTTCATGCCTATCGGTCCCCCCAGCGAACCCACCCACCTGGC |
| | TTVSLKWRPPERVGA | AGTAGAGGACGTCTCTGACACCACGGTCTCCCTCAAGTGGCGGCC |
| | GGLDGYSVEYCPEGCS | CCCAGAGCGCGTGGGAGCAGGAGGCCTGGATGGCTACAGCGTGG |
| | EWVAALQGLTEHTSIL | AGTACTGCCCAGAGGGCTGCTCAGAGTGGGTGGCTGCCCTGCAG |
| | VKDLPTGARLLFRVRA | GGGCTGACAGAGCACACATCGATACTGGTGAAGGACCTGCCCAC |
| | HNMAGPGAPVTTTEP | GGGGGCCCGGCTGCTTTTCCGAGTGCGGGCACACAATATGGCAG |
| | VTVQEI | GGCCTGGAGCCCCTGTTACCACCACGGAGCCGGTGACAGTGCAG |
| | (SEQ ID NO: 10) | GAGATC (SEQ ID NO: 26) |
| Human | APAAPKISNVGEDSCT | GCACCTGCGGCCCCCAAGATCAGCAACGTGGGAGAGGACTCCTG |
| MYBPC3 | VQWEPPAYDGGQPIL | CACAGTACAGTGGGAGCCGCCTGCCTACGATGGCGGGCAGCCCA |
| C6-C8 | GYILERKKKKSYRWM | TCCTGGGCTACATCCTGGAGCGCAAGAAGAAGAAGAGCTACCGG |
| | RLNFDLIQELSHEARR | TGGATGCGGCTGAACTTCGACCTGATTCAGGAGCTGAGTCATGAA |
| | MIEGVVYEMRVYAVN | GCGCGGCGCATGATCGAGGGCGTGGTGTACGAGATGCGCGTCTA |
| | AIGMSRPSPASQPFMPI | CGCGGTCAACGCCATCGGCATGTCCAGGCCCAGCCCTGCCTCCCA |
| | GPPSEPTHLAVEDVSD | GCCCTTCATGCCTATCGGTCCCCCCAGCGAACCCACCCACCTGGC |
| | TTVSLKWRPPERVGA | AGTAGAGGACGTCTCTGACACCACGGTCTCCCTCAAGTGGCGGCC |
| | GGLDGYSVEYCPEGCS | CCCAGAGCGCGTGGGAGCAGGAGGCCTGGATGGCTACAGCGTGG |
| | EWVAALQGLTEHTSIL | AGTACTGCCCAGAGGGCTGCTCAGAGTGGGTGGCTGCCCTGCAG |
| | VKDLPTGARLLFRVRA | GGGCTGACAGAGCACACATCGATACTGGTGAAGGACCTGCCCAC |
| | HNMAGPGAPVTTTEP | GGGGGCCCGGCTGCTTTTCCGAGTGCGGGCACACAATATGGCAG |
| | VTVQEILQRPRLQLPR | GGCCTGGAGCCCCTGTTACCACCACGGAGCCGGTGACAGTGCAG |
| | HLRQTIQKKVGEPVNL | GAGATCCTGCAACGGCCACGGCTTCAGCTGCCCAGGCACCTGCG |
| | LIPFQGKPRPQVTWTK | CCAGACCATTCAGAAGAAGGTCGGGGAGCCTGTGAACCTTCTCA |
| | EGQPLAGEEVSIRNSPT | TCCCTTTCCAGGGCAAGCCCCGGCCTCAGGTGACCTGGACCAAAG |
| | DTILFIRAARRVHSGT | AGGGGCAGCCCCTGGCAGGCGAGGAGGTGAGCATCCGCAACAGC |
| | YQVTVRIENMEDKAT | CCCACAGACACCATCCTGTTCATCCGGGCCGCTCGCCGCGTGCAT |
| | LVLQVVDK | TCAGGCACTTACCAGGTGACGGTGCGCATTGAGAACATGGAGGA |
| | (SEQ ID NO: 11) | CAAGGCCACGCTGGTGCTGCAGGTTGTTGACAAG |
| | | (SEQ ID NO: 27) |
| Human | APAAPKISNVGEDSCT | GCACCTGCGGCCCCCAAGATCAGCAACGTGGGAGAGGACTCCTG |
| MYBPC3 | VQWEPPAYDGGQPIL | CACAGTACAGTGGGAGCCGCCTGCCTACGATGGCGGGCAGCCCA |
| C6-C9 | GYILERKKKKSYRWM | TCCTGGGCTACATCCTGGAGCGCAAGAAGAAGAAGAGCTACCGG |
| | RLNFDLIQELSHEARR | TGGATGCGGCTGAACTTCGACCTGATTCAGGAGCTGAGTCATGAA |
| | MIEGVVYEMRVYAVN | GCGCGGCGCATGATCGAGGGCGTGGTGTACGAGATGCGCGTCTA |
| | AIGMSRPSPASQPFMPI | CGCGGTCAACGCCATCGGCATGTCCAGGCCCAGCCCTGCCTCCCA |
| | GPPSEPTHLAVEDVSD | GCCCTTCATGCCTATCGGTCCCCCCAGCGAACCCACCCACCTGGC |
| | TTVSLKWRPPERVGA | AGTAGAGGACGTCTCTGACACCACGGTCTCCCTCAAGTGGCGGCC |
| | GGLDGYSVEYCPEGCS | CCCAGAGCGCGTGGGAGCAGGAGGCCTGGATGGCTACAGCGTGG |
| | EWVAALQGLTEHTSIL | AGTACTGCCCAGAGGGCTGCTCAGAGTGGGTGGCTGCCCTGCAG |
| | VKDLPTGARLLFRVRA | GGGCTGACAGAGCACACATCGATACTGGTGAAGGACCTGCCCAC |
| | HNMAGPGAPVTTTEP | GGGGGCCCGGCTGCTTTTCCGAGTGCGGGCACACAATATGGCAG |
| | VTVQEILQRPRLQLPR | GGCCTGGAGCCCCTGTTACCACCACGGAGCCGGTGACAGTGCAG |
| | HLRQTIQKKVGEPVNL | GAGATCCTGCAACGGCCACGGCTTCAGCTGCCCAGGCACCTGCG |
| | LIPFQGKPRPQVTWTK | CCAGACCATTCAGAAGAAGGTCGGGGAGCCTGTGAACCTTCTCA |
| | EGQPLAGEEVSIRNSPT | TCCCTTTCCAGGGCAAGCCCCGGCCTCAGGTGACCTGGACCAAAG |
| | DTILFIRAARRVHSGT | AGGGGCAGCCCCTGGCAGGCGAGGAGGTGAGCATCCGCAACAGC |
| | YQVTVRIENMEDKAT | CCCACAGACACCATCCTGTTCATCCGGGCCGCTCGCCGCGTGCAT |
| | LVLQVVDKPSPPQDLR | TCAGGCACTTACCAGGTGACGGTGCGCATTGAGAACATGGAGGA |
| | VTDAWGLNVALEWK | CAAGGCCACGCTGGTGCTGCAGGTTGTTGACAAGCCAAGTCCTCC |
| | PPQDVGNTELWGYTV | CCAGGATCTCCGGGTGACTGACGCCTGGGGTCTTAATGTGGCTCT |
| | QKADKKTMEWFTVLE | GGAGTGGAAGCCACCCCAGGATGTCGGCAACACGGAGCTCTGGG |
| | HYRRTHCVVPELIIGN | GGTACACAGTGCAGAAAGCCGACAAGAAGACCATGGAGTGGTTC |
| | GYYFRVFSQNMVGFS | ACCGTCTTGGAGCATTACCGCCGCACCCACTGCGTGGTGCCAGAG |
| | DRAATTKEPVFIPRP | CTCATCATTGGCAATGGCTACTACTTCCGCGTCTTCAGCCAGAAT |
| | (SEQ ID NO: 12) | ATGGTTGGCTTTAGTGACAGAGCGGCCACCACCAAGGAGCCCGT |
| | | CTTTATCCCCAGACCA |
| | | (SEQ ID NO: 28) |
| Human | APAAPKISNVGEDSCT | GCACCTGCGGCCCCCAAGATCAGCAACGTGGGAGAGGACTCCTG |
| MYBPC3 | VQWEPPAYDGGQPIL | CACAGTACAGTGGGAGCCGCCTGCCTACGATGGCGGGCAGCCCA |
| C6-C10 | GYILERKKKKSYRWM | TCCTGGGCTACATCCTGGAGCGCAAGAAGAAGAAGAGCTACCGG |
| | RLNFDLIQELSHEARR | TGGATGCGGCTGAACTTCGACCTGATTCAGGAGCTGAGTCATGAA |
| | MIEGVVYEMRVYAVN | GCGCGGCGCATGATCGAGGGCGTGGTGTACGAGATGCGCGTCTA |
| | AIGMSRPSPASQPFMPI | CGCGGTCAACGCCATCGGCATGTCCAGGCCCAGCCCTGCCTCCCA |
| | GPPSEPTHLAVEDVSD | GCCCTTCATGCCTATCGGTCCCCCCAGCGAACCCACCCACCTGGC |
| | TTVSLKWRPPERVGA | AGTAGAGGACGTCTCTGACACCACGGTCTCCCTCAAGTGGCGGCC |
| | GGLDGYSVEYCPEGCS | CCCAGAGCGCGTGGGAGCAGGAGGCCTGGATGGCTACAGCGTGG |
| | EWVAALQGLTEHTSIL | AGTACTGCCCAGAGGGCTGCTCAGAGTGGGTGGCTGCCCTGCAG |
| | VKDLPTGARLLFRVRA | GGGCTGACAGAGCACACATCGATACTGGTGAAGGACCTGCCCAC |
| | HNMAGPGAPVTTTEP | GGGGGCCCGGCTGCTTTTCCGAGTGCGGGCACACAATATGGCAG |
| | VTVQEILQRPRLQLPR | GGCCTGGAGCCCCTGTTACCACCACGGAGCCGGTGACAGTGCAG |
| | HLRQTIQKKVGEPVNL | GAGATCCTGCAACGGCCACGGCTTCAGCTGCCCAGGCACCTGCG |
| | LIPFQGKPRPQVTWTK | CCAGACCATTCAGAAGAAGGTCGGGGAGCCTGTGAACCTTCTCA |
| | EGQPLAGEEVSIRNSPT | TCCCTTTCCAGGGCAAGCCCCGGCCTCAGGTGACCTGGACCAAAG |
| | DTILFIRAARRVHSGT | AGGGGCAGCCCCTGGCAGGCGAGGAGGTGAGCATCCGCAACAGC |
| | YQVTVRIENMEDKAT | CCCACAGACACCATCCTGTTCATCCGGGCCGCTCGCCGCGTGCAT |
| | LVLQVVDKPSPPQDLR | TCAGGCACTTACCAGGTGACGGTGCGCATTGAGAACATGGAGGA |
| | VTDAWGLNVALEWK | CAAGGCCACGCTGGTGCTGCAGGTTGTTGACAAGCCAAGTCCTCC |
| | PPQDVGNTELWGYTV | CCAGGATCTCCGGGTGACTGACGCCTGGGGTCTTAATGTGGCTCT |
| | QKADKKTMEWFTVLE | GGAGTGGAAGCCACCCCAGGATGTCGGCAACACGGAGCTCTGGG |
| | HYRRTHCVVPELIIGN | GGTACACAGTGCAGAAAGCCGACAAGAAGACCATGGAGTGGTTC |
| | GYYFRVFSQNMVGFS | ACCGTCTTGGAGCATTACCGCCGCACCCACTGCGTGGTGCCAGAG |
| | DRAATTKEPVFIPRPGI | CTCATCATTGGCAATGGCTACTACTTCCGCGTCTTCAGCCAGAAT |
| | TYEPPNYKALDFSEAP | ATGGTTGGCTTTAGTGACAGAGCGGCCACCACCAAGGAGCCCGT |
| | SFTQPLVNRSVIAGYT | CTTTATCCCCAGACCAGGCATCACCTATGAGCCACCCAACTATAA |
| | AMLCCAVRGSPKPKIS | GGCCCTGGACTTCTCCGAGGCCCCAAGCTTCACCCAGCCCCTGGT |
| | WFKNGLDLGEDARFR | GAACCGCTCGGTCATCGCGGGCTACACTGCTATGCTCTGCTGTGC |
| | MFSKQGVLTLEIRKPC | TGTCCGGGGTAGCCCCAAGCCCAAGATTTCCTGGTTCAAGAATGG |
| | PFDGGIYVCRATNLQG | CCTGGACCTGGGAGAAGACGCCCGCTTCCGCATGTTCAGCAAGC |
| | EARCECRLEVRVPQ | AGGGAGTGTTGACTCTGGAGATTAGAAAGCCCTGCCCCTTTGACG |
| | (SEQ ID NO: 13) | GGGGCATCTATGTCTGCAGGGCCACCAACTTACAGGGCGAGGCA |
| | | CGGTGTGAGTGCCGCCTGGAGGTGCGAGTGCCTCAG |
| | | (SEQ ID NO: 29) |
| Human | PRLQLPRHLRQTIQKK | CCACGGCTTCAGCTGCCCAGGCACCTGCGCCAGACCATTCAGAA |
| MYBPC3 | VGEPVNLLIPFQGKPR | GAAGGTCGGGGAGCCTGTGAACCTTCTCATCCCTTTCCAGGGCAA |
| C8-C10 | PQVTWTKEGQPLAGE | GCCCCGGCCTCAGGTGACCTGGACCAAAGAGGGGCAGCCCCTGG |
| | EVSIRNSPTDTILFIRAA | CAGGCGAGGAGGTGAGCATCCGCAACAGCCCCACAGACACCATC |
| | RRVHSGTYQVTVRIEN | CTGTTCATCCGGGCCGCTCGCCGCGTGCATTCAGGCACTTACCAG |
| | MEDKATLVLQVVDKP | GTGACGGTGCGCATTGAGAACATGGAGGACAAGGCCACGCTGGT |
| | SPPQDLRVTDAWGLN | GCTGCAGGTTGTTGACAAGCCAAGTCCTCCCCAGGATCTCCGGGT |
| | VALEWKPPQDVGNTE | GACTGACGCCTGGGGTCTTAATGTGGCTCTGGAGTGGAAGCCACC |
| | LWGYTVQKADKKTM | CCAGGATGTCGGCAACACGGAGCTCTGGGGGTACACAGTGCAGA |
| | EWFTVLEHYRRTHCV | AAGCCGACAAGAAGACCATGGAGTGGTTCACCGTCTTGGAGCAT |
| | VPELIIGNGYYFRVFSQ | TACCGCCGCACCCACTGCGTGGTGCCAGAGCTCATCATTGGCAAT |
| | NMVGFSDRAATTKEP | GGCTACTACTTCCGCGTCTTCAGCCAGAATATGGTTGGCTTTAGT |
| | VFIPRPGITYEPPNYKA | GACAGAGCGGCCACCACCAAGGAGCCCGTCTTTATCCCCAGACC |
| | LDFSEAPSFTQPLVNRS | AGGCATCACCTATGAGCCACCCAACTATAAGGCCCTGGACTTCTC |
| | VIAGYTAMLCCAVRG | CGAGGCCCCAAGCTTCACCCAGCCCCTGGTGAACCGCTCGGTCAT |
| | SPKPKISWFKNGLDLG | CGCGGGCTACACTGCTATGCTCTGCTGTGCTGTCCGGGGTAGCCC |
| | EDARFRMFSKQGVLT | CAAGCCCAAGATTTCCTGGTTCAAGAATGGCCTGGACCTGGGAG |
| | LEIRKPCPFDGGIYVCR | AAGACGCCCGCTTCCGCATGTTCAGCAAGCAGGGAGTGTTGACTC |
| | ATNLQGEARCECRLE | TGGAGATTAGAAAGCCCTGCCCCTTTGACGGGGGCATCTATGTCT |
| | VRVPQ | GCAGGGCCACCAACTTACAGGGCGAGGCACGGTGTGAGTGCCGC |
| | (SEQ ID NO: 14) | CTGGAGGTGCGAGTGCCTCAG |
| | | (SEQ ID NO: 30) |
| Human | PPQDLRVTDAWGLNV | CCTCCCCAGGATCTCCGGGTGACTGACGCCTGGGGTCTTAATGTG |
| MYBPC3 | ALEWKPPQDVGNTEL | GCTCTGGAGTGGAAGCCACCCCAGGATGTCGGCAACACGGAGCT |
| C9-C10 | WGYTVQKADKKTME | CTGGGGGTACACAGTGCAGAAAGCCGACAAGAAGACCATGGAGT |
| | WFTVLEHYRRTHCVV | GGTTCACCGTCTTGGAGCATTACCGCCGCACCCACTGCGTGGTGC |
| | PELIIGNGYYFRVFSQN | CAGAGCTCATCATTGGCAATGGCTACTACTTCCGCGTCTTCAGCC |
| | MVGFSDRAATTKEPV | AGAATATGGTTGGCTTTAGTGACAGAGCGGCCACCACCAAGGAG |
| | FIPRPGITYEPPNYKAL | CCCGTCTTTATCCCCAGACCAGGCATCACCTATGAGCCACCCAAC |
| | DFSEAPSFTQPLVNRS | TATAAGGCCCTGGACTTCTCCGAGGCCCCAAGCTTCACCCAGCCC |
| | VIAGYTAMLCCAVRG | CTGGTGAACCGCTCGGTCATCGCGGGCTACACTGCTATGCTCTGC |
| | SPKPKISWFKNGLDLG | TGTGCTGTCCGGGGTAGCCCCAAGCCCAAGATTTCCTGGTTCAAG |
| | EDARFRMFSKQGVLT | AATGGCCTGGACCTGGGAGAAGACGCCCGCTTCCGCATGTTCAG |
| | LEIRKPCPFDGGIYVCR | CAAGCAGGGAGTGTTGACTCTGGAGATTAGAAAGCCCTGCCCCTT |
| | ATNLQGEARCECRLE | TGACGGGGGCATCTATGTCTGCAGGGCCACCAACTTACAGGGCG |
| | VRVPQ | AGGCACGGTGTGAGTGCCGCCTGGAGGTGCGAGTGCCTCAG |
| | (SEQ ID NO: 15) | (SEQ ID NO: 31) |
| Human | PSFTQPLVNRSVIAGY | CCAAGCTTCACCCAGCCCCTGGTGAACCGCTCGGTCATCGCGGGC |
| MYBPC3 | TAMLCCAVRGSPKPKI | TACACTGCTATGCTCTGCTGTGCTGTCCGGGGTAGCCCCAAGCCC |
| C10 | SWFKNGLDLGEDARF | AAGATTTCCTGGTTCAAGAATGGCCTGGACCTGGGAGAAGACGC |
| | RMFSKQGVLTLEIRKP | CCGCTTCCGCATGTTCAGCAAGCAGGGAGTGTTGACTCTGGAGAT |
| | CPFDGGIYVCRATNLQ | TAGAAAGCCCTGCCCCTTTGACGGGGGCATCTATGTCTGCAGGGC |
| | GEARCECRLEVRVPQ | CACCAACTTACAGGGCGAGGCACGGTGTGAGTGCCGCCTGGAGG |
| | (SEQ ID NO: 16) | TGCGAGTGCCTCAG |
| | | (SEQ ID NO: 32) |
| Human | PPSEPTHLAVEDVSDT | CCCCCCAGCGAACCCACCCACCUGGCAGUAGAGGACGUCUCUGA |
| MYBPC3 | TVSLKWRPPERVGAG | CACCACGGUCUCCCUCAAGUGGCGGCCCCCAGAGCGCGUGGGA |
| C7-C8 | GLDGYSVEYCPEGCSE | GCAGGAGGCCUGGAUGGCUACAGCGUGGAGUACUGCCCAGAGG |
| | WVAALQGLTEHTSILV | GCUGCUCAGAGUGGGUGGCUGCCCUGCAGGGGCUGACAGAGCA |
| | KDLPTGARLLFRVRAH | CACAUCGAUACUGGUGAAGGACCUGCCCACGGGGGCCCGGCUG |
| | NMAGPGAPVTTTEPV | CUUUUCCGAGUGCGGGCACACAAUAUGGCAGGGCCUGGAGCCC |
| | TVQEILQRPRLQLPRH | CUGUUACCACCACGGAGCCGGUGACAGUGCAGGAGAUCCUGCA |
| | LRQTIQKKVGEPVNLL | ACGGCCACGGCUUCAGCUGCCCAGGCACCUGCGCCAGACCAUUC |
| | IPFQGKPRPQVTWTKE | AGAAGAAGGUCGGGGAGCCUGUGAACCUUCUCAUCCCUUUCCA |
| | GQPLAGEEVSIRNSPT | GGGCAAGCCCCGGCCUCAGGUGACCUGGACCAAAGAGGGGCAG |
| | DTILFIRAARRVHSGT | CCCCUGGCAGGCGAGGAGGUGAGCAUCCGCAACAGCCCCACAG |
| | YQVTVRIENMEDKAT | ACACCAUCCUGUUCAUCCGGGCCGCUCGCCGCGUGCAUUCAGGC |
| | LVLQVVDKPSP | ACUUACCAGGUGACGGUGCGCAUUGAGAACAUGGAGGACAAGG |
| | (SEQ ID NO: 53) | CCACGCUGGUGCUGCAGGUUGUUGACAAGCCAAGUCCU |
| | | (SEQ ID NO: 65) |
| Human | PPSEPTHLAVEDVSDT | CCCCCCAGCGAACCCACCCACCUGGCAGUAGAGGACGUCUCUGA |
| MYBPC3 | TVSLKWRPPERVGAG | CACCACGGUCUCCCUCAAGUGGCGGCCCCCAGAGCGCGUGGGA |
| C7 | GLDGYSVEYCPEGCSE | GCAGGAGGCCUGGAUGGCUACAGCGUGGAGUACUGCCCAGAGG |
| | WVAALQGLIEHTSILV | GCUGCUCAGAGUGGGUGGCUGCCCUGCAGGGGCUGACAGAGCA |
| | KDLPTGARLLFRVRAH | CACAUCGAUACUGGUGAAGGACCUGCCCACGGGGGCCCGGCUG |
| | NMAGPGAPVTTTEPV | CUUUUCCGAGUGCGGGCACACAAUAUGGCAGGGCCUGGAGCCC |
| | TVQEILQRPR | CUGUUACCACCACGGAGCCGGUGACAGUGCAGGAGAUCCUGCA |
| | (SEQ ID NO: 54) | ACGGCCACGG |
| | | (SEQ ID NO: 66) |
| Human | ILQRPRLQLPRHLRQTI | AUCCUGCAACGGCCACGGCUUCAGCUGCCCAGGCACCUGCGCCA |
| MYBPC3 | QKKVGEPVNLLIPFQG | GACCAUUCAGAAGAAGGUCGGGGAGCCUGUGAACCUUCUCAUC |
| C8 | KPRPQVTWTKEGQPL | CCUUUCCAGGGCAAGCCCCGGCCUCAGGUGACCUGGACCAAAG |
| | AGEEVSIRNSPTDTILFI | AGGGGCAGCCCCUGGCAGGCGAGGAGGUGAGCAUCCGCAACAG |
| | RAARRVHSGTYQVTV | CCCCACAGACACCAUCCUGUUCAUCCGGGCCGCUCGCCGCGUGC |
| | RIENMEDKATLVLQV | AUUCAGGCACUUACCAGGUGACGGUGCGCAUUGAGAACAUGGA |
| | VDKPSP | GGACAAGGCCACGCUGGUGCUGCAGGUUGUUGACAAGCCAAGU |
| | (SEQ ID NO: 55) | CCU (SEQ ID NO: 67) |
| Human | PPSEPTHLAVEDVSDT | CCCCCCAGCGAACCCACCCACCUGGCAGUAGAGGACGUCUCUGA |
| MYBPC3 | TVSLKWRPPERVGAG | CACCACGGUCUCCCUCAAGUGGCGGCCCCCAGAGCGCGUGGGA |
| C7-C10 | GLDGYSVEYCPEGCSE | GCAGGAGGCCUGGAUGGCUACAGCGUGGAGUACUGCCCAGAGG |
| | WVAALQGLIEHTSILV | GCUGCUCAGAGUGGGUGGCUGCCCUGCAGGGGCUGACAGAGCA |
| | KDLPTGARLLFRVRAH | CACAUCGAUACUGGUGAAGGACCUGCCCACGGGGGCCCGGCUG |
| | NMAGPGAPVTTTEPV | CUUUUCCGAGUGCGGGCACACAAUAUGGCAGGGCCUGGAGCCC |
| | TVQEILQRPRLQLPRH | CUGUUACCACCACGGAGCCGGUGACAGUGCAGGAGAUCCUGCA |
| | LRQTIQKKVGEPVNLL | ACGGCCACGGCUUCAGCUGCCCAGGCACCUGCGCCAGACCAUUC |
| | IPFQGKPRPQVTWTKE | AGAAGAAGGUCGGGGAGCCUGUGAACCUUCUCAUCCCUUUCCA |
| | GQPLAGEEVSIRNSPT | GGGCAAGCCCCGGCCUCAGGUGACCUGGACCAAAGAGGGGCAG |
| | DTILFIRAARRVHSGT | CCCCUGGCAGGCGAGGAGGUGAGCAUCCGCAACAGCCCCACAG |
| | YQVTVRIENMEDKAT | ACACCAUCCUGUUCAUCCGGGCCGCUCGCCGCGUGCAUUCAGGC |
| | LVLQVVDKPSPPQDLR | ACUUACCAGGUGACGGUGCGCAUUGAGAACAUGGAGGACAAGG |
| | VTDAWGLNVALEWK | CCACGCUGGUGCUGCAGGUUGUUGACAAGCCAAGUCCUCCCCA |
| | PPQDVGNTELWGYTV | GGAUCUCCGGGUGACUGACGCCUGGGGUCUUAAUGUGGCUCUG |
| | QKADKKTMEWFTVLE | GAGUGGAAGCCACCCCAGGAUGUCGGCAACACGGAGCUCUGGG |
| | HYRRTHCVVPELIIGN | GGUACACAGUGCAGAAAGCCGACAAGAAGACCAUGGAGUGGUU |
| | GYYFRVFSQNMVGFS | CACCGUCUUGGAGCAUUACCGCCGCACCCACUGCGUGGUGCCA |
| | DRAATTKEPVFIPRPGI | GAGCUCAUCAUUGGCAAUGGCUACUACUUCCGCGUCUUCAGCC |
| | TYEPPNYKALDFSEAP | AGAAUAUGGUUGGCUUUAGUGACAGAGCGGCCACCACCAAGGA |
| | SFTQPLVNRSVIAGYT | GCCCGUCUUUAUCCCCAGACCAGGCAUCACCUAUGAGCCACCCA |
| | AMLCCAVRGSPKPKIS | ACUAUAAGGCCCUGGACUUCUCCGAGGCCCCAAGCUUCACCCA |
| | WFKNGLDLGEDARFR | GCCCCUGGUGAACCGCUCGGUCAUCGCGGGCUACACUGCUAUG |
| | MFSKQGVLTLEIRKPC | CUCUGCUGUGCUGUCCGGGGUAGCCCCAAGCCCAAGAUUUCCU |
| | PFDGGIYVCRATNLQG | GGUUCAAGAAUGGCCUGGACCUGGGAGAAGACGCCCGCUUCCG |
| | EARCECRLEVRVPQ | CAUGUUCAGCAAGCAGGGAGUGUUGACUCUGGAGAUUAGAAAG |
| | (SEQ ID NO: 56) | CCCUGCCCCUUUGACGGGGGCAUCUAUGUCUGCAGGGCCACCA |
| | | ACUUACAGGGCGAGGCACGGUGUGAGUGCCGCCUGGAGGUGCG |
| | | AGUGCCUCAG |
| | | (SEQ ID NO: 68) |
| Human | VPDAPAAPKISNVGED | GUGCCAGACGCACCUGCGGCCCCCAAGAUCAGCAACGUGGGAG |
| MYBP C3 | SCTVQWEPPAYDGGQ | AGGACUCCUGCACAGUACAGUGGGAGCCGCCUGCCUACGAUGG |
| C6, C8-C10 | PILGYILERKKKKSYR | CGGGCAGCCCAUCCUGGGCUACAUCCUGGAGCGCAAGAAGAAG |
| | WMRLNFDLIQELSHEA | AAGAGCUACCGGUGGAUGCGGCUGAACUUCGACCUGAUUCAGG |
| | RRMIEGVVYEMRVYA | AGCUGAGUCAUGAAGCGCGGCGCAUGAUCGAGGGCGUGGUGUA |
| | VNAIGMSRPSPASQPF | CGAGAUGCGCGUCUACGCGGUCAACGCCAUCGGCAUGUCCAGG |
| | MPILQRPRLQLPRHLR | CCCAGCCCUGCCUCCCAGCCCUUCAUGCCUAUCCUGCAACGGCC |
| | QTIQKKVGEPVNLLIPF | ACGGCUUCAGCUGCCCAGGCACCUGCGCCAGACCAUUCAGAAG |
| | QGKPRPQVTWTKEGQ | AAGGUCGGGGAGCCUGUGAACCUUCUCAUCCCUUUCCAGGGCA |
| | PLAGEEVSIRNSPTDTI | AGCCCCGGCCUCAGGUGACCUGGACCAAAGAGGGGCAGCCCCU |
| | LFIRAARRVHSGTYQV | GGCAGGCGAGGAGGUGAGCAUCCGCAACAGCCCCACAGACACC |
| | TVRIENMEDKATLVLQ | AUCCUGUUCAUCCGGGCCGCUCGCCGCGUGCAUUCAGGCACUU |
| | VVDKPSPPQDLRVTDA | ACCAGGUGACGGUGCGCAUUGAGAACAUGGAGGACAAGGCCAC |
| | WGLNVALEWKPPQDV | GCUGGUGCUGCAGGUUGUUGACAAGCCAAGUCCUCCCCAGGAU |
| | GNTELWGYTVQKADK | CUCCGGGUGACUGACGCCUGGGGUCUUAAUGUGGCUCUGGAGU |
| | KTMEWFTVLEHYRRT | GGAAGCCACCCCAGGAUGUCGGCAACACGGAGCUCUGGGGGUA |
| | HCVVPELIIGNGYYFR | CACAGUGCAGAAAGCCGACAAGAAGACCAUGGAGUGGUUCACC |
| | VFSQNMVGFSDRAAT | GUCUUGGAGCAUUACCGCCGCACCCACUGCGUGGUGCCAGAGC |
| | TKEPVFIPRPGITYEPP | UCAUCAUUGGCAAUGGCUACUACUUCCGCGUCUUCAGCCAGAA |
| | NYKALDFSEAPSFTQP | UAUGGUUGGCUUUAGUGACAGAGCGGCCACCACCAAGGAGCCC |
| | LVNRSVIAGYTAMLC | GUCUUUAUCCCCAGACCAGGCAUCACCUAUGAGCCACCCAACU |
| | CAVRGSPKPKISWFKN | AUAAGGCCCUGGACUUCUCCGAGGCCCCAAGCUUCACCCAGCCC |
| | GLDLGEDARFRMFSK | CUGGUGAACCGCUCGGUCAUCGCGGGCUACACUGCUAUGCUCU |
| | QGVLTLEIRKPCPFDG | GCUGUGCUGUCCGGGGUAGCCCCAAGCCCAAGAUUUCCUGGUU |
| | GIYVCRATNLQGEARC | CAAGAAUGGCCUGGACCUGGGAGAAGACGCCCGCUUCCGCAUG |
| | ECRLEVRVPQ | UUCAGCAAGCAGGGAGUGUUGACUCUGGAGAUUAGAAAGCCCU |
| | (SEQ ID NO: 57) | GCCCCUUUGACGGGGGCAUCUAUGUCUGCAGGGCCACCAACUU |
| | | ACAGGGCGAGGCACGGUGUGAGUGCCGCCUGGAGGUGCGAGUG |
| | | CCUCAGUGA |
| | | (SEQ ID NO: 69) |
| Human | VPDAPAAPKISNVGED | GUGCCAGACGCACCUGCGGCCCCCAAGAUCAGCAACGUGGGAG |
| MYBPC3 | SCTVQWEPPAYDGGQ | AGGACUCCUGCACAGUACAGUGGGAGCCGCCUGCCUACGAUGG |
| C6-C7, | PILGYILERKKKKSYR | CGGGCAGCCCAUCCUGGGCUACAUCCUGGAGCGCAAGAAGAAG |
| C9-C10 | WMRLNFDLIQELSHEA | AAGAGCUACCGGUGGAUGCGGCUGAACUUCGACCUGAUUCAGG |
| | RRMIEGVVYEMRVYA | AGCUGAGUCAUGAAGCGCGGCGCAUGAUCGAGGGCGUGGUGUA |
| | VNAIGMSRPSPASQPF | CGAGAUGCGCGUCUACGCGGUCAACGCCAUCGGCAUGUCCAGG |
| | MPIGPPSEPTHLAVED | CCCAGCCCUGCCUCCCAGCCCUUCAUGCCUAUCGGUCCCCCCAG |
| | VSDTTVSLKWRPPERV | CGAACCCACCCACCUGGCAGUAGAGGACGUCUCUGACACCACG |
| | GAGGLDGYSVEYCPE | GUCUCCCUCAAGUGGCGGCCCCCAGAGCGCGUGGGAGCAGGAG |
| | GCSEWVAALQGLTEH | GCCUGGAUGGCUACAGCGUGGAGUACUGCCCAGAGGGCUGCUC |
| | TSILVKDLPTGARLLFR | AGAGUGGGUGGCUGCCCUGCAGGGGCUGACAGAGCACACAUCG |
| | VRAHNMAGPGAPVTT | AUACUGGUGAAGGACCUGCCCACGGGGGCCCGGCUGCUUUUCC |
| | TEPVTVQEILQRPRQV | GAGUGCGGGCACACAAUAUGGCAGGGCCUGGAGCCCCUGUUAC |
| | VDKPSPPQDLRVTDA | CACCACGGAGCCGGUGACAGUGCAGGAGAUCCUGCAACGGCCA |
| | WGLNVALEWKPPQDV | CGGCAGGUUGUUGACAAGCCAAGUCCUCCCCAGGAUCUCCGGG |
| | GNTELWGYTVQKADK | UGACUGACGCCUGGGGUCUUAAUGUGGCUCUGGAGUGGAAGCC |
| | KTMEWFTVLEHYRRT | ACCCCAGGAUGUCGGCAACACGGAGCUCUGGGGGUACACAGUG |
| | HCVVPELIIGNGYYFR | CAGAAAGCCGACAAGAAGACCAUGGAGUGGUUCACCGUCUUGG |
| | VFSQNMVGFSDRAAT | AGCAUUACCGCCGCACCCACUGCGUGGUGCCAGAGCUCAUCAU |
| | TKEPVFIPRPGITYEPP | UGGCAAUGGCUACUACUUCCGCGUCUUCAGCCAGAAUAUGGUU |
| | NYKALDFSEAPSFTQP | GGCUUUAGUGACAGAGCGGCCACCACCAAGGAGCCCGUCUUUA |
| | LVNRSVIAGYTAMLC | UCCCCAGACCAGGCAUCACCUAUGAGCCACCCAACUAUAAGGCC |
| | CAVRGSPKPKISWFKN | CUGGACUUCUCCGAGGCCCCAAGCUUCACCCAGCCCCUGGUGAA |
| | GLDLGEDARFRMFSK | CCGCUCGGUCAUCGCGGGCUACACUGCUAUGCUCUGCUGUGCU |
| | QGVLTLEIRKPCPFDG | GUCCGGGGUAGCCCCAAGCCCAAGAUUUCCUGGUUCAAGAAUG |
| | GIYVCRATNLQGEARC | GCCUGGACCUGGGAGAAGACGCCCGCUUCCGCAUGUUCAGCAA |
| | ECRLEVRVPQ | GCAGGGAGUGUUGACUCUGGAGAUUAGAAAGCCCUGCCCCUUU |
| | (SEQ ID NO: 58) | GACGGGGGCAUCUAUGUCUGCAGGGCCACCAACUUACAGGGCG |
| | | AGGCACGGUGUGAGUGCCGCCUGGAGGUGCGAGUGCCUCAG |
| | | (SEQ ID NO: 70) |
| Mouse | PPGEPTHLAVEDVSDT | CCCCCUGGCGAACCAACCCACUUGGCUGUGGAGGAUGUGUCAG |
| MYBPC3 | TVSLKWRPPERVGAG | ACACCACUGUCUCACUCAAGUGGCGGCCCCCAGAGCGCGUGGG |
| C7-C8 | GLDGYSVEYCQEGCS | GGCCGGUGGCCUGGACGGAUACAGCGUGGAGUACUGCCAGGAG |
| | EWTPALQGLIERTSM | GGAUGCUCCGAGUGGACACCUGCUCUGCAGGGGCUGACAGAGC |
| | LVKDLPTGARLLFRVR | GCACAUCGAUGCUGGUGAAGGACCUACCCACUGGGGCACGGCU |
| | AHNVAGPGGPIVTKEP | GCUGUUCCGAGUACGGGCACACAAUGUGGCAGGUCCUGGAGGC |
| | VTVQEILQRPRLQLPR | CCUAUCGUCACCAAGGAGCCUGUGACAGUGCAGGAGAUACUGC |
| | HLRQTIQKKVGEPVNL | AACGACCACGGCUCCAACUGCCCAGACACCUGCGCCAGACCAUC |
| | LIPFQGKPRPQVTWTK | CAGAAGAAAGUUGGGGAGCCUGUGAACCUCCUCAUCCCUUUCC |
| | EGQPLAGEEVSIRNSPT | AGGGCAAACCCCGGCCUCAGGUGACCUGGACCAAAGAGGGGCA |
| | DTILFIRAARRTHSGTY | GCCCCUGGCAGGUGAGGAGGUGAGCAUCCGGAACAGCCCCACA |
| | QVTVRIENMEDKATLI | GACACGAUCUUGUUCAUCCGAGCUGCCCGCCGCACCCACUCGGG |
| | LQIVDKPSP | CACCUACCAGGUGACAGUUCGCAUUGAGAACAUGGAGGACAAG |
| | (SEQ ID NO: 59) | GCAACGCUGAUCCUGCAGAUUGUGGACAAGCCAAGUCCU |
| | | (SEQ ID NO: 71) |
| Mouse | PPGEPTHLAVEDVSDT | CCCCCUGGCGAACCAACCCACUUGGCUGUGGAGGAUGUGUCAG |
| MYBPC3 | TVSLKWRPPERVGAG | ACACCACUGUCUCACUCAAGUGGCGGCCCCCAGAGCGCGUGGG |
| C7 | GLDGYSVEYCQEGCS | GGCCGGUGGCCUGGACGGAUACAGCGUGGAGUACUGCCAGGAG |
| | EWTPALQGLIERTSM | GGAUGCUCCGAGUGGACACCUGCUCUGCAGGGGCUGACAGAGC |
| | LVKDLPTGARLLFRVR | GCACAUCGAUGCUGGUGAAGGACCUACCCACUGGGGCACGGCU |
| | AHNVAGPGGPIVTKEP | GCUGUUCCGAGUACGGGCACACAAUGUGGCAGGUCCUGGAGGC |
| | VTVQEILQRPR | CCUAUCGUCACCAAGGAGCCUGUGACAGUGCAGGAGAUACUGC |
| | (SEQ ID NO: 60) | AACGACCACGG |
| | | (SEQ ID NO: 72) |
| Mouse | ILQRPRLQLPRHLRQTI | AUACUGCAACGACCACGGCUCCAACUGCCCAGACACCUGCGCCA |
| MYBPC3 | QKKVGEPVNLLIPFQG | GACCAUCCAGAAGAAAGUUGGGGAGCCUGUGAACCUCCUCAUC |
| C8 | KPRPQVTWTKEGQPL | CCUUUCCAGGGCAAACCCCGGCCUCAGGUGACCUGGACCAAAG |
| | AGEEVSIRNSPTDTILFI | AGGGGCAGCCCCUGGCAGGUGAGGAGGUGAGCAUCCGGAACAG |
| | RAARRTHSGTYQVTV | CCCCACAGACACGAUCUUGUUCAUCCGAGCUGCCCGCCGCACCC |
| | RIENMEDKATLILQIV | ACUCGGGCACCUACCAGGUGACAGUUCGCAUUGAGAACAUGGA |
| | DKPSP | GGACAAGGCAACGCUGAUCCUGCAGAUUGUGGACAAGCCAAGU |
| | (SEQ ID NO: 61) | CCU |
| | | (SEQ ID NO: 73) |
| Mouse | PPGEPTHLAVEDVSDT | CCCCCUGGCGAACCAACCCACUUGGCUGUGGAGGAUGUGUCAG |
| MYBPC3 | TVSLKWRPPERVGAG | ACACCACUGUCUCACUCAAGUGGCGGCCCCCAGAGCGCGUGGG |
| C7-C10 | GLDGYSVEYCQEGCS | GGCCGGUGGCCUGGACGGAUACAGCGUGGAGUACUGCCAGGAG |
| | EWTPALQGLIERTSM | GGAUGCUCCGAGUGGACACCUGCUCUGCAGGGGCUGACAGAGC |
| | LVKDLPTGARLLFRVR | GCACAUCGAUGCUGGUGAAGGACCUACCCACUGGGGCACGGCU |
| | AHNVAGPGGPIVTKEP | GCUGUUCCGAGUACGGGCACACAAUGUGGCAGGUCCUGGAGGC |
| | VTVQEILQRPRLQLPR | CCUAUCGUCACCAAGGAGCCUGUGACAGUGCAGGAGAUACUGC |
| | HLRQTIQKKVGEPVNL | AACGACCACGGCUCCAACUGCCCAGACACCUGCGCCAGACCAUC |
| | LIPFQGKPRPQVTWTK | CAGAAGAAAGUUGGGGAGCCUGUGAACCUCCUCAUCCCUUUCC |
| | EGQPLAGEEVSIRNSPT | AGGGCAAACCCCGGCCUCAGGUGACCUGGACCAAAGAGGGGCA |
| | DTILFIRAARRTHSGTY | GCCCCUGGCAGGUGAGGAGGUGAGCAUCCGGAACAGCCCCACA |
| | QVTVRIENMEDKATLI | GACACGAUCUUGUUCAUCCGAGCUGCCCGCCGCACCCACUCGGG |
| | LQIVDKPSPPQDIRIVE | CACCUACCAGGUGACAGUUCGCAUUGAGAACAUGGAGGACAAG |
| | TWGFNVALEWKPPQD | GCAACGCUGAUCCUGCAGAUUGUGGACAAGCCAAGUCCUCCCC |
| | DGNTEIWGYTVQKAD | AGGAUAUCCGGAUCGUUGAGACUUGGGGUUUCAAUGUGGCUCU |
| | KKTMEWFTVLEHYRR | GGAGUGGAAGCCACCCCAAGAUGAUGGCAAUACAGAGAUCUGG |
| | THCVVSELIIGNGYYF | GGUUAUACUGUACAGAAAGCUGACAAGAAGACCAUGGAGUGGU |
| | RVFSHNMVGSSDKAA | UCACGGUUUUGGAACACUACCGACGCACUCACUGUGUGGUAUC |
| | ATKEPVFIPRPGITYEP | AGAGCUUAUCAUUGGCAAUGGCUACUACUUCCGGGUCUUCAGC |
| | PKYKALDFSEAPSFTQ | CAUAACAUGGUGGGUUCCAGUGACAAAGCUGCCGCCACCAAGG |
| | PLANRSIIAGYNAILCC | AGCCAGUCUUUAUUCCAAGACCAGGCAUCACAUAUGAGCCACC |
| | AVRGSPKPKISWFKNG | CAAAUACAAGGCCCUGGACUUCUCUGAGGCCCCAAGCUUCACC |
| | LDLGEDARFRMFCKQ | CAGCCCUUGGCAAAUCGCUCCAUCAUUGCAGGCUAUAAUGCCA |
| | GVLTLEIRKPCPYDGG | UCCUCUGCUGUGCUGUCCGAGGUAGUCCUAAGCCCAAGAUUUC |
| | VYVCRATNLQGEAQC | CUGGUUCAAGAAUGGCCUGGAUCUGGGAGAAGAUGCUCGCUUC |
| | ECRLEVRVPQ | CGCAUGUUCUGCAAGCAGGGAGUAUUGACCCUGGAGAUCAGGA |
| | (SEQ ID NO: 62) | AACCCUGCCCCUAUGAUGGUGGUGUCUAUGUCUGCAGGGCCAC |
| | | CAACUUGCAGGGCGAGGCACAGUGUGAGUGCCGCCUGGAGGUG |
| | | CGAGUUCCUCAG |
| | | (SEQ ID NO: 74) |
| Mouse | VPDAPAAPKISNVGED | GUCCCAGAUGCUCCUGCGGCCCCUAAGAUCAGCAACGUGGGCG |
| MYBPC3 | SCTVQWEPPAYDGGQ | AGGACUCCUGCACUGUGCAGUGGGAACCGCCUGCCUAUGAUGG |
| C6, C8-C10 | PVLGYILERKKKKSYR | CGGGCAGCCGGUCCUGGGAUACAUCCUGGAGCGCAAGAAGAAA |
| | WMRLNFDLLRELSHE | AAGAGCUACAGGUGGAUGAGGCUCAACUUUGAUCUGCUGCGGG |
| | ARRMIEGVAYEMRVY | AGCUGAGCCACGAGGCGAGGCGCAUGAUCGAGGGUGUAGCCUA |
| | AVNAVGMSRPSPASQ | UGAGAUGCGAGUCUACGCAGUCAAUGCCGUGGGAAUGUCCAGG |
| | PFMPILQRPRLQLPRHL | CCCAGCCCUGCCUCUCAGCCCUUCAUGCCUAUACUGCAACGACC |
| | RQTIQKKVGEPVNLLI | ACGGCUCCAACUGCCCAGACACCUGCGCCAGACCAUCCAGAAGA |
| | PFQGKPRPQVTWTKE | AAGUUGGGGAGCCUGUGAACCUCCUCAUCCCUUUCCAGGGCAA |
| | GQPLAGEEVSIRNSPT | ACCCCGGCCUCAGGUGACCUGGACCAAAGAGGGGCAGCCCCUG |
| | DTILFIRAARRTHSGTY | GCAGGUGAGGAGGUGAGCAUCCGGAACAGCCCCACAGACACGA |
| | QVTVRIENMEDKATLI | UCUUGUUCAUCCGAGCUGCCCGCCGCACCCACUCGGGCACCUAC |
| | LQIVDKPSPPQDIRIVE | CAGGUGACAGUUCGCAUUGAGAACAUGGAGGACAAGGCAACGC |
| | TWGFNVALEWKPPQD | UGAUCCUGCAGAUUGUGGACAAGCCAAGUCCUCCCCAGGAUAU |
| | DGNTEIWGYTVQKAD | CCGGAUCGUUGAGACUUGGGGUUUCAAUGUGGCUCUGGAGUGG |
| | KKTMEWFTVLEHYRR | AAGCCACCCCAAGAUGAUGGCAAUACAGAGAUCUGGGGUUAUA |
| | THCVVSELIIGNGYYF | CUGUACAGAAAGCUGACAAGAAGACCAUGGAGUGGUUCACGGU |
| | RVFSHNMVGSSDKAA | UUUGGAACACUACCGACGCACUCACUGUGUGGUAUCAGAGCUU |
| | ATKEPVFIPRPGITYEP | AUCAUUGGCAAUGGCUACUACUUCCGGGUCUUCAGCCAUAACA |
| | PKYKALDFSEAPSFTQ | UGGUGGGUUCCAGUGACAAAGCUGCCGCCACCAAGGAGCCAGU |
| | PLANRSIIAGYNAILCC | CUUUAUUCCAAGACCAGGCAUCACAUAUGAGCCACCCAAAUAC |
| | AVRGSPKPKISWFKNG | AAGGCCCUGGACUUCUCUGAGGCCCCAAGCUUCACCCAGCCCUU |
| | LDLGEDARFRMFCKQ | GGCAAAUCGCUCCAUCAUUGCAGGCUAUAAUGCCAUCCUCUGC |
| | GVLTLEIRKPCPYDGG | UGUGCUGUCCGAGGUAGUCCUAAGCCCAAGAUUUCCUGGUUCA |
| | VYVCRATNLQGEAQC | AGAAUGGCCUGGAUCUGGGAGAAGAUGCUCGCUUCCGCAUGUU |
| | ECRLEVRVPQ | CUGCAAGCAGGGAGUAUUGACCCUGGAGAUCAGGAAACCCUGC |
| | (SEQ ID NO: 63) | CCCUAUGAUGGUGGUGUCUAUGUCUGCAGGGCCACCAACUUGC |
| | | AGGGCGAGGCACAGUGUGAGUGCCGCCUGGAGGUGCGAGUUCC |
| | | UCAG |
| | | (SEQ ID NO: 75) |
| Mouse | VPDAPAAPKISNVGED | GUCCCAGAUGCUCCUGCGGCCCCUAAGAUCAGCAACGUGGGCG |
| MYBPC3 | SCTVQWEPPAYDGGQ | AGGACUCCUGCACUGUGCAGUGGGAACCGCCUGCCUAUGAUGG |
| C6-C7, C9- | PVLGYILERKKKKSYR | CGGGCAGCCGGUCCUGGGAUACAUCCUGGAGCGCAAGAAGAAA |
| C10 | WMRLNFDLLRELSHE | AAGAGCUACAGGUGGAUGAGGCUCAACUUUGAUCUGCUGCGGG |
| | ARRMIEGVAYEMRVY | AGCUGAGCCACGAGGCGAGGCGCAUGAUCGAGGGUGUAGCCUA |
| | AVNAVGMSRPSPASQ | UGAGAUGCGAGUCUACGCAGUCAAUGCCGUGGGAAUGUCCAGG |
| | PFMPIGPPGEPTHLAVE | CCCAGCCCUGCCUCUCAGCCCUUCAUGCCUAUUGGGCCCCCUGG |
| | DVSDTTVSLKWRPPER | CGAACCAACCCACUUGGCUGUGGAGGAUGUGUCAGACACCACU |
| | VGAGGLDGYSVEYCQ | GUCUCACUCAAGUGGCGGCCCCCAGAGCGCGUGGGGGCCGGUG |
| | EGCSEWTPALQGLTER | GCCUGGACGGAUACAGCGUGGAGUACUGCCAGGAGGGAUGCUC |
| | TSMLVKDLPTGARLLF | CGAGUGGACACCUGCUCUGCAGGGGCUGACAGAGCGCACAUCG |
| | RVRAHNVAGPGGPIVT | AUGCUGGUGAAGGACCUACCCACUGGGGCACGGCUGCUGUUCC |
| | KEPVTVQEILQRPRQIV | GAGUACGGGCACACAAUGUGGCAGGUCCUGGAGGCCCUAUCGU |
| | DKPSPPQDIRIVETWGF | CACCAAGGAGCCUGUGACAGUGCAGGAGAUACUGCAACGACCA |
| | NVALEWKPPQDDGNT | CGGCAGAUUGUGGACAAGCCAAGUCCUCCCCAGGAUAUCCGGA |
| | EIWGYTVQKADKKTM | UCGUUGAGACUUGGGGUUUCAAUGUGGCUCUGGAGUGGAAGCC |
| | EWFTVLEHYRRTHCV | ACCCCAAGAUGAUGGCAAUACAGAGAUCUGGGGUUAUACUGUA |
| | VSELIIGNGYYFRVFSH | CAGAAAGCUGACAAGAAGACCAUGGAGUGGUUCACGGUUUUGG |
| | NMVGSSDKAAATKEP | AACACUACCGACGCACUCACUGUGUGGUAUCAGAGCUUAUCAU |
| | VFIPRPGITYEPPKYKA | UGGCAAUGGCUACUACUUCCGGGUCUUCAGCCAUAACAUGGUG |
| | LDFSEAPSFTQPLANRS | GGUUCCAGUGACAAAGCUGCCGCCACCAAGGAGCCAGUCUUUA |
| | IIAGYNAILCCAVRGSP | UUCCAAGACCAGGCAUCACAUAUGAGCCACCCAAAUACAAGGC |
| | KPKISWFKNGLDLGED | CCUGGACUUCUCUGAGGCCCCAAGCUUCACCCAGCCCUUGGCAA |
| | ARFRMFCKQGVLTLEI | AUCGCUCCAUCAUUGCAGGCUAUAAUGCCAUCCUCUGCUGUGC |
| | RKPCPYDGGVYVCRA | UGUCCGAGGUAGUCCUAAGCCCAAGAUUUCCUGGUUCAAGAAU |
| | TNLQGEAQCECRLEV | GGCCUGGAUCUGGGAGAAGAUGCUCGCUUCCGCAUGUUCUGCA |
| | RVPQ | AGCAGGGAGUAUUGACCCUGGAGAUCAGGAAACCCUGCCCCUA |
| | (SEQ ID NO: 64) | UGAUGGUGGUGUCUAUGUCUGCAGGGCCACCAACUUGCAGGGC |
| | | GAGGCACAGUGUGAGUGCCGCCUGGAGGUGCGAGUUCCUCAG |
| | | (SEQ ID NO: 76) |

In some embodiments, the polypeptide used in the methods described herein comprises an amino acid sequence that is at least 80% (e.g., at least 80%, at least 85%, at least 90%, at least 95%, or at least 99%) identical to any one of SEQ ID NOs: 1-16 or 53-64. In some embodiments, the polypeptide used in the methods described herein comprises an amino acid sequence that is 80%, 85%, 90%, 95%, or 99% identical to any one of SEQ ID NOs: 1-16 or 53-64. In some embodiments, the polypeptide used in the methods described herein comprises the amino acid sequence of SEQ ID NOs: 1-16 or 53-64.

In some embodiments, the nucleic acid used in the methods described herein comprises a nucleotide sequence encoding the polypeptide (e.g., a polypeptide comprising a C-terminal domain of MYBPC3 described herein). In some embodiments, the nucleic acid used in the methods described herein comprises a nucleotide sequence that is at least 80% (e.g., at least 80%, at least 85%, at least 90%, at least 95%, or at least 99%) identical to any one of SEQ ID NOs: 17-32 or 65-76. In some embodiments, the nucleic acid used in the methods described herein comprises a nucleotide sequence that is 80%, 85%, 90%, 95%, or 99% identical to any one of SEQ ID NOs: 17-32 or 65-76. In some embodiments, the nucleic acid used in the methods described herein comprises the nucleotide sequence of SEQ ID NOs: 17-32 or 65-76.

As used herein, “nucleic acids” may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) or chimeras or combinations thereof. The nucleic acids molecules of the present disclosure may be DNA or RNA. The skilled artisan will appreciate that, except where otherwise noted, nucleic acid sequences set forth in the present disclosure will recite “T”s in a representative DNA sequence but where the sequence represents RNA, the “T”s would be substituted for “U”s.

In some embodiments, the nucleotide sequence encoding the polypeptide (e.g., a polypeptide comprising a C-terminal domain of MYBPC3 described herein) is operably linked to a promoter.

A “promoter” is a control region of a nucleic acid at which initiation and rate of transcription of the remainder of a nucleic acid are controlled. A promoter may also contain sub-regions at which regulatory proteins and molecules, such as transcription factors, bind. Promoters of the present disclosure may be constitutive, inducible, activatable, repressible, tissue-specific or any combination thereof. A promoter drives expression or drives transcription of the nucleic acid that it regulates. A promoter is considered to be “operably linked” when it is in a correct functional location and orientation in relation to the nucleic acid it regulates to control (“drive”) transcriptional initiation and/or expression of that nucleic acid. In some embodiments, the promoter is a constitutive promoter. In some embodiments, the promoter is an inducible promoter (also referred to as regulatable promoter).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an enhanced chicken β-actin promoter. In some embodiments, a promoter is a U6 promoter. In some embodiments, the promoter used in present disclosure is a CAG promoter (e.g., containing a CMV enhancer, a promoter and the first exon and the first intron from the chicken beta-actin gene, and a splice acceptor of the rabbit beta-globin gene, as described in Okabe et al., FEB S Lett. 1997 May 5; 407(3):313-9; and Alexopoulou et al., BMC Cell Biology 9: 2, 2008, incorporated herein by reference).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In some embodiments, inducible promoters that include a repressor with the operon can be used. In one embodiment, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used (Yao et al., Human Gene Therapy; Gossen et al., Natl. Acad. Sci. USA, 89:5547-5551 (1992); Shockett et al., Proc. Natl. Acad. Sci. USA, 92:6522-6526 (1995)).

In some embodiments, the native promoter for MYBPC3 used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the promoter is a tissue-specific promoter containing regulatory sequences that impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, the nucleic acid used in the method described herein is a messenger RNA (mRNA). A “messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. In some preferred embodiments, an mRNA is translated in vivo. The skilled artisan will appreciate that, except where otherwise noted, polynucleotide sequences set forth in the instant application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U.” One of ordinary skill in the art would understand how to identify an mRNA sequence based on the corresponding DNA sequence.

The basic components of an mRNA molecule typically include at least one coding region, a 5′ untranslated region (UTR), a 3′ UTR, a 5′ cap and a poly-A tail. Polynucleotides of the present disclosure may function as mRNA but can be distinguished from wild-type mRNA in their functional and/or structural design features which serve to overcome existing problems of effective polypeptide expression using nucleic-acid based therapeutics.

In some embodiments, the mRNA described herein comprises one or more chemical modifications (e.g., comprises one or more modified nucleotides). The terms “chemical modification” and “chemically modified” refer to modification with respect to adenosine (A), guanosine (G), uridine (U), thymidine (T) or cytidine (C) ribonucleosides or deoxyribnucleosides in at least one of their position, pattern, percent or population. Generally, these terms do not refer to the ribonucleotide modifications in naturally occurring 5′-terminal mRNA cap moieties.

The mRNAs described herein, some embodiments, comprise various (more than one) different modifications. In some embodiments, a particular region of a mRNA contains one, two or more (optionally different) nucleoside or nucleotide modifications. In some embodiments, a modified mRNA, introduced to a cell or organism, exhibits reduced degradation in the cell or organism, respectively, relative to an unmodified mRNA. In some embodiments, a modified mRNA introduced into a cell or organism, may exhibit reduced immunogenicity in the cell or organism, respectively (e.g., a reduced innate response).

Modifications of polynucleotides include, without limitation, those described herein. Modified mRNAs of the present disclosure may comprise modifications that are naturally-occurring, non-naturally-occurring or the polynucleotide may comprise a combination of naturally-occurring and non-naturally-occurring modifications. The mRNAs may include any useful modification, for example, of a sugar, a nucleobase, or an internucleoside linkage (e.g., to a linking phosphate, to a phosphodiester linkage or to the phosphodiester backbone).

The mRNAs described herein, in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

In some embodiments, the modified mRNA comprises one or more modified nucleosides and nucleotides. A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

In some embodiments, modified nucleobases in the modified mRNA described herein are selected from the group consisting of pseudouridine (ψ), N1-methylpseudouridine (m1ψ), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In some embodiments, the nucleic acid used in the methods described herein is a vector (e.g., a cloning vector or an expression vector). The vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the polypeptides described herein. In some embodiments, the expression of the polypeptides described herein is regulated by a constitutive, an inducible or a tissue-specific promoter.

A variety of host-expression vector systems may be utilized in accordance with the present disclosure. Such host-expression systems represent vehicles by which the nucleotide sequences described herein may be produced and subsequently purified, but also represent cells which may, when transformed or transfected with the appropriate nucleotide sequences, express the polypeptide (e.g., a polypeptide comprising a C-terminal domain of MYBPC3 described herein) in situ. These include, but are not limited to, microorganisms such as bacteria (e.g., E. coli and B. subtilis) transformed with recombinant bacteriophage DNA, plasmid DNA or cosmid DNA expression vectors containing the nucleotide sequence encoding the polypeptide (e.g., a polypeptide comprising a C-terminal domain of MYBPC3 described herein); yeast (e.g., Saccharomyces pichia) transformed with recombinant yeast expression vectors containing nucleotide sequence encoding the polypeptide (e.g., a polypeptide comprising a C-terminal domain of MYBPC3 described herein); insect cell systems infected with recombinant virus expression vectors (e.g., baclovirus) containing the nucleotide sequence encoding the polypeptide (e.g., a polypeptide comprising a C-terminal domain of MYBPC3 described herein); plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing nucleotide sequence encoding the polypeptide (e.g., a polypeptide comprising a C-terminal domain of MYBPC3 described herein); or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In some embodiments, the vector of the present disclosure is a viral vector. In some embodiments, the viral vector is suitable for mammalian expression of the polypeptide (e.g., a polypeptide comprising a C-terminal domain of MYBPC3 described herein). Suitable viral vectors include lentiviral vectors, retroviral vectors, or a recombinant adeno-associated virus (rAAV) vectors.

A “lentiviral vector” refers to a vector derived from a lentivirus genome (e.g., HIV). Lentiviral vectors have been commonly used in gene therapy, e.g., to insert beneficial genes into a host cell or organism, or to delete or modify a gene in a host cell or organism. Lentiviral vectors are efficient vehicles for gene transfer in mammalian cells due to their capacity to stably express a gene of interest in non-dividing and dividing cells.

A “retroviral vector” refers to a vector derived from a retrovirus genome. A retroviral vector consists of proviral sequences that can accommodate the gene of interest, to allow incorporation of both into the target cells. The vector also contains viral and cellular gene promoters, such as the CMV promoter, to enhance expression of the gene of interest in the target cells. Retroviral vectors have also been commonly used in gene therapy.

A “recombinant adeno-associated virus (rAAV) vector” is typically composed of, at a minimum, a transgene and its regulatory sequences (e.g., a promoter), and 5′ and 3′ AAV inverted terminal repeats (ITRs). The transgene may comprise, as disclosed elsewhere herein, a nucleotide sequence encoding, for example, a polypeptide comprising a C-terminal domain of MYBPC3, as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the rAAV vectors described herein comprises two ITRs flanking (one ITR on each end of the sequence being flanked) the nucleotide sequence encoding the polypeptide (e.g., a polypeptide comprising a C-terminal domain of MYBPC3 described herein). In some embodiments, the nucleotide sequence encoding the polypeptide (e.g., a polypeptide comprising a C-terminal domain of MYBPC3 described herein) is operably linked to a promoter and the rAAV vectors described herein comprises two ITRs flanking (one ITR on each end of the sequence being flanked) the nucleotide sequence encoding the polypeptide (e.g., a polypeptide comprising a C-terminal domain of MYBPC3 described herein) and the promoter.

In some embodiments, the ITRs are of a serotype selected from AAV1, AAV2, AAV2i8, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the rAAV vector comprises ITRs of serotype AAV2. In some embodiments, the ITR used in the rAAV vector described herein comprises the nucleotide sequence of:

(SEQ ID NO: 33)
CTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGA
GGGAGTGGCCAACTCCATCACTAGGGGTTCCTTGTAGTTAATGATTAAC
CCGCCATGCTACTTATCTACG.

In some embodiments, the rAAV vector of the present disclosure is a self-complementary AAV vector (scAAV). A “self-complementary AAV vector” (scAAV) refers to a vector containing a double-stranded vector genome generated by the absence of a terminal resolution site (TR) from one of the ITRs of the AAV (e.g., as described in McCarthy (2008) Molecular Therapy 16(10):1648-1656, incorporated herein by reference). The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle. The instant invention is based, in part, on the recognition that DNA fragments encoding RNA hairpin structures (e.g. shRNA, miRNA, and AmiRNA) can serve a function similar to a mutant inverted terminal repeat (mTR) during viral genome replication, generating self-complementary AAV vector genomes. In some embodiments, the ITR used in the scAAV vector described herein comprises the nucleotide sequence of:

(SEQ ID NO: 34)
CTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGA
GGGAGTGG.

Further provided herein, in some aspects, are recombinant adeno-associated virus (rAAV) comprising a capsid protein and any one of the nucleic acid molecules described herein. In some embodiments, a “capsid protein” refers to structural proteins encoded by the CAP gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host.

In some embodiments, an AAV capsid protein is of an AAV serotype selected from the group consisting of AAV1, AAV2, AAV2i8, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125. In some embodiments, an AAV capsid protein is of a serotype derived from a non-human primate, for example scAAV.rh8, AAV.rh39, or AAV.rh43 serotype. In some embodiments, an AAV capsid protein is of an AAV9 serotype. In some embodiments, an AAV capsid protein is of an AAV2i8 serotype. Non-limiting examples of the amino acid sequences of capsid proteins are provided as SEQ ID NOs: 35-52.

SEQ ID NO 35: AAV-CAPSID 1
MAADGYLPDWLEDNLSEGIREWWDLKPGAPKPKANQQKQDDGRGLVLPGYKYLGP
FNGLDKGEPVNAADAAALEHDKAYDQQLKAGDNPYLRYNHADAEFQERLQEDTSFG
GNLGRAVFQAKKRVLEPLGLVEEGAKTAPGKKRPVEQSPQEPDSSSGIGKTGQQPAK
KRLNFGQTGDSESVPDPQPLGEPPATPAAVGPTTMASGGGAPMADNNEGADGVGNA
SGNWHCDSTWLGDRVITTSTRTWALPTYNNHLYKQISSASTGASNDNHYFGYSTPWG
YFDFNRFHCHFSPRDWQRLINNNWGFRPKRLNFKLFNIQVKEVTTNDGVTTIANNLTS
TVQVFSDSEYQLPYVLGSAHQGCLPPFPADVFMIPQYGYLTLNNGSQAVGRSSFYCLE
YFPSQMLRTGNNFTFSYTFEEVPFHSSYAHSQSLDRLMNPLIDQYLYYLNRTQNQSGS
AQNKDLLFSRGSPAGMSVQPKNWLPGPCYRQQRVSKTKTDNNNSNFTWTGASKYNL
NGRESIINPGTAMASHKDDEDKFFPMSGVMIFGKESAGASNTALDNVMITDEEEIKAT
NPVATERFGTVAVNFQSSSTDPATGDVHAMGALPGMVWQDRDVYLQGPIWAKIPHT
DGHFHPSPLMGGFGLKNPPPQILIKNTPVPANPPAEFSATKFASFITQYSTGQVSVEIEW
ELQKENSKRWNPEVQYTSNYAKSANVDFTVDNNGLYTEPRPIGTRYLTRPL
SEQ ID NO 36: AAV-CAPSID 2
MAADGYLPDWLEDTLSEGIRQWWKLKPGPPPPKPAERHKDDSRGLVLPGYKYLGPF
NGLDKGEPVNEADAAALEHDKAYDRQLDSGDNPYLKYNHADAEFQERLKEDTSFGG
NLGRAVFQAKKRVLEPLGLVEEPVKTAPGKKRPVEHSPVEPDSSSGTGKAGQQPARK
RLNFGQTGDADSVPDPQPLGQPPAAPSGLGTNTMATGSGAPMADNNEGADGVGNSS
GNWHCDSTWMGDRVITTSTRTWALPTYNNHLYKQISSQSGASNDNHYFGYSTPWGY
FDFNRFHCHFSPRDWQRLINNNWGFRPKRLNFKLFNIQVKEVTQNDGTTTIANNLTST
VQVFTDSEYQLPYVLGSAHQGCLPPFPADVFMVPQYGYLTLNNGSQAVGRSSFYCLE
YFPSQMLRTGNNFTFSYTFEDVPFHSSYAHSQSLDRLMNPLIDQYLYYLSRTNTPSGTT
TQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPV
ATEQYGSVSTNLQRGNRQAATADVNTQGVLPGMVWQDRDVYLQGPIWAKIPHTDG
HFHPSPLMGGFGLKEIPPPQILIKNTPVPANPSTTFSAAKFASFITQYSTGQVSVEIEWEL
QKENSKRWNPEIQYTSNYNKSVNVDFTVDTNGVYSEPRPIGTRYLTRNL
SEQ ID NO 37: AAV-CAPSID 3B
MAADGYLPDWLEDNLSEGIREWWALKPGVPQPKANQQHQDNRRGLVLPGYKYLGP
GNGLDKGEPVNEADAAALEHDKAYDQQLKAGDNPYLKYNHADAEFQERLQEDTSF
GGNLGRAVFQAKKRILEPLGLVEEAAKTAPGKKRPVDQSPQEPDSSSGVGKSGKQPA
RKRLNFGQTGDSESVPDPQPLGEPPAAPTSLGSNTMASGGGAPMADNNEGADGVGNS
SGNWHCDSQWLGDRVITTSTRTWALPTYNNHLYKQISSQSGASNDNHYFGYSTPWG
YFDFNRFHCHFSPRDWQRLINNNWGFRPKKLSFKLFNIQVKEVTQNDGTTTIANNLTS
TVQVFTDSEYQLPYVLGSAHQGCLPPFPADVFMVPQYGYLTLNNGSQAVGRSSFYCL
EYFPSQMLRTGNNFQFSYTFEDVPFHSSYAHSQSLDRLMNPLIDQYLYYLNRTQGTTS
GTTNQSRLLFSQAGPQSMSLQARNWLPGPCYRQQRLSKTANDNNNSNFPWTAASKY
HLNGRDSLVNPGPAMASHKDDEEKFFPMHGNLIFGKEGTTASNAELDNVMITDEEEIR
TTNPVATEQYGTVANNLQSSNTAPTTRTVNDQGALPGMVWQDRDVYLQGPIWAKIP
HTDGHFHPSPLMGGFGLKEIPPPQIMIKNTPVPANPPTTFSPAKFASFITQYSTGQVSVEI
EWELQKENSKRWNPEIQYTSNYNKSVNVDFTVDTNGVYSEPRPIGTRYLTRNL
SEQ ID NO 38: AAV-CAPSID 4
MTDGYLPDWLEDNLSEGVREWWALQPGAPKPKANQQHQDNARGLVLPGYKYLGPG
NGLDKGEPVNAADAAALEHDKAYDQQLKAGDNPYLKYNHADAEFQQRLQGDTSFG
GNLGRAVFQAKKRVLEPLGLVEQAGETAPGKKRPLIESPQQPDSSTGIGKKGKQPAKK
KLVFEDETGAGDGPPEGSTSGAMSDDSEMRAAAGGAAVEGGQGADGVGNASGDWH
CDSTWSEGHVTTTSTRTWVLPTYNNHLYKRLGESLQSNTYNGFSTPWGYFDFNRFHC
HFSPRDWQRLINNNWGMRPKAMRVKIFNIQVKEVTTSNGETTVANNLTSTVQIFADSS
YELPYVMDAGQEGSLPPFPNDVFMVPQYGYCGLVTGNTSQQQTDRNAFYCLEYFPSQ
MLRTGNNFEITYSFEKVPFHSMYAHSQSLDRLMNPLIDQYLWGLQSTTTGTTLNAGTA
TTNFTKLRPTNFSNFKKNWLPGPSIKQQGFSKTANQNYKIPATGSDSLIKYETHSTLDG
RWSALTPGPPMATAGPADSKFSNSQLIFAGPKQNGNTATVPGTLIFTSEEELAATNATD
TDMWGNLPGGDQSNSNLPTVDRLTALGAVPGMVWQNRDIYYQGPIWAKIPHTDGHF
HPSPLIGGFGLKHPPPQIFIKNTPVPANPATTFSSTPVNSFITQYSTGQVSVQIDWEIQKE
RSKRWNPEVQFTSNYGQQNSLLWAPDAAGKYTEPRAIGTRYLTHHL
SEQ ID NO 39: AAV-CAPSID 5
MSFVDHPPDWLEEVGEGLREFLGLEAGPPKPKPNQQHQDQARGLVLPGYNYLGPGN
GLDRGEPVNRADEVAREHDISYNEQLEAGDNPYLKYNHADAEFQEKLADDTSFGGNL
GKAVFQAKKRVLEPFGLVEEGAKTAPTGKRIDDHFPKRKKARTEEDSKPSTSSDAEAG
PSGSQQLQIPAQPASSLGADTMSAGGGGPLGDNNQGADGVGNASGDWHCDSTWMG
DRVVTKSTRTWVLPSYNNHQYREIKSGSVDGSNANAYFGYSTPWGYFDFNRFHSHW
SPRDWQRLINNYWGFRPRSLRVKIFNIQVKEVTVQDSTTTIANNLTSTVQVFTDDDYQ
LPYVVGNGTEGCLPAFPPQVFTLPQYGYATLNRDNTENPTERSSFFCLEYFPSKMLRT
GNNFEFTYNFEEVPFHSSFAPSQNLFKLANPLVDQYLYRFVSTNNTGGVQFNKNLAGR
YANTYKNWFPGPMGRTQGWNLGSGVNRASVSAFATTNRMELEGASYQVPPQPNGM
TNNLQGSNTYALENTMIFNSQPANPGTTATYLEGNIVILITSESETQPVNRVAYNVGGQ
MATNNQSSTTAPATGTYNLQEIVPGSVWMERDVYLQGPIWAKIPETGAHFHPSPAMG
GFGLKHPPPMMLIKNTPVPGNITSFSDVPVSSFITQYSTGQVTVEMEWELKKENSKRW
NPEIQYTNNYNDPQFVDFAPDSTGEYRTTRPIGTRYLTRPL
SEQ ID NO 40: AAV-CAPSID 6
MAADGYLPDWLEDNLSEGIREWWDLKPGAPKPKANQQKQDDGRGLVLPGYKYLGP
FNGLDKGEPVNAADAAALEHDKAYDQQLKAGDNPYLRYNHADAEFQERLQEDTSFG
GNLGRAVFQAKKRVLEPFGLVEEGAKTAPGKKRPVEQSPQEPDSSSGIGKTGQQPAK
KRLNFGQTGDSESVPDPQPLGEPPATPAAVGPTTMASGGGAPMADNNEGADGVGNA
SGNWHCDSTWLGDRVITTSTRTWALPTYNNHLYKQISSASTGASNDNHYFGYSTPWG
YFDFNRFHCHFSPRDWQRLINNNWGFRPKRLNFKLFNIQVKEVTTNDGVTTIANNLTS
TVQVFSDSEYQLPYVLGSAHQGCLPPFPADVFMIPQYGYLTLNNGSQAVGRSSFYCLE
YFPSQMLRTGNNFTFSYTFEDVPFHSSYAHSQSLDRLMNPLIDQYLYYLNRTQNQSGS
AQNKDLLFSRGSPAGMSVQPKNWLPGPCYRQQRVSKTKTDNNNSNFTWTGASKYNL
NGRESIINPGTAMASHKDDKDKFFPMSGVMIFGKESAGASNTALDNVMITDEEEIKAT
NPVATERFGTVAVNLQSSSTDPATGDVHVMGALPGMVWQDRDVYLQGPIWAKIPHT
DGHFHPSPLMGGFGLKEIPPPQILIKNTPVPANPPAEFSATKFASFITQYSTGQVSVEIEW
ELQKENSKRWNPEVQYTSNYAKSANVDFTVDNNGLYTEPRPIGTRYLTRPL
SEQ ID NO 41: AAV-CAPSID 6.2
MAADGYLPDWLEDNLSEGIREWWDLKPGAPKPKANQQKQDDGRGLVLPGYKYLGP
FNGLDKGEPVNAADAAALEHDKAYDQQLKAGDNPYLRYNHADAEFQERLQEDTSFG
GNLGRAVFQAKKRVLEPLGLVEEGAKTAPGKKRPVEQSPQEPDSSSGIGKTGQQPAK
KRLNFGQTGDSESVPDPQPLGEPPATPAAVGPTTMASGGGAPMADNNEGADGVGNA
SGNWHCDSTWLGDRVITTSTRTWALPTYNNHLYKQISSASTGASNDNHYFGYSTPWG
YFDFNRFHCHFSPRDWQRLINNNWGFRPKRLNFKLFNIQVKEVTTNDGVTTIANNLTS
TVQVFSDSEYQLPYVLGSAHQGCLPPFPADVFMIPQYGYLTLNNGSQAVGRSSFYCLE
YFPSQMLRTGNNFTFSYTFEDVPFHSSYAHSQSLDRLMNPLIDQYLYYLNRTQNQSGS
AQNKDLLFSRGSPAGMSVQPKNWLPGPCYRQQRVSKTKTDNNNSNFTWTGASKYNL
NGRESIINPGTAMASHKDDKDKFFPMSGVMIFGKESAGASNTALDNVMITDEEEIKAT
NPVATERFGTVAVNLQSSSTDPATGDVHVMGALPGMVWQDRDVYLQGPIWAKIPHT
DGHFHPSPLMGGFGLKEIPPPQILIKNTPVPANPPAEFSATKFASFITQYSTGQVSVEIEW
ELQKENSKRWNPEVQYTSNYAKSANVDFTVDNNGLYTEPRPIGTRYLTRPL
SEQ ID NO 42: AAV-CAPSID 7
MAADGYLPDWLEDNLSEGIREWWDLKPGAPKPKANQQKQDNGRGLVLPGYKYLGP
FNGLDKGEPVNAADAAALEHDKAYDQQLKAGDNPYLRYNHADAEFQERLQEDTSFG
GNLGRAVFQAKKRVLEPLGLVEEGAKTAPAKKRPVEPSPQRSPDSSTGIGKKGQQPAR
KRLNFGQTGDSESVPDPQPLGEPPAAPSSVGSGTVAAGGGAPMADNNEGADGVGNAS
GNWHCDSTWLGDRVITTSTRTWALPTYNNHLYKQISSETAGSTNDNTYFGYSTPWGY
FDFNRFHCHFSPRDWQRLINNNWGFRPKKLRFKLFNIQVKEVTTNDGVTTIANNLTSTI
QVFSDSEYQLPYVLGSAHQGCLPPFPADVFMIPQYGYLTLNNGSQSVGRSSFYCLEYF
PSQMLRTGNNFEFSYSFEDVPFHSSYAHSQSLDRLMNPLIDQYLYYLARTQSNPGGTA
GNRELQFYQGGPSTMAEQAKNWLPGPCFRQQRVSKTLDQNNNSNFAWTGATKYHL
NGRNSLVNPGVAMATHKDDEDRFFPSSGVLIFGKTGATNKTTLENVLMTNEEEIRPTN
PVATEEYGIVSSNLQAANTAAQTQVVNNQGALPGMVWQNRDVYLQGPIWAKIPHTD
GNFHPSPLMGGFGLKHPPPQILIKNTPVPANPPEVFTPAKFASFITQYSTGQVSVEIEWE
LQKENSKRWNPEIQYTSNFEKQTGVDFAVDSQGVYSEPRPIGTRYLTRNL
SEQ ID NO 43: AAV-CAPSID 8
MAADGYLPDWLEDNLSEGIREWWALKPGAPKPKANQQKQDDGRGLVLPGYKYLGP
FNGLDKGEPVNAADAAALEHDKAYDQQLQAGDNPYLRYNHADAEFQERLQEDTSFG
GNLGRAVFQAKKRVLEPLGLVEEGAKTAPGKKRPVEPSPQRSPDSSTGIGKKGQQPAR
KRLNFGQTGDSESVPDPQPLGEPPAAPSGVGPNTMAAGGGAPMADNNEGADGVGSSS
GNWHCDSTWLGDRVITTSTRTWALPTYNNHLYKQISNGTSGGATNDNTYFGYSTPW
GYFDFNRFHCHFSPRDWQRLINNNWGFRPKRLSFKLFNIQVKEVTQNEGTKTIANNLT
STIQVFTDSEYQLPYVLGSAHQGCLPPFPADVFMIPQYGYLTLNNGSQAVGRSSFYCLE
YFPSQMLRTGNNFQFTYTFEDVPFHSSYAHSQSLDRLMNPLIDQYLYYLSRTQTTGGT
ANTQTLGFSQGGPNTMANQAKNWLPGPCYRQQRVSTTTGQNNNSNFAWTAGTKYH
LNGRNSLANPGIAMATHKDDEERFFPSNGILIFGKQNAARDNADYSDVMLTSEEEIKT
TNPVATEEYGIVADNLQQQNTAPQIGTVNSQGALPGMVWQNRDVYLQGPIWAKIPHT
DGNFHPSPLMGGFGLKHPPPQILIKNTPVPADPPTTFNQSKLNSFITQYSTGQVSVEIEW
ELQKENSKRWNPEIQYTSNYYKSTSVDFAVNTEGVYSEPRPIGTRYLTRNL
SEQ ID NO 44: AAV-CAPSID 9
MAADGYLPDWLEDNLSEGIREWWALKPGAPQPKANQQHQDNARGLVLPGYKYLGP
GNGLDKGEPVNAADAAALEHDKAYDQQLKAGDNPYLKYNHADAEFQERLKEDTSF
GGNLGRAVFQAKKRLLEPLGLVEEAAKTAPGKKRPVEQSPQEPDSSAGIGKSGAQPA
KKRLNFGQTGDTESVPDPQPIGEPPAAPSGVGSLTMASGGGAPVADNNEGADGVGSS
SGNWHCDSQWLGDRVITTSTRTWALPTYNNHLYKQISNSTSGGSSNDNAYFGYSTPW
GYFDFNRFHCHFSPRDWQRLINNNWGFRPKRLNFKLFNIQVKEVTDNNGVKTIANNL
TSTVQVFTDSDYQLPYVLGSAHEGCLPPFPADVFMIPQYGYLTLNDGSQAVGRSSFYC
LEYFPSQMLRTGNNFQFSYEFENVPFHSSYAHSQSLDRLMNPLIDQYLYYLSKTINGSG
QNQQTLKFSVAGPSNMAVQGRNYIPGPSYRQQRVSTTVTQNNNSEFAWPGASSWAL
NGRNSLMNPGPAMASHKEGEDRFFPLSGSLIFGKQGTGRDNVDADKVMITNEEEIKTT
NPVATESYGQVATNHQSAQAQAQTGWVQNQGILPGMVWQDRDVYLQGPIWAKIPH
TDGNFHPSPLMGGFGMKHPPPQILIKNTPVPADPPTAFNKDKLNSFITQYSTGQVSVEIE
WELQKENSKRWNPEIQYTSNYYKSNNVEFAVNTEGVYSEPRPIGTRYLTRNL
SEQ ID NO 45: AAV-CAPSID rh8
MAADGYLPDWLEDNLSEGIREWWDLKPGAPKPKANQQKQDDGRGLVLPGYKYLGP
FNGLDKGEPVNAADAAALEHDKAYDQQLKAGDNPYLRYNHADAEFQERLQEDTSFG
GNLGRAVFQAKKRVLEPLGLVEEGAKTAPGKKRPVEQSPQEPDSSSGIGKTGQQPAK
KRLNFGQTGDSESVPDPQPLGEPPAAPSGLGPNTMASGGGAPMADNNEGADGVGNSS
GNWHCDSTWLGDRVITTSTRTWALPTYNNHLYKQISNGTSGGSTNDNTYFGYSTPWG
YFDFNRFHCHFSPRDWQRLINNNWGFRPKRLNFKLFNIQVKEVTTNEGTKTIANNLTS
TVQVFTDSEYQLPYVLGSAHQGCLPPFPADVFMVPQYGYLTLNNGSQALGRSSFYCL
EYFPSQMLRTGNNFQFSYTFEDVPFHSSYAHSQSLDRLMNPLIDQYLYYLVRTQTTGT
GGTQTLAFSQAGPSSMANQARNWVPGPCYRQQRVSTTTNQNNNSNFAWTGAAKFKL
NGRDSLMNPGVAMASHKDDDDRFFPSSGVLIFGKQGAGNDGVDYSQVLITDEEEIKA
TNPVATEEYGAVAINNQAANTQAQTGLVHNQGVIPGMVWQNRDVYLQGPIWAKIPH
TDGNFHPSPLMGGFGLKHPPPQILIKNTPVPADPPLTFNQAKLNSFITQYSTGQVSVEIE
WELQKENSKRWNPEIQYTSNYYKSTNVDFAVNTEGVYSEPRPIGTRYLTRNL
SEQ ID NO 46: AAV-CAPSID rh10
MAADGYLPDWLEDNLSEGIREWWDLKPGAPKPKANQQKQDDGRGLVLPGYKYLGP
FNGLDKGEPVNAADAAALEHDKAYDQQLKAGDNPYLRYNHADAEFQERLQEDTSFG
GNLGRAVFQAKKRVLEPLGLVEEGAKTAPGKKRPVEPSPQRSPDSSTGIGKKGQQPAK
KRLNFGQTGDSESVPDPQPIGEPPAGPSGLGSGTMAAGGGAPMADNNEGADGVGSSS
GNWHCDSTWLGDRVITTSTRTWALPTYNNHLYKQISNGTSGGSTNDNTYFGYSTPWG
YFDFNRFHCHFSPRDWQRLINNNWGFRPKRLNFKLFNIQVKEVTQNEGTKTIANNLTS
TIQVFTDSEYQLPYVLGSAHQGCLPPFPADVFMIPQYGYLTLNNGSQAVGRSSFYCLE
YFPSQMLRTGNNFEFSYQFEDVPFHSSYAHSQSLDRLMNPLIDQYLYYLSRTQSTGGT
AGTQQLLFSQAGPNNMSAQAKNWLPGPCYRQQRVSTTLSQNNNSNFAWTGATKYHL
NGRDSLVNPGVAMATHKDDEERFFPSSGVLMFGKQGAGKDNVDYSSVMLTSEEEIKT
TNPVATEQYGVVADNLQQQNAAPIVGAVNSQGALPGMVWQNRDVYLQGPIWAKIPH
TDGNFHPSPLMGGFGLKHPPPQILIKNTPVPADPPTTFSQAKLASFITQYSTGQVSVEIE
WELQKENSKRWNPEIQYTSNYYKSTNVDFAVNTDGTYSEPRPIGTRYLTRNL
SEQ ID NO 47: AAV-CAPSID rh39
MAADGYLPDWLEDNLSEGIREWWALKPGAPKPKANQQKQDDGRGLVLPGYKYLGP
FNGLDKGEPVNAADAAALEHDKAYDQQLKAGDNPYLRYNHADAEFQERLQEDTSFG
GNLGRAVFQAKKRVLEPLGLVEEAAKTAPGKKRPVEPSPQRSPDSSTGIGKKGQQPAK
KRLNFGQTGDSESVPDPQPIGEPPAGPSGLGSGTMAAGGGAPMADNNEGADGVGSSS
GNWHCDSTWLGDRVITTSTRTWALPTYNNHLYKQISNGTSGGSTNDNTYFGYSTPWG
YFDFNRFHCHFSPRDWQRLINNNWGFRPKRLSFKLFNIQVKEVTQNEGTKTIANNLTS
TIQVFTDSEYQLPYVLGSAHQGCLPPFPADVFMIPQYGYLTLNNGSQAVGRSSFYCLE
YFPSQMLRTGNNFEFSYTFEDVPFHSSYAHSQSLDRLMNPLIDQYLYYLSRTQSTGGT
QGTQQLLFSQAGPANMSAQAKNWLPGPCYRQQRVSTTLSQNNNSNFAWTGATKYHL
NGRDSLVNPGVAMATHKDDEERFFPSSGVLMFGKQGAGRDNVDYSSVMLTSEEEIKT
TNPVATEQYGVVADNLQQTNTGPIVGNVNSQGALPGMVWQNRDVYLQGPIWAKIPH
TDGNFHPSPLMGGFGLKHPPPQILIKNTPVPADPPTTFSQAKLASFITQYSTGQVSVEIE
WELQKENSKRWNPEIQYTSNYYKSTNVDFAVNTEGTYSEPRPIGTRYLTRNL
SEQ ID NO 48: AAV-CAPSID rh43
MAADGYLPDWLEDNLSEGIREWWDLKPGAPKPKANQQKQDDGRGLVLPGYKYLGP
FNGLDKGEPVNAADAAALEHDKAYDQQLEAGDNPYLRYNHADAEFQERLQEDTSFG
GNLGRAVFQAKKRVLEPLGLVEEGAKTAPGKKRPVEQSPQEPDSSSGIGKKGQQPAR
KRLNFGQTGDSESVPDPQPLGEPPAAPSGVGPNTMAAGGGAPMADNNEGADGVGSSS
GNWHCDSTWLGDRVITTSTRTWALPTYNNHLYKQISNGTSGGATNDNTYFGYSTPW
GYFDFNRFHCHFSPRDWQRLINNNWGFRPKRLSFKLFNIQVKEVTQNEGTKTIANNLT
STIQVFTDSEYQLPYVLGSAHQGCLPPFPADVFMIPQYGYLTLNNGSQAVGRSSFYCLE
YFPSQMLRTGNNFQFTYTFEDVPFHSSYAHSQSLDRLMNPLIDQYLYYLSRTQTTGGT
ANTQTLGFSQGGPNTMANQAKNWLPGPCYRQQRVSTTTGQNNNSNFAWTAGTKYH
LNGRNSLANPGIAMATHKDDEERFFPSNGILIFGKQNAARDNADYSDVMLTSEEEIKT
TNPVATEEYGIVADNLQQQNTAPQIGTVNSQGALPGMVWQNRDVYLQGPIWAKIPHT
DGNFHPSPLMGGFGLKHPPPQILIKNTPVPADPPTTFNQSKLNSFITQYSTGQVSVEIEW
ELQKENSKRWNPEIQYTSNYYKSTSVDFAVNTEGVYSEPRPIGTRYLTRNL
SEQ ID NO 49: AAV-CAPSID 2/2-66
MAADGYLPDWLEDTLSEGIRQWWKLKPGPPPPKPAERHQDDSRGLVLPGYKYLGPF
NGLDKGEPVNEADAAALEHDKAYDRQLDSGDNPYLKYNHADAEFQERLKEDTSFGG
NLGRAVFQAKKRVLEPLGLVEEPVKTAPGKKRPVEHSPAEPDSSSGTGKAGQQPARK
RLNFGQTGDADSVPDPQPLGQPPAAPSGLGTNTMATGSGAPMADNNEGADGVGNSS
GNWHCDSTWMGDRVITTSTRTWALPTYNNHLYKQISSQSGASNDNHYFGYSTPWGY
FDFNRFHCHFSPRDWQRLINNNWGFRPKRLNFKLFNIQVKEVTQNDGTTTIANNLTST
VQVFTDSEYQLPYVLGSAHQGCLPPFPADVFMVPQYGYLTLNNGSQAVGRSSFYCLE
YFPSQMLRTGNNFTFSYTFEDVPFHSSYAHSQSLDRLMNPLIDQYLYYLSKTNAPSGTT
TMSRLQFSQAGASDIRDQSRNWLPGPCYRQQRVSKTAADNNNSDYSWTGATKYHLN
GRDSLVNPGPAMASHKDDEEKYFPQSGVLIFGKQDSGKTNVDIEKVMITDEEEIRTTN
PVATEQYGSVSTNLQSGNTQAATTDVNTQGVLPGMVWQDRDVYLQGPIWAKIPHTD
GHFHPSPLMGGFGLKHPPPQILIKNTPVPANPSTTFSAAKFASFITQYSTGQVSVEIEWE
LQKENSKRWNPEIQYTSNYNKSVNVDFTVDTNGVYSEPRPIGTRYLTRNL
SEQ ID NO 50: AAV-CAPSID 2/2-84
MAADGYLPDWLEDTLSEGIRQWWKLKPGPPPPKPAERHQDDSRGLVLPGYKYLGPF
NGLDKGEPVNEADAAALEHDKAYDRQLDSGDNPYLKYNHADAEFQERLKEDTSFGG
NLGRAVFQAKKRVLEPLGLVEEPVKTAPGKKRPVEHSPAEPDSSSGTGKAGQQPARK
RLNFGQTGDADSVPDPQPLGQPPAAPSGLGTNTMATGSGAPMADNNEGADGVGNSS
GNWHCDSTWMGDRVITTSTRTWALPTYNNHLYKQISSQSGASNDNHYFGYSTPWGY
FDFNRFHCHFSPRDWQRLINNNWGFRPKRLNFKLFNIQVKEVTQNDGTTTIANNLTST
VQVFTDSEYQLPYVLGSAHQGCLPPFPADVFMVPQYGYLTLNNGSQAVGRSSFYCLE
YFPSQMLRTGNNFTFSYTFEDVPFHSSYAHSQSLDRLMNPLIDQYLYYLSKTNAPSGTT
TMSRLQFSQAGASDIRDQSRNWLPGPCYRQQRVSKTAADNNNSDYSWTGATKYHLN
GRDSLVNPGPAMASHKDDEEKYFPQSGVLIFGKQDSGKTNVDIEKVMITDEEEIRTTN
PVATEQYGSVSTNLQSGNTQAATTDVNTQGVLPGMVWQDRDVYLQGPIWAKIPHTD
GHFHPSPLMGGFGLKHPPPQILIKNTPVPANPSTTFSAAKLASFITQYSTGQVSVEIEWE
LQKENSKRWNPEIQYTSNYNKSVNVDFTVDTNGVYSEPRPIGTRYLTRNL
SEQ ID NO 51: AAV-CAPSID 2/2-125
MAADGYLPDWLEDTLSEGIRQWWKLKPGPPPPKPAERHKDDSRGLVLPGYKYLGPF
NGLDKGEPVNEADAAALEHDKAYDRQLDSGDNPYLKYNHADAEFQERLKEDTSFGG
NLARAVFQAKKRVLEPLGLVEEPVKTAPGKKRPVEHSPAEPDSSSGTGKSGQQPARK
RLNFGQTGDADSVPDPQPLGQPPAAPSGLGTNTMASGSGAPMADNNEGADGVGNSS
GNWHCDSTWMGDRVITTSTRTWALPTYNNHLYKQISSQSGASNDNHYFGYSTPWGY
FDFNRFHCHFSPRDWQRLINNNWGFRPKRLNFKLFNIQVKEVTQNDGTTTIANNLTST
VQVFTDSEYQLPYVLGSAHQGCLPPFPADVFMVPQYGYLTLNNGSQTVGRSSFYCLE
YFPSQMLRTGNNFTFSYTFEDVPFHSSYAHSQSLDRLMNPLIDQYLYYLSRTNTPSGTT
TQSRLRFSQAGASDIRDQSRNWLPGPCYRQQRVSKTAADNNNSDYSWTGATKYHLN
GRDSLVNPGTAMASHKDDEEKYFPQSGVLIFGKQDSGKTNVDIERVMITDEEEIRTTN
PVATEQYGSVSTNLQSGNTQAATSDVNTQGVLPGMVWQDRDVYLQGPIWAKIPHTD
GHFHPSPLMGGFGLKHPPPQILIKNTPVPANPSTTFSAAKFASFITQYSTGQVSVEIEWE
LQKENSKRWNPEIQYTSNYNKSVNVDFTVDTNGVYSEPRPIGTRYLTRNL
SEQ ID NO 52: AAV-CAPSID 2i8 (substitution of RGNRQA (amino acids 585-590)
of AAV2-CAPSID with QQNTAP)
MAADGYLPDWLEDTLSEGIRQWWKLKPGPPPPKPAERHKDDSRGLVLPGYKYLGPF
NGLDKGEPVNEADAAALEHDKAYDRQLDSGDNPYLKYNHADAEFQERLKEDTSFGG
NLGRAVFQAKKRVLEPLGLVEEPVKTAPGKKRPVEHSPVEPDSSSGTGKAGQQPARK
RLNFGQTGDADSVPDPQPLGQPPAAPSGLGTNTMATGSGAPMADNNEGADGVGNSS
GNWHCDSTWMGDRVITTSTRTWALPTYNNHLYKQISSQSGASNDNHYFGYSTPWGY
FDFNRFHCHFSPRDWQRLINNNWGFRPKRLNFKLFNIQVKEVTQNDGTTTIANNLTST
VQVFTDSEYQLPYVLGSAHQGCLPPFPADVFMVPQYGYLTLNNGSQAVGRSSFYCLE
YFPSQMLRTGNNFTFSYTFEDVPFHSSYAHSQSLDRLMNPLIDQYLYYLSRTNTPSGTT
TQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPV
ATEQYGSVSTNLQQQNTAPATADVNTQGVLPGMVWQDRDVYLQGPIWAKIPHTDGH
FHPSPLMGGFGLKHPPPQILIKNTPVPANPSTTFSAAKFASFITQYSTGQVSVEIEWELQ
KENSKRWNPEIQYTSNYNKSVNVDFTVDTNGVYSEPRPIGTRYLTRNL
AAV-cTnT-HA-hC7C8-P2A-GFP
SEQ ID NO: 77
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCTTGTAGTTAATGATTAACCCGCCATGCTACTTATCTAC
CAGGGTAATGGGGATCCTCTAGAACTATAGCTAGAATTCGCCCTTACGGGCCCCCC
CTCGAGGTCGGGATAAAAGCAGTCTGGGCTTTCACATGACAGCATCTGGGGCTGC
GGCAGAGGGTCGGGTCCGAAGCGCTGCCTTATCAGCGTCCCCAGCCCTGGGAGGT
GACAGCTGGCTGGCTTGTGTCAGCCCCTCGGGCACTCACGTATCTCCGTCCGACGG
GTTTAAAATAGCAAAACTCTGAGGCCACACAATAGCTTGGGCTTATATGGGCTCCT
GTGGGGGAAGGGGGAGCACGGAGGGGGCCGGGGCCGCTGCTGCCAAAATAGCAG
CTCACAAGTGTTGCATTCCTCTCTGGGCGCCGGGCACATTCCTGCTGGCTCTGCCC
GCCCCGGGGTGGGCGCCGGGGGGACCTTAAAGCCTCTGCCCCCCAAGGAGCCCTT
CCCAGACAGCCGCCGGCACCCACCGCTCCGTGGGACGATCCCCGAAGCTCTAGAG
CTTTATTGCGGTAGTTTATCACAGTTAAATTGCTAACGCAGTCAGTGCTTCTGACA
CAACAGTCTCGAACTTAAGCTGCAGAAGTTGGTCGTGAGGCACTGGGCAGGTAAG
TATCAAGGTTACAAGACAGGTTTAAGGAGACCAATAGAAACTGGGCTTGTCGAGA
CAGAGAAGACTCTTGCGTTTCTGATAGGCACCTATTGGTCTTACTGACATCCACTT
TGCCTTTCTCTCCACAGGTGTCCACTCCCAGTTCAATTACAGCTCTTAAGGCTAGA
GTACTTAATACGACTCACTATAGGCTAGCCTCGAGAAGcggccgcactactccgcggactactact
agtATGGCCGTTTACCCATACGATGTTCCTGACTATGCGGGCTATCCCTATGACGTC
CCGGACTATGCAGGATCCTATCCATATGACGTTCCAGATTACGCTaccggtCCCCCCA
GCGAACCCACCCACCTGGCAGTAGAGGACGTCTCTGACACCACGGTCTCCCTCAA
GTGGCGGCCCCCAGAGCGCGTGGGAGCAGGAGGCCTGGATGGCTACAGCGTGGA
GTACTGCCCAGAGGGCTGCTCAGAGTGGGTGGCTGCCCTGCAGGGGCTGACAGAG
CACACATCGATACTGGTGAAGGACCTGCCCACGGGGGCCCGGCTGCTTTTCCGAG
TGCGGGCACACAATATGGCAGGGCCTGGAGCCCCTGTTACCACCACGGAGCCGGT
GACAGTGCAGGAGATCCTGCAACGGCCACGGCTTCAGCTGCCCAGGCACCTGCGC
CAGACCATTCAGAAGAAGGTCGGGGAGCCTGTGAACCTTCTCATCCCTTTCCAGG
GCAAGCCCCGGCCTCAGGTGACCTGGACCAAAGAGGGGCAGCCCCTGGCAGGCG
AGGAGGTGAGCATCCGCAACAGCCCCACAGACACCATCCTGTTCATCCGGGCCGC
TCGCCGCGTGCATTCAGGCACTTACCAGGTGACGGTGCGCATTGAGAACATGGAG
GACAAGGCCACGCTGGTGCTGCAGGTTGTTGACAAGCCAAGTCCTaagettGGAcaattg
GGAgagctcGGATCCGGAGCCACGAACTTCTCTCTGTTAAAGCAAGCAGGAGACGTG
GAAGAAAACCCCGGTCCTGCCATGGTGAGCAAGGGCGAGGAGCTGTTCACCGGGG
TGGTGCCCATCCTGGTCGAGCTGGACGGCGACGTAAACGGCCACAAGTTCAGCGT
GTCCGGCGAGGGCGAGGGCGATGCCACCTACGGCAAGCTGACCCTGAAGTTCATC
TGCACCACCGGCAAGCTGCCCGTGCCCTGGCCCACCCTCGTGACCACCCTGACCTA
CGGCGTGCAGTGCTTCAGCCGCTACCCCGACCACATGAAGCAGCACGACTTCTTCA
AGTCCGCCATGCCCGAAGGCTACGTCCAGGAGCGCACCATCTTCTTCAAGGACGA
CGGCAACTACAAGACCCGCGCCGAGGTGAAGTTCGAGGGCGACACCCTGGTGAAC
CGCATCGAGCTGAAGGGCATCGACTTCAAGGAGGACGGCAACATCCTGGGGCACA
AGCTGGAGTACAACTACAACAGCCACAACGTCTATATCATGGCCGACAAGCAGAA
GAACGGCATCAAGGTGAACTTCAAGATCCGCCACAACATCGAGGACGGCAGCGTG
CAGCTCGCCGACCACTACCAGCAGAACACCCCCATCGGCGACGGCCCCGTGCTGC
TGCCCGACAACCACTACCTGAGCACCCAGTCCGCCCTGAGCAAAGACCCCAACGA
GAAGCGCGATCACATGGTCCTGCTGGAGTTCGTGACCGCCGCCGGGATCACTCTC
GGCATGGACGAGCTGTACAAGTAATAAGCTCGCGTGGTACCTCTAGAGTCGACCC
GGGCGGCCTCGAGGACGGGGTGAACTACGCCTGAGGATCCGATCTTTTTCCCTCTG
CCAAAAATTATGGGGACATCATGAAGCCCCTTGAGCATCTGACTTCTGGCTAATAA
AGGAAATTTATTTTCATTGCAATAGTGTGTTGGAATTTTTTGTGTCTCTCACTCGGA
AGCAATTCGTTGATCTGAATTTCGACCACCCATAATACCCATTACCCTGGTAGATA
AGTAGCATGGCGGGTTAATCATTAACTACAAGGAACCCCTAGTGATGGAGTTGGC
CACTCCCTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGCCTTAA
TTAACCTAATTCACTGGCCGTCGTTTTACAACGTCGTGACTGGGAAAACCCTGGCG
TTACCCAACTTAATCGCCTTGCAGCACATCCCCCTTTCGCCAGCTGGCGTAATAGC
GAAGAGGCCCGCACCGATCGCCCTTCCCAACAGTTGCGCAGCCTGAATGGCGAAT
GGGACGCGCCCTGTAGCGGCGCATTAAGCGCGGCGGGTGTGGTGGTTACGCGCAG
CGTGACCGCTACACTTGCCAGCGCCCTAGCGCCCGCTCCTTTCGCTTTCTTCCCTTC
CTTTCTCGCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATTAGGGTG
ATGGTTCACGTAGTGGGCCATCGCCCTGATAGACGGTTTTTCGCCCTTTGACGTTG
GAGTCCACGTTCTTTAATAGTGGACTCTTGTTCCAAACTGGAACAACACTCAACCC
TATCTCGGTCTATTCTTTTGATTTATAAGGGATTTTGCCGATTTCGGCCTATTGGTT
AAAAAATGAGCTGATTTAACAAAAATTTAACGCGAATTTTAACAAAATATTAACG
CTTACAATTTAGGTGGCACTTTTCGGGGAAATGTGCGCGGAACCCCTATTTGTTTA
TTTTTCTAAATACATTCAAATATGTATCCGCTCATGAGACAATAACCCTGATAAAT
GCTTCAATAATATTGAAAAAGGAAGAGTATGAGTATTCAACATTTCCGTGTCGCCC
TTATTCCCTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATCGAACT
GGATCTCAACAGCGGTAAGATCCTTGAGAGTTTTCGCCCCGAAGAACGTTTTCCAA
TGATGAGCACTTTTAAAGTTCTGCTATGTGGCGCGGTATTATCCCGTATTGACGCC
GGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTATG
CAGTGCTGCCATAACCATGAGTGATAACACTGCGGCCAACTTACTTCTGACAACG
ATCGGAGGACCGAAGGAGCTAACCGCTTTTTTGCACAACATGGGGGATCATGTAA
CTCGCCTTGATCGTTGGGAACCGGAGCTGAATGAAGCCATACCAAACGACGAGCG
TGACACCACGATGCCTGTAGCAATGGCAACAACGTTGCGCAAACTATTAACTGGC
GAACTACTTACTCTAGCTTCCCGGCAACAATTAATAGACTGGATGGAGGCGGATA
AAGTTGCAGGACCACTTCTGCGCTCGGCCCTTCCGGCTGGCTGGTTTATTGCTGAT
AAATCTGGAGCCGGTGAGCGTGGGTCTCGCGGTATCATTGCAGCACTGGGGCCAG
ATGGTAAGCCCTCCCGTATCGTAGTTATCTACACGACGGGGAGTCAGGCAACTAT
GGATGAACGAAATAGACAGATCGCTGAGATAGGTGCCTCACTGATTAAGCATTGG
TAACTGTCAGACCAAGTTTACTCATATATACTTTAGATTGATTTAAAACTTCATTTT
TAATTTAAAAGGATCTAGGTGAAGATCCTTTTTGATAATCTCATGACCAAAATCCC
TTAACGTGAGTTTTCGTTCCACTGAGCGTCAGACCCCGTAGAAAAGATCAAAGGA
TCTTCTTGAGATCCTTTTTTTCTGCGCGTAATCTGCTGCTTGCAAACAAAAAAACCA
CCGCTACCAGCGGTGGTTTGTTTGCCGGATCAAGAGCTACCAACTCTTTTTCCGAA
GGTAACTGGCTTCAGCAGAGCGCAGATACCAAATACTGTTCTTCTAGTGTAGCCGT
AGTTAGGCCACCACTTCAAGAACTCTGTAGCACCGCCTACATACCTCGCTCTGCTA
ATCCTGTTACCAGTGGCTGCTGCCAGTGGCGATAAGTCGTGTCTTACCGGGTTGGA
CTCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAGC
GTGAGCTATGAGAAAGCGCCACGCTTCCCGAAGGGAGAAAGGCGGACAGGTATC
CGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGAT
TTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTA
TCCCCTGATTCTGTGGATAACCGTATTACCGCCTTTGAGTGAGCTGATACCGCTCG
CCGCAGCCGAACGACCGAGCGCAGCGAGTCAGTGAGCGAGGAAGCGGAAGAGCG
CCCAATACGCAAACCGCCTCTCCCCGCGCGTTGGCCGATTCATTAATGCAGCTGGC
ACGACAGGTTTCCCGACTGGAAAGCGGGCAGTGAGCGCAACGCAATTAATGTGAG
TTAGCTCACTCATTAGGCACCCCAGGCTTTACACTTTATGCTTCCGGCTCGTATGTT
GTGTGGAATTGTGAGCGGATAACAATTTCACACAGGAAACAGCTATGACCATGAT
TACGCCAGATTTAATTAAGGCCTTAATTAGG
AAV-cTnT-HA-mC7C8-P2A-GFP
SEQ ID NO: 78
CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGCCAA
CTCCATCACTAGGGGTTCCTTGTAGTTAATGATTAACCCGCCATGCTACTTATCTAC
CAGGGTAATGGGGATCCTCTAGAACTATAGCTAGAATTCGCCCTTACGGGCCCCCC
CTCGAGGTCGGGATAAAAGCAGTCTGGGCTTTCACATGACAGCATCTGGGGCTGC
GGCAGAGGGTCGGGTCCGAAGCGCTGCCTTATCAGCGTCCCCAGCCCTGGGAGGT
GACAGCTGGCTGGCTTGTGTCAGCCCCTCGGGCACTCACGTATCTCCGTCCGACGG
GTTTAAAATAGCAAAACTCTGAGGCCACACAATAGCTTGGGCTTATATGGGCTCCT
GTGGGGGAAGGGGGAGCACGGAGGGGGCCGGGGCCGCTGCTGCCAAAATAGCAG
CTCACAAGTGTTGCATTCCTCTCTGGGCGCCGGGCACATTCCTGCTGGCTCTGCCC
GCCCCGGGGTGGGCGCCGGGGGGACCTTAAAGCCTCTGCCCCCCAAGGAGCCCTT
CCCAGACAGCCGCCGGCACCCACCGCTCCGTGGGACGATCCCCGAAGCTCTAGAG
CTTTATTGCGGTAGTTTATCACAGTTAAATTGCTAACGCAGTCAGTGCTTCTGACA
CAACAGTCTCGAACTTAAGCTGCAGAAGTTGGTCGTGAGGCACTGGGCAGGTAAG
TATCAAGGTTACAAGACAGGTTTAAGGAGACCAATAGAAACTGGGCTTGTCGAGA
CAGAGAAGACTCTTGCGTTTCTGATAGGCACCTATTGGTCTTACTGACATCCACTT
TGCCTTTCTCTCCACAGGTGTCCACTCCCAGTTCAATTACAGCTCTTAAGGCTAGA
GTACTTAATACGACTCACTATAGGCTAGCCTCGAGAAGcggccgcactactccgcggactactact
agtATGGCCGTTTACCCATACGATGTTCCTGACTATGCGGGCTATCCCTATGACGTC
CCGGACTATGCAGGATCCTATCCATATGACGTTCCAGATTACGCTaccggtTTCATGC
CTATTGGGCCCCCTGGCGAACCAACCCACTTGGCTGTGGAGGATGTGTCAGACAC
CACTGTCTCACTCAAGTGGCGGCCCCCAGAGCGCGTGGGGGCCGGTGGCCTGGAC
GGATACAGCGTGGAGTACTGCCAGGAGGGATGCTCCGAGTGGACACCTGCTCTGC
AGGGGCTGACAGAGCGCACATCGATGCTGGTGAAGGACCTACCCACTGGGGCACG
GCTGCTGTTCCGAGTACGGGCACACAATGTGGCAGGTCCTGGAGGCCCTATCGTC
ACCAAGGAGCCTGTGACAGTGCAGGAGATACTGCAACGACCACGGCTCCAACTGC
CCAGACACCTGCGCCAGACCATCCAGAAGAAAGTTGGGGAGCCTGTGAACCTCCT
CATCCCTTTCCAGGGCAAACCCCGGCCTCAGGTGACCTGGACCAAAGAGGGGCAG
CCCCTGGCAGGTGAGGAGGTGAGCATCCGGAACAGCCCCACAGACACGATCTTGT
TCATCCGAGCTGCCCGCCGCACCCACTCGGGCACCTACCAGGTGACAGTTCGCATT
GAGAACATGGAGGACAAGGCAACGaagcttGGAcaattgGGAgagctcGGATCCGGAGCCA
CGAACTTCTCTCTGTTAAAGCAAGCAGGAGACGTGGAAGAAAACCCCGGTCCTGC
CATGGTGAGCAAGGGCGAGGAGCTGTTCACCGGGGTGGTGCCCATCCTGGTCGAG
CTGGACGGCGACGTAAACGGCCACAAGTTCAGCGTGTCCGGCGAGGGCGAGGGC
GATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAGC
CGCTACCCCGACCACATGAAGCAGCACGACTTCTTCAAGTCCGCCATGCCCGAAG
GCTACGTCCAGGAGCGCACCATCTTCTTCAAGGACGACGGCAACTACAAGACCCG
CGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGAA
CTTCAAGATCCGCCACAACATCGAGGACGGCAGCGTGCAGCTCGCCGACCACTAC
CAGCAGAACACCCCCATCGGCGACGGCCCCGTGCTGCTGCCCGACAACCACTACC
TGAGCACCCAGTCCGCCCTGAGCAAAGACCCCAACGAGAAGCGCGATCACATGGT
CCTGCTGGAGTTCGTGACCGCCGCCGGGATCACTCTCGGCATGGACGAGCTGTAC
AAGTAATAAGCTCGCGTGGTACCTCTAGAGTCGACCCGGGCGGCCTCGAGGACGG
GGTGAACTACGCCTGAGGATCCGATCTTTTTCCCTCTGCCAAAAATTATGGGGACA
TCATGAAGCCCCTTGAGCATCTGACTTCTGGCTAATAAAGGAAATTTATTTTCATT
GCAATAGTGTGTTGGAATTTTTTGTGTCTCTCACTCGGAAGCAATTCGTTGATCTG
AATTTCGACCACCCATAATACCCATTACCCTGGTAGATAAGTAGCATGGCGGGTTA
ATCATTAACTACAAGGAACCCCTAGTGATGGAGTTGGCCACTCCCTCTCTGCGCGC
TCGCTCGCTCACTGAGGCCGGGCGACCAAAGGTCGCCCGACGCCCGGGCTTTGCC
CGGGCGGCCTCAGTGAGCGAGCGAGCGCGCAGCCTTAATTAACCTAATTCACTGG
CCGTCGTTTTACAACGTCGTGACTGGGAAAACCCTGGCGTTACCCAACTTAATCGC
CTTGCAGCACATCCCCCTTTCGCCAGCTGGCGTAATAGCGAAGAGGCCCGCACCG
ATCGCCCTTCCCAACAGTTGCGCAGCCTGAATGGCGAATGGGACGCGCCCTGTAG
CGGCGCATTAAGCGCGGCGGGTGTGGTGGTTACGCGCAGCGTGACCGCTACACTT
GCCAGCGCCCTAGCGCCCGCTCCTTTCGCTTTCTTCCCTTCCTTTCTCGCCACGTTC
GCCGGCTTTCCCCGTCAAGCTCTAAATCGGGGGCTCCCTTTAGGGTTCCGATTTAG
TGCTTTACGGCACCTCGACCCCAAAAAACTTGATTAGGGTGATGGTTCACGTAGTG
GGCCATCGCCCTGATAGACGGTTTTTCGCCCTTTGACGTTGGAGTCCACGTTCTTTA
ATAGTGGACTCTTGTTCCAAACTGGAACAACACTCAACCCTATCTCGGTCTATTCT
TTTGATTTATAAGGGATTTTGCCGATTTCGGCCTATTGGTTAAAAAATGAGCTGAT
TTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTC
AAATATGTATCCGCTCATGAGACAATAACCCTGATAAATGCTTCAATAATATTGAA
AAAGGAAGAGTATGAGTATTCAACATTTCCGTGTCGCCCTTATTCCCTTTTTTGCG
GCATTTTGCCTTCCTGTTTTTGCTCACCCAGAAACGCTGGTGAAAGTAAAAGATGC
TGAAGATCAGTTGGGTGCACGAGTGGGTTACATCGAACTGGATCTCAACAGCGGT
AAGATCCTTGAGAGTTTTCGCCCCGAAGAACGTTTTCCAATGATGAGCACTTTTAA
AGTTCTGCTATGTGGCGCGGTATTATCCCGTATTGACGCCGGGCAAGAGCAACTCG
GTCGCCGCATACACTATTCTCAGAATGACTTGGTTGAGTACTCACCAGTCACAGAA
AAGCATCTTACGGATGGCATGACAGTAAGAGAATTATGCAGTGCTGCCATAACCA
TGAGTGATAACACTGCGGCCAACTTACTTCTGACAACGATCGGAGGACCGAAGGA
GCTAACCGCTTTTTTGCACAACATGGGGGATCATGTAACTCGCCTTGATCGTTGGG
AACCGGAGCTGAATGAAGCCATACCAAACGACGAGCGTGACACCACGATGCCTGT
AGCAATGGCAACAACGTTGCGCAAACTATTAACTGGCGAACTACTTACTCTAGCTT
CCCGGCAACAATTAATAGACTGGATGGAGGCGGATAAAGTTGCAGGACCACTTCT
GCGCTCGGCCCTTCCGGCTGGCTGGTTTATTGCTGATAAATCTGGAGCCGGTGAGC
GTGGGTCTCGCGGTATCATTGCAGCACTGGGGCCAGATGGTAAGCCCTCCCGTATC
GTAGTTATCTACACGACGGGGAGTCAGGCAACTATGGATGAACGAAATAGACAGA
TCGCTGAGATAGGTGCCTCACTGATTAAGCATTGGTAACTGTCAGACCAAGTTTAC
TCATATATACTTTAGATTGATTTAAAACTTCATTTTTAATTTAAAAGGATCTAGGTG
AAGATCCTTTTTGATAATCTCATGACCAAAATCCCTTAACGTGAGTTTTCGTTCCAC
TGAGCGTCAGACCCCGTAGAAAAGATCAAAGGATCTTCTTGAGATCCTTTTTTTCT
GCGCGTAATCTGCTGCTTGCAAACAAAAAAACCACCGCTACCAGCGGTGGTTTGTT
TGCCGGATCAAGAGCTACCAACTCTTTTTCCGAAGGTAACTGGCTTCAGCAGAGCG
CAGATACCAAATACTGTTCTTCTAGTGTAGCCGTAGTTAGGCCACCACTTCAAGAA
CTCTGTAGCACCGCCTACATACCTCGCTCTGCTAATCCTGTTACCAGTGGCTGCTG
CCAGTGGCGATAAGTCGTGTCTTACCGGGTTGGACTCAAGACGATAGTTACCGGA
TAAGGCGCAGCGGTCGGGCTGAACGGGGGGTTCGTGCACACAGCCCAGCTTGGAG
CGAACGACCTACACCGAACTGAGATACCTACAGCGTGAGCTATGAGAAAGCGCCA
CGCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGGG
GGCGGAGCCTATGGAAAAACGCCAGCAACGCGGCCTTTTTACGGTTCCTGGCCTTT
TGCTGGCCTTTTGCTCACATGTTCTTTCCTGCGTTATCCCCTGATTCTGTGGATAAC
CGTATTACCGCCTTTGAGTGAGCTGATACCGCTCGCCGCAGCCGAACGACCGAGC
GCAGCGAGTCAGTGAGCGAGGAAGCGGAAGAGCGCCCAATACGCAAACCGCCTC
TCCCCGCGCGTTGGCCGATTCATTAATGCAGCTGGCACGACAGGTTTCCCGACTGG
AAAGCGGGCAGTGAGCGCAACGCAATTAATGTGAGTTAGCTCACTCATTAGGCAC
CCCAGGCTTTACACTTTATGCTTCCGGCTCGTATGTTGTGTGGAATTGTGAGCGGA
TAACAATTTCACACAGGAAACAGCTATGACCATGATTACGCCAGATTTAATTAAG
GCCTTAATTAGG

Methods for obtaining recombinant AAVs having a desired capsid protein are well known in the art. (See, for example, US 2003/0138772), the contents of which are incorporated herein by reference in their entirety). Typically, the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the present disclosure provides rAAV vector transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. In some embodiments, a host cell is a bacterial cell, yeast cell, insect cell (519), or a mammalian (e.g., human, rodent, non-human primate, etc.) cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. In some embodiments, the host cell in accordance with the present disclosure is a cardiomyocyte.

In some embodiments, the polypeptides or the nucleic acids (e.g., mRNAs, viral vectors, or rAAV) encoding the polypeptide are formulated in compositions (e.g., pharmaceutical compositions) for administration to a subject for treating arrhythmia. In some embodiments, the composition further comprises a pharmaceutically acceptable carrier.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Suitable carriers may be readily selected by one of skill in the art in view of the indication for which the composition (e.g., pharmaceutical composition) is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Typically, the compositions (e.g., pharmaceutical compositions) may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, the compositions comprise any one of the rAAVs described herein. In some embodiments, these compositions are formulated to reduce aggregation of AAV particles in the composition, particularly where high rAAV concentrations are present (e.g., ˜1013 GC/ml or more). Methods for reducing aggregation of rAAVs are well known in the art and include, for example, addition of surfactants, pH adjustment, salt concentration adjustment, etc. (See, e.g., Wright F R, et al., Molecular Therapy (2005) 12, 171-178, the contents of which are incorporated herein by reference.)

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For topical administration, the pharmaceutical composition can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The polypeptides, nucleic acids, rAAV, or pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active agents in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Delivery vehicles such as liposomes, nanocapsules, microparticles, microspheres, lipid particles, vesicles, and the like, may be used for the introduction of the compositions of the present disclosure into suitable host cells. In particular, the nucleic acids, proteins, or rAAVs may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids, proteins, or the rAAVs disclosed herein. The formation and use of liposomes are generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the active agents may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

The compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Other aspects of the present disclosure provide uses of any one of the polypeptides, nucleic acids, the rAAV, or the composition described herein for use in treating arrhythmia. In some embodiments, the method of treating arrhythmia comprises administering to a subject in need thereof an effective amount of a recombinant adeno-associated virus (rAAV), wherein the rAAV comprises a capsid protein (e.g., a capsid protein of serotype AAV9) and a nucleotide sequence encoding a polypeptide comprising a C-terminal domain of MYBPC3 (e.g., the polypeptide of any one of SEQ ID NOs: 1-16).

In its broadest sense, the terms “treatment” or “to treat” refer to both therapeutic and prophylactic treatments. If the subject is in need of treatment of a disease (e.g., arrhythmia), “treating the condition” refers to ameliorating, reducing or eliminating one or more symptoms associated with the or preventing any further progression of the disease (e.g., arrhythmia). If the subject in need of treatment is one who is at risk of having arrhythmia, then treating the subject refers to reducing the risk of the subject having arrhythmia or preventing the subject from developing arrhythmia.

A subject shall mean a human or vertebrate animal or mammal including but not limited to a rodent, e.g., a rat or a mouse, dog, cat, horse, cow, pig, sheep, goat, turkey, chicken, and primate, e.g., monkey. The methods of the present disclosure are useful for treating a subject in need thereof.

The term “therapeutically effective amount” of the present disclosure refers to the amount necessary or sufficient to realize a desired biologic effect. For example, a therapeutically effective amount of the polypeptide or nucleic acid encoding such associated with the present disclosure may be that amount sufficient to ameliorate one or more symptoms of arrhythmia. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compound associated with the present disclosure without necessitating undue experimentation.

In some embodiments, an “effective amount” of an rAAV is an amount sufficient to target infect an animal, target a desired tissue (e.g., heart tissue). The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some embodiments, a dosage between about 10¹³ to 10¹⁵ rAAV genome copies is appropriate.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., delivery to the heart), oral, inhalation (including intranasal and intratracheal delivery), intraocular, intravenous, intramuscular, subcutaneous, intradermal, intratumoral, and other parental routes of administration. Routes of administration may be combined, if desired.

The polypeptides, nucleic acids, rAAVs, and compositions comprising such of the disclosure may be delivered to a subject in compositions according to any appropriate methods known in the art. For example, an rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the polypeptides, nucleic acids, rAAVs, and compositions to a mammalian subject may be by, for example, intramuscular injection or by administration into the bloodstream of the mammalian subject. Administration into the bloodstream may be by injection into a vein, an artery, or any other vascular conduit. In some embodiments, the polypeptides, nucleic acids, rAAVs, and compositions as described in the disclosure are administered by intravenous injection. In some embodiments, the polypeptides, nucleic acids, rAAVs, and compositions are administered by intramuscular injection. In some embodiments, the polypeptides, nucleic acids, rAAVs, and compositions are administered by injection into the heart. In some embodiments, the polypeptides, nucleic acids, rAAVs, and compositions are delivered to a cardiomyocyte in the subject.

In some embodiments, a dose of the polypeptides, nucleic acids, rAAVs, or compositions are administered to a subject by intramuscular injection no more than once per calendar day (e.g., a 24-hour period). In some embodiments, a dose of the polypeptides, nucleic acids, rAAVs, or compositions are administered by intramuscular injection to a subject no more than once per 2, 3, 4, 5, 6, or 7 calendar days. In some embodiments, a dose of the polypeptides, nucleic acids, rAAVs, or compositions is administered to a subject no more than once per calendar week (e.g., 7 calendar days). In some embodiments, a dose of the polypeptides, nucleic acids, rAAVs, or compositions is administered to a subject no more than bi-weekly (e.g., once in a two-calendar week period). In some embodiments, a dose of rAAV is administered to a subject no more than once per calendar month (e.g., once in 30 calendar days). In some embodiments, a dose of the polypeptides, nucleic acids, rAAVs, or compositions is administered to a subject no more than once per six calendar months. In some embodiments, a dose of the polypeptides, nucleic acids, rAAVs, or compositions is administered to a subject no more than once per calendar year (e.g., 365 days or 366 days in a leap year). In some embodiments, a dose of the polypeptides, nucleic acids, rAAVs, or compositions is administered to a subject as single dose therapy.

The disorders that may be treated using the methods described herein are associated with abnormal ryanodine receptor type 2 (RYR2) function. In some embodiments, the abnormal RYR2 function is caused by one or more (e.g., 1, 2, 3, 4, 5, or more) mutations in RYR2. In some embodiments, the abnormal RYR2 function (e.g., caused by mutations in RYR2) is associated with excessive (e.g., at least 20%, at least 50%, at least 100%, at least 2-fold, at least 10-fold, at least 100-fold or more) diastolic Ca²⁺ release in cardiomyocytes in the subject. Mutations in RYR2 that cause excessive diastolic Ca²⁺ release in cardiomyocytes are known in the art, e.g., as described in Jiang et al., PNAS Aug. 31, 2004 101 (35) 13062-13067; Liu et al., PLoS One. 2017; 12(9): e0184177; and Postma et al., J Med Genet. November; 42(11):863-70, incorporated herein by reference.

In some embodiments, the disorder associated with abnormal RYR2 function is arrhythmia. In some embodiments, the arrhythmia is inherited or acquired. In some embodiments, the inherited arrhythmia is Catecholaminergic Polymorphic Ventricular Tachycardia (CPVT). In some embodiments, the CPVT is associated with a mutation in RYR2. In some embodiments, the acquired arrhythmia is a ventricular arrhythmia or a supraventricular arrhythmia. In some embodiments, the ventricular arrhythmia is ventricular tachycardia, ventricular fibrillation, or premature ventricular contraction. In some embodiments, the supraventricular arrhythmia is atrial fibrillation, atrial flutter, atrial tachycardia, premature atrial contraction, or paroxysmal supraventricular tachycardia. In some embodiments, the disorder associated with abnormal RYR2 function is heart failure.

In some embodiments, administering the polypeptide, the nucleic acid, or the rAAV reduces the excessive diastolic Ca²⁺ release (e.g., by at least 20%, at least 50%, or at least 90%) in cardiomyocytes in the subject. In some embodiments, administering the polypeptide, the nucleic acid, or the rAAV restores the diastolic Ca²⁺ release to a normal level in cardiomyocytes in the subject. In some embodiments, the normal level is the level of diastolic Ca²⁺ release in a healthy subject.

### EXAMPLES

### Example 1

CPVT (Catecholaminergic Polymorphic Ventricular Tachycardia) is a malignant inherited arrhythmia in which patients are at risk for lethal arrhythmias during exercise¹. CPVT has an estimated prevalence of 1:10000 and causes about 15% of autopsy negative cases of sudden unexplained death in the young². 60% of CPVT cases are caused by mutations in ryanodine receptor type 2 (RYR2)^{1,3}, the major intracellular Ca²⁺ release channel of cardiomyocytes. Within RYR2, over 160 different mutations, clustered within 4 “hotspot” regions of the coding sequence⁴, are known to cause CPVT. Currently CPVT is not adequately treated by available options, and patients continue to suffer from sudden death or aborted sudden death, as well as morbidities arising from current therapies⁵. Therefore the immediate proof-of-concept market space are patients with CPVT whose response to medical management is sub-optimal. Ultimately, it is anticipated that the gene therapy approach could become standard treatment for CPVT.

Mutations in CPVT interfere with normal cardiomyocyte Ca²⁺ handling. With each heartbeat, Ca²⁺ levels rise in systole, signaling sarcomeres to contract, and decline in diastole, causing sarcomeres to relax. These changes in cytoplasmic Ca²⁺ concentration are initiated by depolarization of the plasma membrane, which opens the L-type Ca²⁺ channel to allow a small amount of extracellular Ca²⁺ to enter the cell. This Ca²⁺ entry stimulates RYR2, located on the sarcoplasmic reticulum, to open and release much more Ca²⁺. This Ca²⁺-induced Ca²⁺ release rapidly increases cytosolic Ca²⁺, which coordinates sarcomere contraction. Time-dependent closure of the L-type Ca²⁺ channel and RYR2, along with active return of cytosolic Ca²⁺ to the sarcoplasmic reticulum by an ATP-dependent pump (SERCA2A), return Ca²⁺ concentrations to a low level in diastole. A small amount of Ca²⁺ returns to the extracellular space via the Na⁺/Ca²⁺ exchanger, NCX. CPVT mutations cause excessive diastolic Ca²⁺ release through RYR2. The elevated diastolic Ca²⁺ drives greater Na⁺/Ca²⁺ exchange. Since this exchange is electrogenic, elevated exchange results in membrane depolarization (after-depolarizations), which can result in another action potential (“triggered activity”) or create heterogeneity of repolarization that can cause arrhythmic impulse propagation (“re-entry”)^{6,7}.

The mechanism of action of the therapeutic described herein is to limit the excessive activity of RYR2, which is central to the pathogenesis of CPVT. Importantly, dysfunction of RYR2 is a final common pathway of many types of heart disease, and therefore it is likely that the indications for this anti-arrhythmic therapy could be expanded to include other types of inherited or acquired cardiomyopathy, including atrial fibrillation⁸ (prevalence, 1% of population and 9% of patients over 80 years of age).

Patients with CPVT are imperfectly treated by current medical and surgical options^{5,9,10}. The current medical options have substantial side effects and afford incomplete protection.

Our current medical option is exercise restriction. Exercise restriction is difficult in children and adolescents, and limiting exercise has lifelong psychosocial and medical implications. The long-term benefits of exercise are increasingly recognized and associated with cardiovascular, metabolic, and inflammatory disorders and the lifetime risk of breast, endometrial and colon malignancies.¹¹⁻¹³

Another current option is utilizing high dose beta-blockers. High dose beta-blockade is frequently difficult to tolerate due to effects on overall energy level and mood. As a result, non-compliance with beta-blockers, or sub-therapeutic dosing, is common. In a recent study, treatment failure (syncope or cardiac arrest) occurred in 25% to 33% of patients managed primarily with beta-blockers^{5,14}. Suboptimal dosing and non-adherence to prescribed therapy occurred in 41% and 48% of these treatment failures, respectively⁵.

Another current medical option is flecainide. The combination of beta-blocker plus flecainide, a sodium channel blocker, has been found to be effective for patients with CPVT15. In adult heart disease trials, flecainide had substantial pro-arrhythmic effects and increased mortality¹⁶. Whether or not flecainide increases long term survival in CPVT is not known. In acute exercise testing, 76% of patients responded to flecainide, and 24% did not¹⁷. In a retrospective study with limited follow-up (median 1.7 years), flecainide appeared promising, although 38% of patients had persistent symptoms⁵.

Yet another current medical option is left cardiac sympathetic denervation (LCSD). Surgical interruption of the left cervical sympathetic chain reduces adrenergic stimulation to the heart and has been beneficial to some CPVT patients who have breakthrough arrhythmias on medical management. LCSD should be performed at a specialized center, and surgical complications such as Homer's syndrome are not uncommon. LCSD reduced frequency of cardiac events, but in a median 37-month follow-up, 24% of patients had at least one recurrent cardiac event¹⁸.

Still another current medical option is implanted cardiac defibrillators (ICDs). In children and adolescents with CPVT, ICD complications were common and associated with a high burden of shocks¹⁰. ICDs were effective in terminating ventricular fibrillation but not ventricular tachycardia⁹. Furthermore, ICD discharge in an awake patient results in catecholamine release that can precipitate further arrhythmia, leading to potentially fatal “electrical storm”. Recent evidence shows no survival benefit from ICDs for patients who present with cardiac arrest secondary to CPVT. For these reasons, ICD placement for CPVT should be avoided whenever possible, although this leaves patients dependent on medication with the associated issues of compliance and breakthrough events¹⁹.

CPVT remains a major cause of morbidity and mortality in otherwise healthy, functional children with very significant societal and economic costs despite the relative rarity of the disease. Repeated hospital visits for clinical assessment and procedures expose the patient and institution to significant costs.

The present disclosure proposes compositions and methods for treating CPVT. The composition comprises AAV-CTDP, in which adeno-associated virus with a cardiomyocyte-selective promoter expresses a peptide, CTDP (MYBPC3 C-terminus-derived peptide), that reduces the aberrant activity of RYR2, the underlying cause of arrhythmia in CPVT and many other inherited and acquired arrhythmias.

The target population are all patients with CPVT, although patients who failed medical management (breakthrough arrhythmias on beta-blockers and flecainide) are started with. The gene therapy vector are delivered by intravenous infusion as single dose treatment. The gene therapy method described herein reduces mortality and breakthrough arrhythmias, reduce the need for LCSD and ICDs, reduces or eliminate the need for high dose beta-blockers, and permit some level of exercise. These changes would vastly improve quality of life for CPVT patients. Successful gene therapy would reduce the impact on patient outcome of medical compliance, which is a difficult issue with life or death consequences in these teenage and young adult patients. These benefits are expected based on the preliminary determination of efficacy in a CPVT mouse model and in human iPSC-derived cardiomyocytes harboring CPVT mutations.

It is anticipated that the compositions and methods described herein could extend to other arrhythmias that are more common than CPVT in which abnormal Ca²⁺ release from RYR2 is central to disease pathogenesis²⁰. One likely expansion indication is atrial fibrillation, which affects 9% of patients 80 years of age and greater.

One potential alternative to AAV-mediated delivery of CTDP is delivery as a cell penetrating peptide. Compared to AAV gene therapy, peptide therapy has properties and cost more similar to a conventional pharmaceutical. However, peptide levels and cardiac specificity would likely be lower than for AAV gene therapy. Additionally, to be clinically effective, the product would need to be orally available, which could be a challenge for peptide therapy. For these reasons, the primary strategy is AAV gene therapy, with peptide-based therapy being a potential alternative that is contingent upon improvements in cell penetrating peptide technology.

### Results

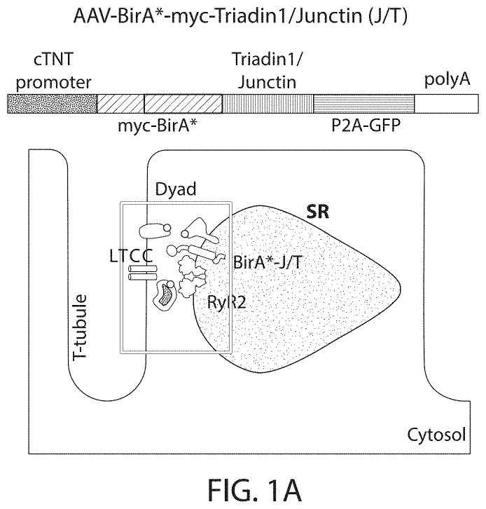

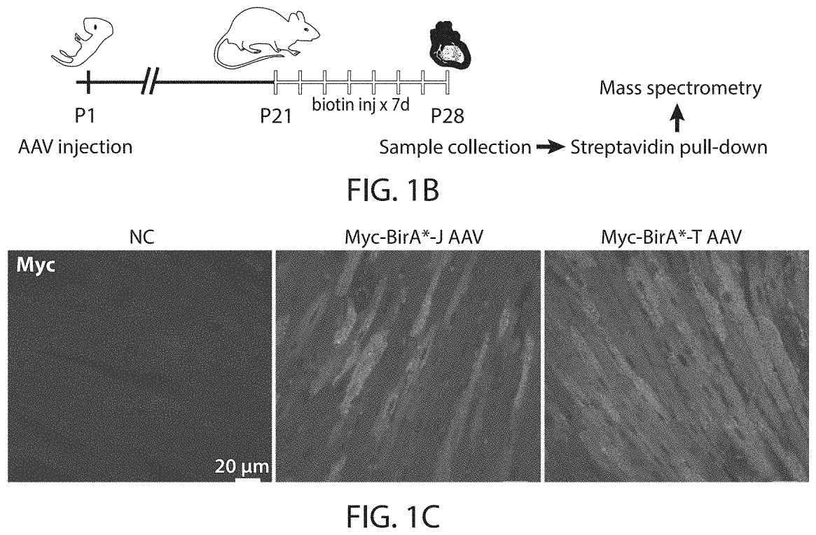

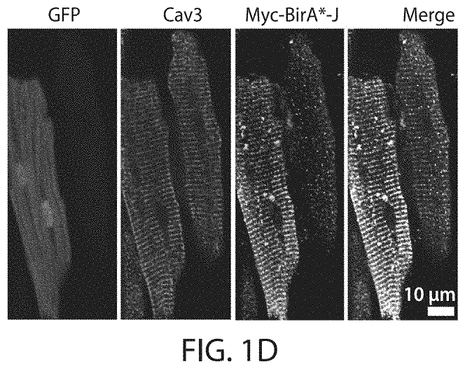

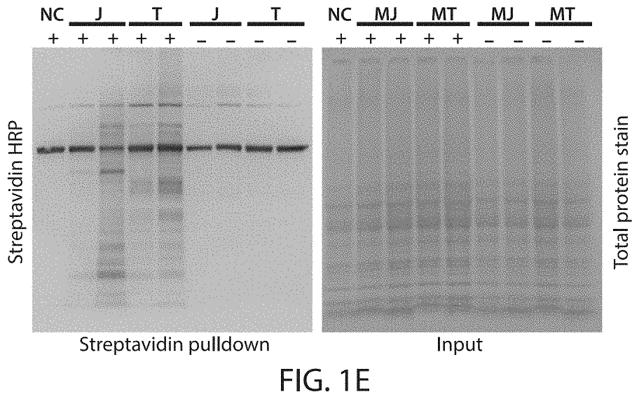

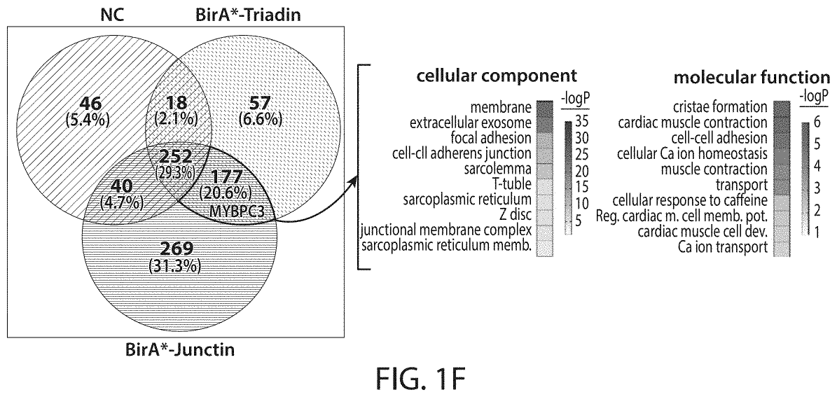

Proximity proteomics were performed to identify proteins that localize to dyads, where RYR2 is localized. This identified peptides derived from the C-terminus of MYBPC3, a sarcomere protein (FIGS. 1A-1F). Full length MYBPC3 localizes to a different portion of the sarcomere (the “A-band”). Consistent with this finding, MYBPC3-RYR2 interaction was previously noted in a yeast 2-hybrid screen²². Immunostaining using a monoclonal antibody specific to the most C-terminal domain of the protein, the C10 domain, demonstrated endogenous C10 co-localization with RYR2 (FIG. 2B). In control experiments it was shown that this monoclonal antibody does not yield significant immunofluorescent signal in MYBPC3 KO mice. Proximity of MYBPC3-C10 and RYR2 was further confirmed using the proximity ligation assay (PLA), an in situ assay for interaction between two proteins (FIGS. 2C and 2D).

To determine the functional significance of this interaction, AAV was developed to deliver portions of the MYBPC3 C-terminus to the mouse heart. MYBPC3 is composed of several immunoglobulin-like and fibronectin-like domains, labeled C1-C10 (FIG. 2A). The distribution of C10 was compared to full length MYBPC3, both delivered by AAV, and it was confirmed that these proteins localize to different sites: C10 localized in a pattern consistent with RYR2 near sarcomere Z lines (where dyads are located), whereas the full-length protein localized to MYBPC3's well established location within the sarcomere A band (FIG. 2E).

An important consideration for the feasibility of human gene therapy is the percent of cardiomyocytes that need to be transduced to achieve efficacy. A parallel question is whether partial myocardial transduction and resulting myocardial heterogeneity might be pro-arrhythmic. Although answers to these questions specifically with respect to AAV-MYBPC3 have yet to be determined, results from other gene therapy studies for CPVT are informative. In AAV gene replacement therapy for CPVT caused by CASQ2 deficiency (the autosomal recessive form of CPVT), Priori and colleagues reported therapeutic efficacy and no pro-arrhythmia in mice with ˜40% cardiomyocyte transduced^{24,25}. Similarly, in the report of AAV-mediated CaMKII inhibition to treat CPVT caused by RYR2 mutation, therapeutic efficacy without pro-arrhythmia was observed in mice with 50% cardiomyocytes transduced²¹. Formal dose-response experiments are underway to determine the minimum transduction efficiency needed for efficacy; based on pilot experiments with low numbers of replicates, it is believed to be approximately 20%. The mechanism is likely based in a concept known as “source-sink mis-match”: Because cardiomyocytes are electrically connected to their neighbors, the activity of one cardiomyocyte is stabilized by its interactions with neighboring cells. For a cardiomyocyte to aberrantly depolarize, it needs to generate sufficient current to also depolarize neighboring cells. In this way, a low fraction of cardiomyocytes that are resistant to aberrant activity can stabilize a network of cells.

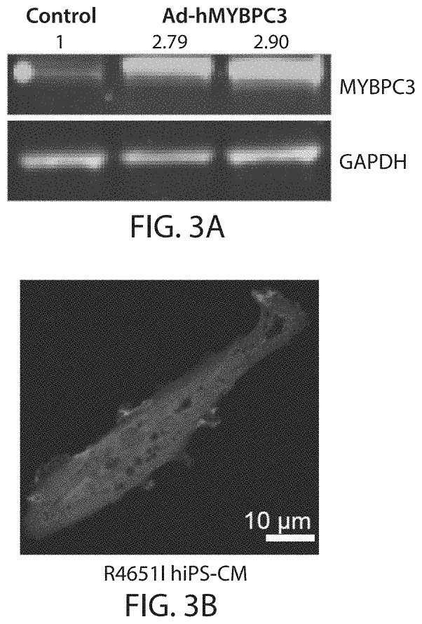

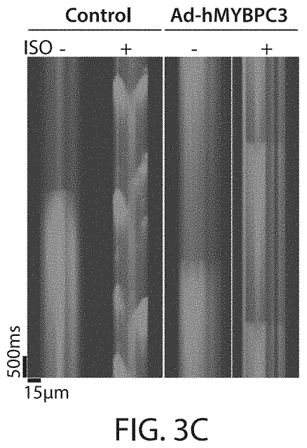

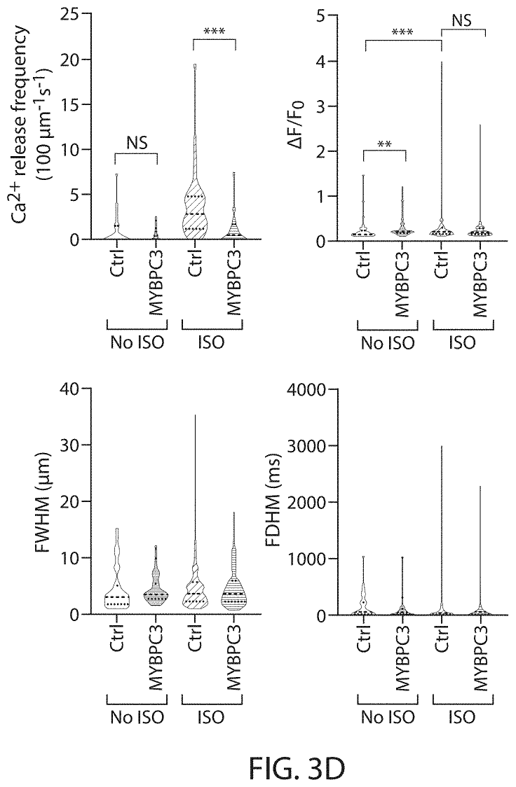

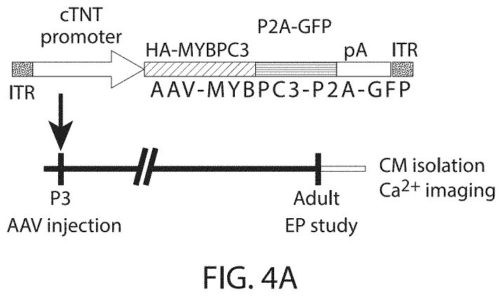

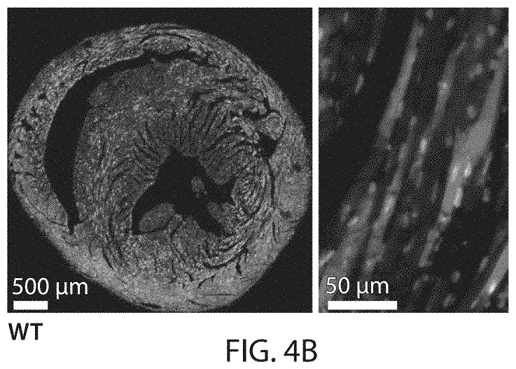

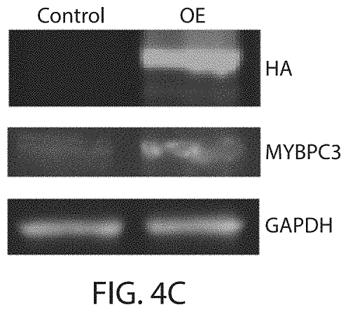

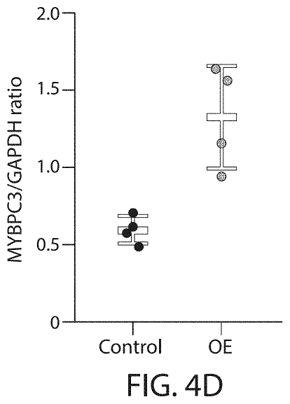

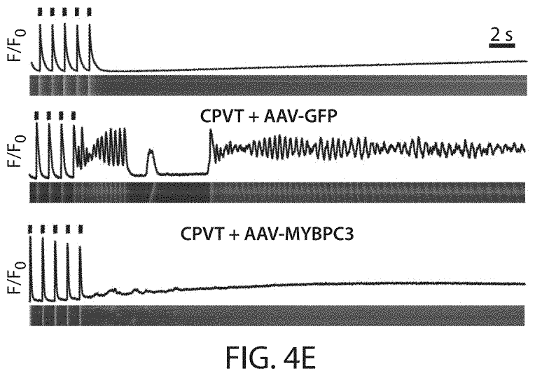

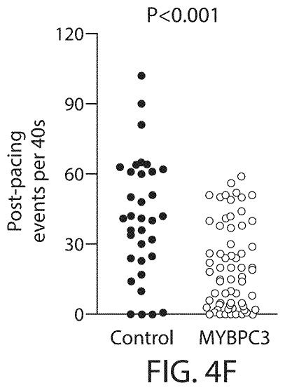

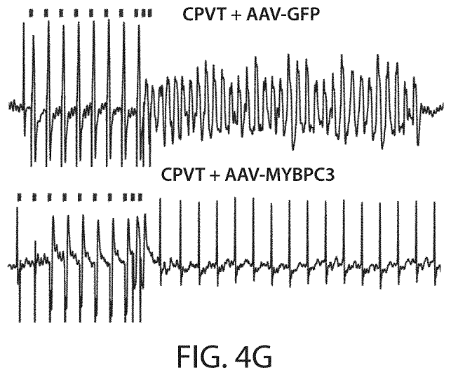

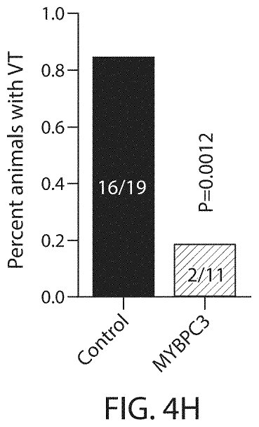

The effect of MYBPC3 on Ca²⁺ handling of human CPVT patient-derived iPSC-CMs was evaluated. MYBPC3 expression reduced the frequency of Ca²⁺ sparks in CPVT iPSC-CMs stimulated with isoproterenol, a beta-adrenergic agent (FIG. 3D). This demonstrates efficacy in human cells and an expertise in human iPSC-CM culture and characterization of Ca²⁺ handling in these cells.

Conducting dose response experiments with a therapeutic candidate vector without a reporter gene can make measurement of transduction efficiency difficult. However, this is a key parameter to scale dosing between species. To overcome this difficulty, RNA in situ hybridization methods were established in the laboratory. For example, for a separate project using AAV-TAZ to treat a mouse model of Barth syndrome, RNAscope RNA in situ hybridization was used to measure the fraction of cardiomyocytes that were transduced. This same technology are used here to measure transduction efficiency without relying on a reporter gene embedded in the therapeutic candidate vector.

Current standard of care has been effective at reducing the risk of cardiac arrest and death for CPVT patients. However, protection is incomplete and cardiac arrest and death continue to be a threat. Incomplete protection from current SOC is due to (1) intolerable side effects of current management, which result in non-compliance; and (2) failure to target the root cause of CPVT, dysfunction of RYR2. Exercise restriction, beta-blockers, and cardiac sympathetic denervation are designed to minimize pro-arrhythmic effects of beta-adrenergic signaling that trigger arrhythmia in CPVT patients. However, a recent retrospective study showed that about one fifth of cardiac events in CPVT were not provoked by an identifiable excitatory stimulus⁵, suggesting that removal of adrenergic signaling by itself may not be fully protective. The incomplete protection of many patients by exercise restriction, beta-blockers^{5,14}, and even surgical sympathetic denervation indicate that targeting this signaling pathway alone is insufficient¹⁸. Likewise, flecainide is incompletely protective—in acute testing, 24% of patients did not respond, and in short term follow-up, 38% of patients continued to have significant events while on flecainide¹⁷.

It is demonstrated herein that AAV-CTDP improved outcomes by addressing both of these problems with current standard of care. Both RYR2 and MYBPC3 are cardiac specific proteins, and the AAV will selectively direct expression to the heart. Therefore, minimal effects outside of cardiomyocytes are expected. CTDP directly interacts with RYR2 and reduces spontaneous Ca²⁺ release through mutant RYR2 channels. This mechanism of action on the affected channel is more direct than current strategies of beta-blockade or flecainide. Importantly, these strategies are likely to be complementary, so that a multi-layered strategy might be envisioned to afford maximal protection while minimizing side effects. For example, administration of AAV-CTDP could directly reduce aberrant RYR2 activity. Additional protection could be afforded by beta-blocker, perhaps at lower doses that are more easily tolerated, or by flecainide. If the therapy was highly effective, some patients could return to some level of physical activity, guided by wearable heart rate monitors.

In summary, the AAV-CTDP described herein might supplant current standard of care and be sufficient as monotherapy. At the least, AAV-CTDP is able to synergize with current standard of care and permit lower level beta-blockade and less stringent exercise restriction, so that patients can be better protected from risk of sudden death while reducing side effects and thereby enhancing compliance.

### Example 2

Next the therapeutic candidate vector design is optimized. These optimization experiments are performed in human iPSC-CMs and CPVT mouse (RYR2-R176Q/+ and RYR2-R4650I/+) adult CMs. There are two parameters to consider.

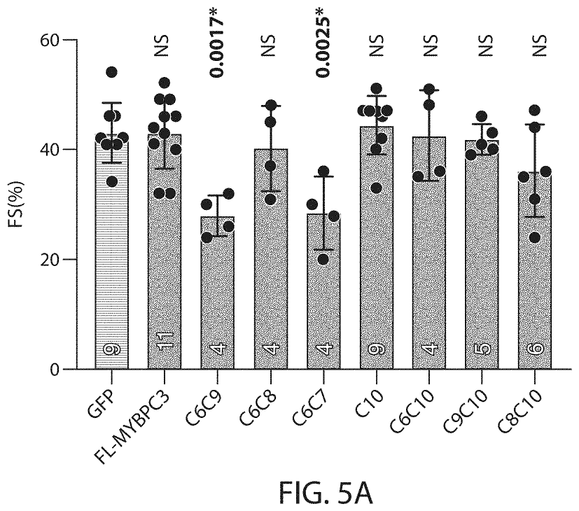

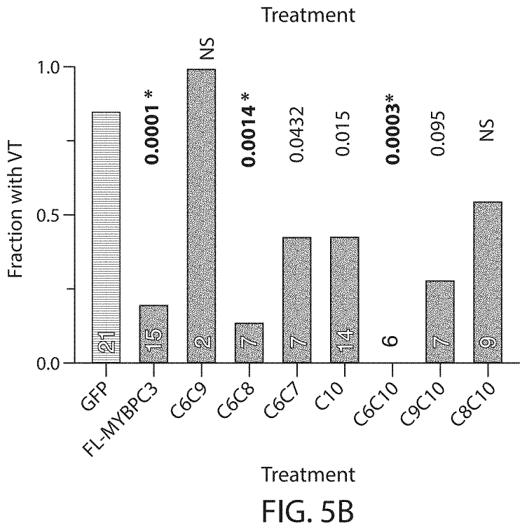

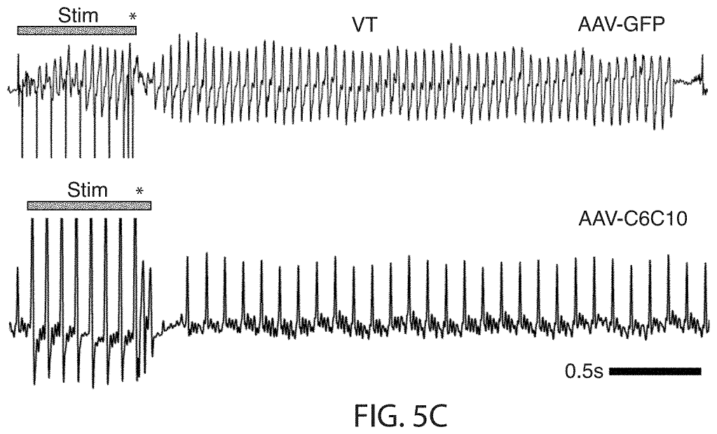

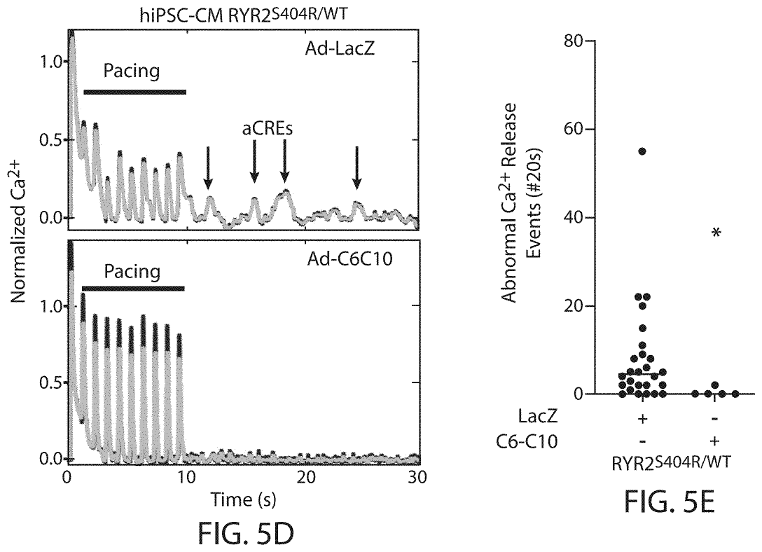

The first parameter to consider is the RYR2 inhibitory peptide. Preliminary data suggests that the C-terminus of MYBPC3, is effective in reducing the aberrant activity of RYR2 containing a CPVT mutation. AAV that express different C-terminal peptides (C6-C10, C6-C8, C8-10, C9-C10, C10, C6-C9, C7-C9, C8-C9, C9) were constructed. Initial in vitro data indicated that peptides comprising the C6-C8 and C6-C9, and the C10 domain bind to the same sub-cellular location as RYR2 (FIGS. 2A-2F). Peptide fragments comprising the C6, C7, C8, C9 and/or C10 domains were further tests for an ability to decrease VT in RYR2^{S404R/WT} mice. The data showed that C6-C8 and C6-C10 were the most effective at decreasing VT (FIGS. 5B-5C) and did not impair heart contraction (FIG. 5A-5C). The C6-C10 fragment was also shown to reduce CT using EKG (FIG. 5C) and decrease abnormal calcium signally (FIG. 5D-5E).

### Mapping of the Minimal Effective MYBPC3 Fragment

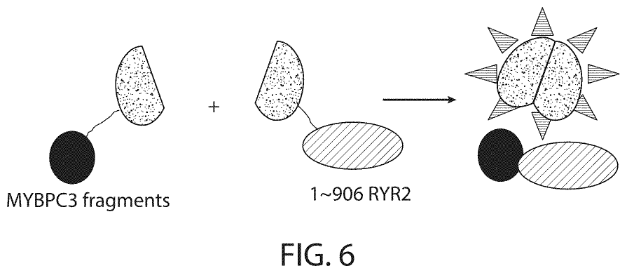

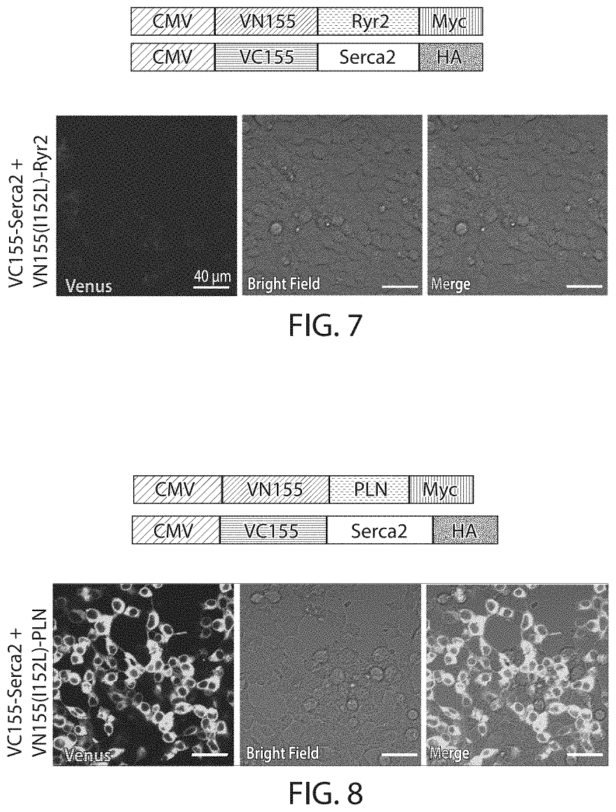

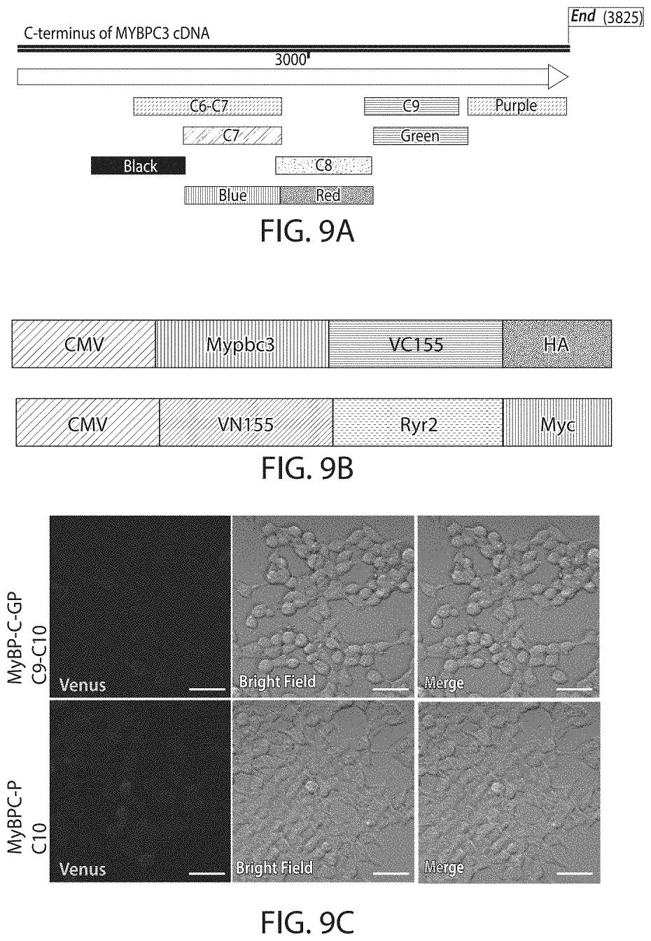

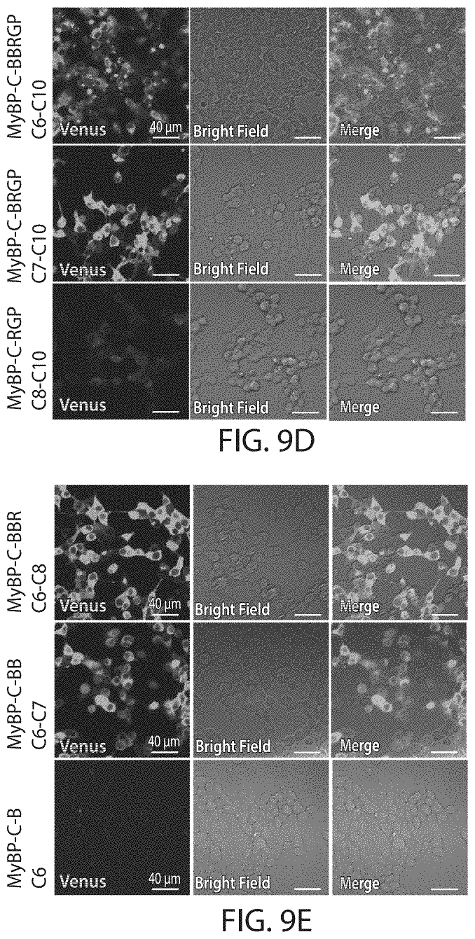

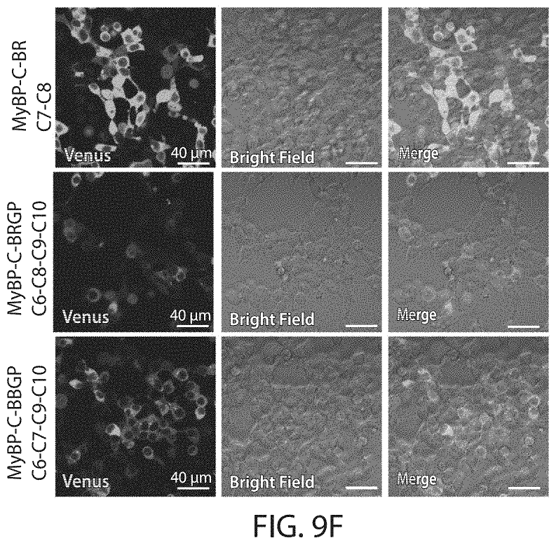

The fragments of MYPBC3 that interact with RYR2 were identified using a Biomolecular fluorescence complementation assay (BiFC) as outlined in FIG. 6. In the BiFC assay, MYBPC3 fragments and RYR2 were each fused to half of a Venus florescence protein. If a given MYBPC3 fragment interacted with RYR2 then the Venus halves come together, and a fluorescent signal is identified. MYBPC3 fragments were based on known domain structures (FIG. 9A) and outlined in Table 2. The PLN-Serca2 interaction was used as a positive control and the Serca-RYR2 interaction was used as a negative control for interaction in the BiFC (FIGS. 7-8).

TABLE 2
Proteins and protein fragments used in BiPC assay.

| Protein 1: | | | | | |
|---|---|---|---|---|---|
| MYBPC3 | Protein 2: | | | | |
| truncates (AA | RyR2 | Positive | Positive | Negative | Negative |
| positions) | truncate | Control | Control | Control | Control |
| C6C10 | mRyR2 1-906 | PLN | Serca2 | Junctin | Serca2 |
| (771-1274) | | | | | |
| C6C10 | | | | | |
| (871-1274) | | | | | |
| C6C10 | | | | | |
| (771-870, | | | | | |
| 971-1274) | | | | | |
| C6C10 | | | | | |
| (771-970, | | | | | |
| 1071-1274) | | | | | |
| C6C10 | | | | | |
| (771-1070, | | | | | |
| 1171-1274) | | | | | |
| C6C10 | | | | | |
| (771-1170) | | | | | |
| C6C10 | | | | | |
| (971-1274) | | | | | |
| C6C10 | | | | | |
| (1071-1274) | | | | | |
| C6C10 | | | | | |
| (1171-1274) | | | | | |
| C6C10 | | | | | |
| (771-1070) | | | | | |
| C6C10 | | | | | |
| (771-970) | | | | | |
| C6C10 | | | | | |
| (771-870) | | | | | |
| cDNA source | PCR | Synthesis | Mouse | Rat | Mouse |
| Annotation | Split-FP to the | Split-FP | Split-FP | Split-FP | Split-FP |
| | C terminus | to the N | to the N | to the N | to the N |
| | | terminus | terminus | terminus | terminus |

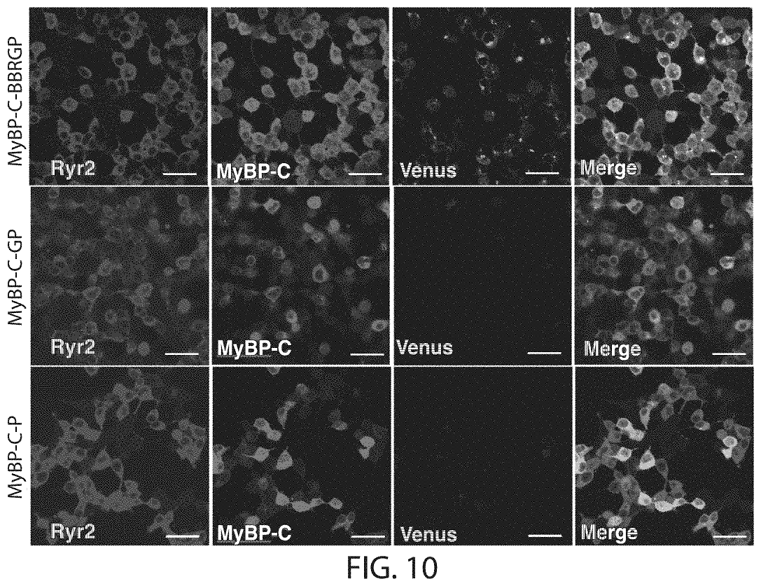

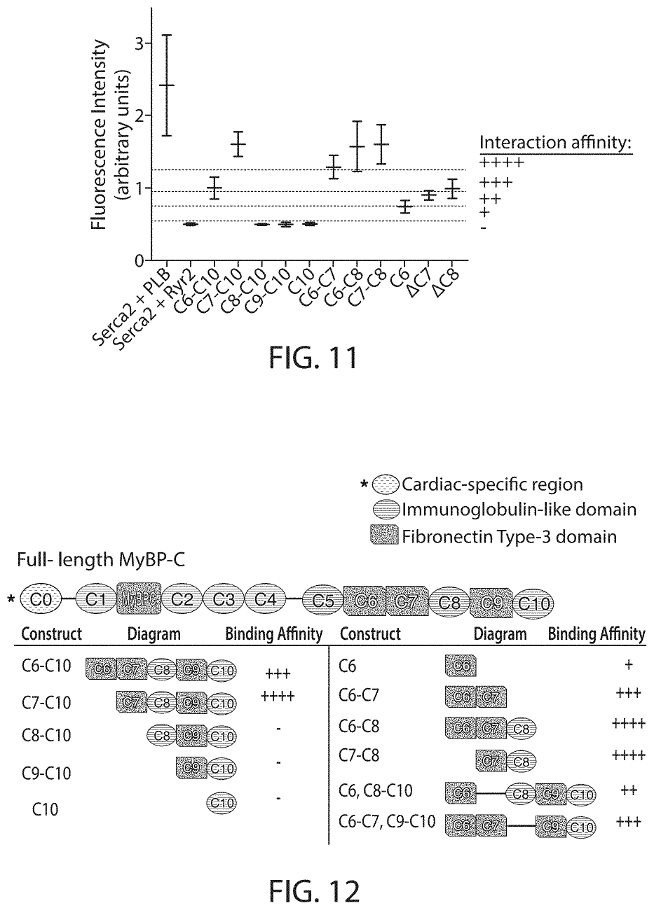

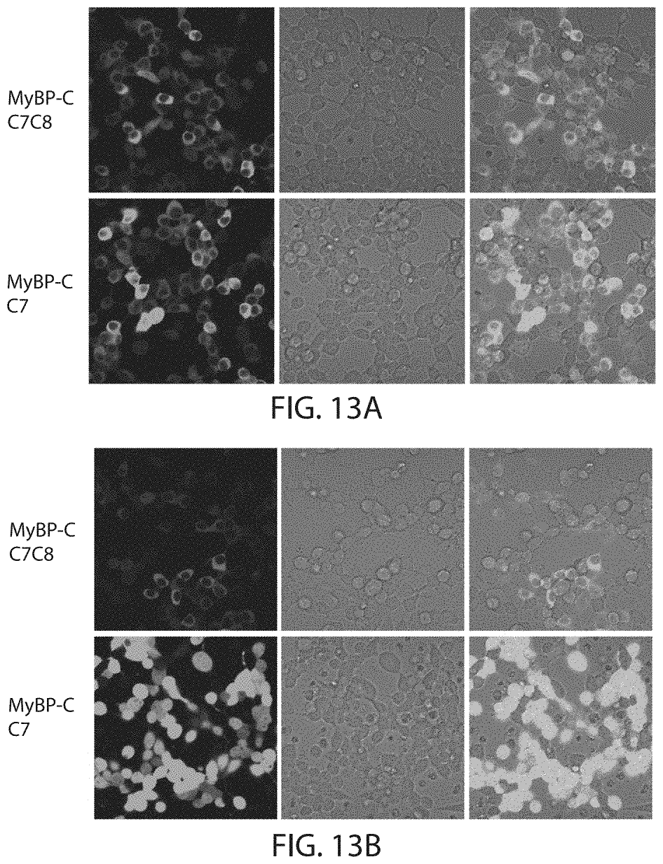

Results from the BiFC demonstrated that the C7 and C8 regions of MYBPC3 are the major contributor to the interaction between MYBPC3 and RYR2. Different fragments of MCBPC3 were test for binding to RYR2. Results from C9-C10, C10, C6-C10, C7-C10, and C8-C10 strongly suggested that the C7 and C8 regions both contribute to binding (FIGS. 9C-9D). The C6-C8 regions of MYBPC3 were then tested for binding to RYR2 and it was found that C6 fragment alone does not bind RYR2, but that C6-C7 and C6-C8 fragments did bind to RYR2 (FIG. 9E). Further experiments determined that C7-C8 are sufficient to bind RYR2 and that MYBPC3 fragments missing C7 or C8 could bind to RYR2, albeit with less affinity (FIG. 9F). The fluorescent images in FIGS. 9A-9F were quantified in FIG. 11 and further demonstrated that fragments containing C7 and/or C8 bind to RYR2 compared to fragments that do not have C7 and C8. Additional experiments showed that the interaction between MYBPC3 and RYR2 predominantly occurs through the C7 fragment (FIGS. 13A-13B). The binding efficacy of each MYBPC3 to RYR2 is summarized graphically in FIG. 12 with increasing numbers of “+++” indicating higher interaction affinity. It was also shown that non-interacting MYPBC3 domains are co-expressed with RYR2 and robustly expressed excluding technical failure of expression as the reason for low Venus signal (FIG. 10).

### Localization of AAV-Expressed MYBPC3 Fragments in Cardiomyocytes

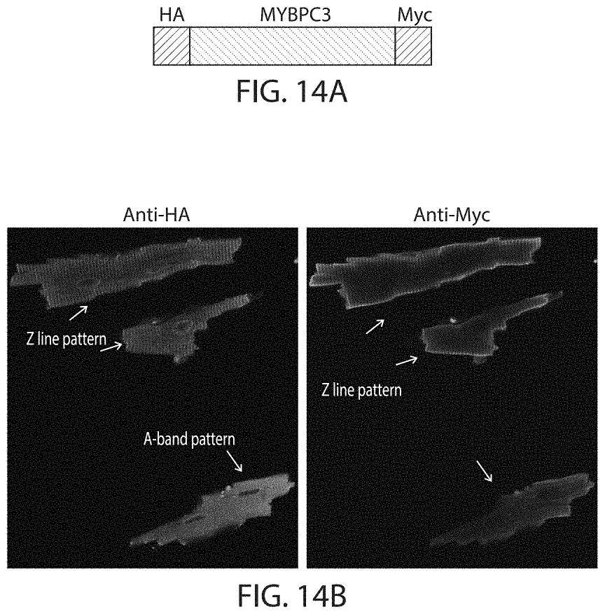

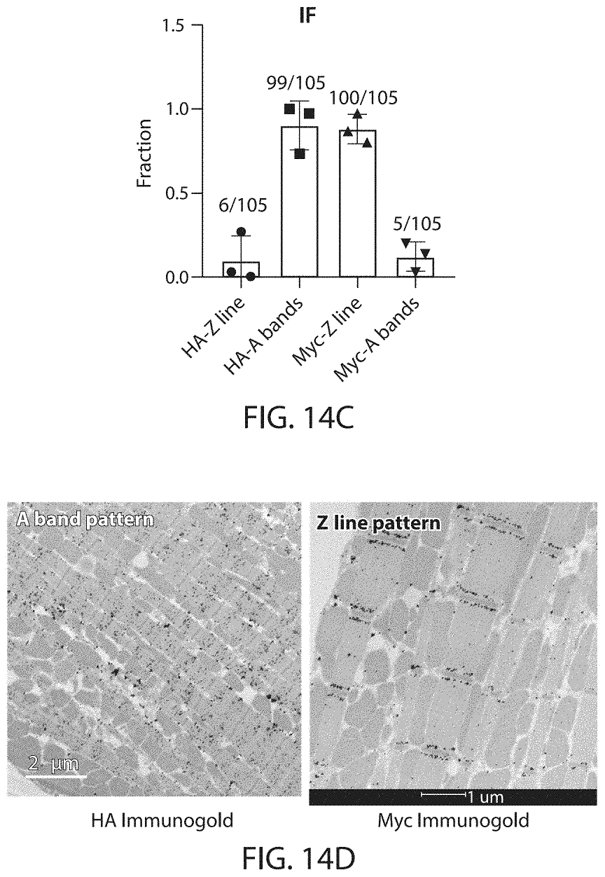

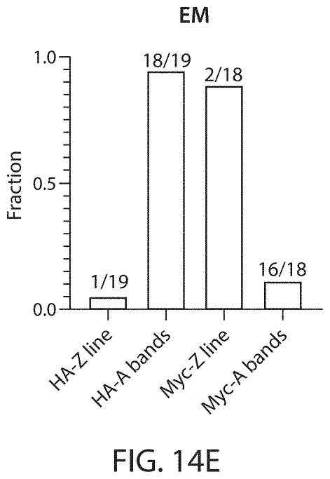

MYBPC3's established localization in cardiomyocytes is the A-band of sarcomeres. However, RYR2 is located in junctional SR/days, which are close to sarcomere Z-lines. Experiments were performed to determine if MYBCP3 fragments localize near the Z-line and therefore in the same region and RYR2. To do this, a MYBPC3 construct was made with a HA tag on the N-terminal and a Myc tag on the C-terminal (FIG. 14A). This construct was delivered by AAV to cardiomyocytes. It was observed that different cardiomyocytes in the same field of view had different staining patters for the HA-MYBPC3-Myc protein. Some cells had Z-line staining patterns whereas other cells had A-band staining patterns (FIG. 14B). Further analysis shows that HA containing fragments primarily bound to A-bands, whereas Myc containing fragments primarily bound to Z-lines (FIGS. 14C-14E). This suggested that MYBCP3 was being cleaved after administration to the cells.

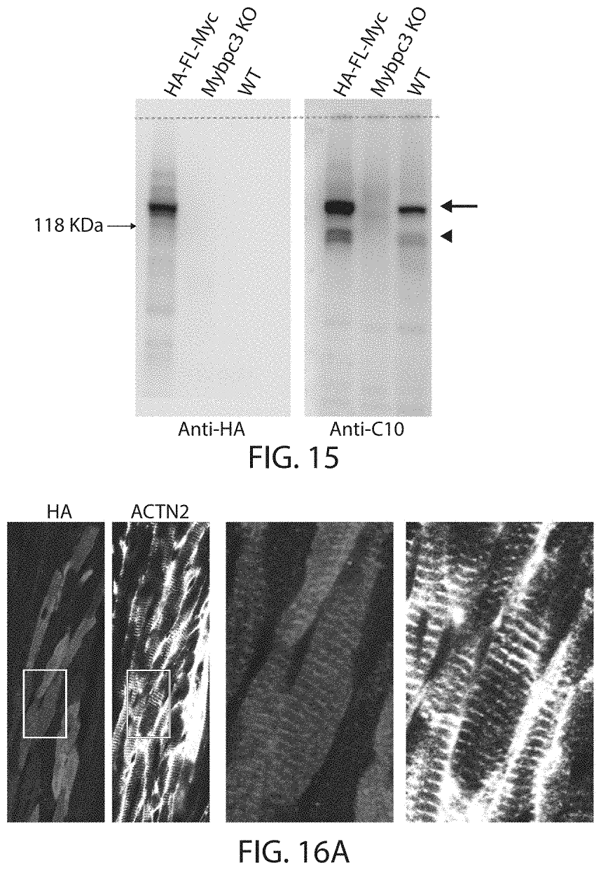

To test this, cardiomyocyte lysates from wild type, wild-type+HA-MYBPC3-MYC, and MYBPC3 KO hearts were probed using HA or C10 (monoclonal Ab that recognizes the C-terminal most domain of MYBPC3) antibody (FIG. 15). KO samples show that these antibodies do not recognize other proteins in the lysates. C10 antibody recognizes a full length (arrow) and a smaller protein (arrowhead), whereas the HA antibody recognizes only the full length protein. The smaller protein is present in both WT and WT+HA-MYBPC3-MYC, suggesting that a fraction of both exogenous and endogenous MYBPC3 is internally cleaved to yield a smaller protein that includes its C-terminal domain.

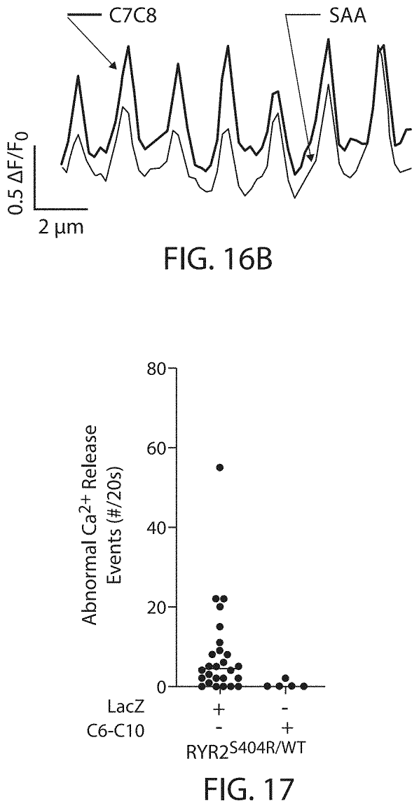

To determine if the C7-C8 fragment localized to Z-line patterns in cardiomyocytes in vivo, mice were treated with AAV-cTnT-HA-C7C8-P2A-GFP (SEQ ID NO: 78). Heart sections were stained with HA and ACTN2 (a Z-line marker). Confocal images and signal intensity along a line parallel to the cardiomyocyte long axis show that HA stain had a striated pattern that co-localized with Z-lines showing that the C7-C8 fragment localizes to the same location as the RYR2 protein in vivo (FIGS. 16A-16B).

Response of Human CPVT iPSC-CMs to Overexpression of MYBPC3

It was further demonstrated the C6-C10 MYBC3 fragment suppresses abnormal calcium release in human iPSC-CMs with CPVT caused by a RYR2-5404 mutation (FIG. 17) Cells were loaded with a Ca2+ sensitive dye and electrically paced at 1 Hz. The number of abnormal Ca2+ release events per 20 seconds was quantified. MYBPC3 suppressed abnormal Ca2+ release events in the CPVT mutant cells.

### Example 3

RYR2 is a tetramer with higher order clustering that is important for normal Ca²⁺-induced Ca²⁺ release. This structural organization suggests the possibility that multimerizing the MYBPC3-derived interacting protein may increase potency or efficacy. Using the minimal region required for anti-arrhythmic effect in vivo identified above (e.g., C7-C8 or C7), concatemers are generated in which 2 or 3 copies are separated by a flexible linker. The efficacy of these constructs is compared using in vitro and in vivo assays. The effect on cardiac function is also examined by echocardiography. The optimized therapeutic construct is named C-terminus derived peptide, “CTDP”. The second parameter to consider when optimizing the therapeutic candidate vector is the promoter used to drive cardiomyocyte expression. Promoters and enhancers are tested to identify the combination with maximal level of expression and cardiomyocyte selectivity. A massively parallel reporter assay was previously developed to test thousands of candidate enhancers in parallel³⁴, and this assay is currently being used to find the most potent and cardiac specific enhancers and promoters to drive expression from AAV.

These experiments are done with an AAV9 capsid because it is established as an efficient gene therapy vector in mice, and it has been used previously in an FDA-approved human product.

Next the therapeutic mechanism is evaluated. It is believed that the C-terminal region of MYBPC3 interacts with RYR2 and reduces diastolic Ca²⁺ flux. The effect of CTDP on RYR2WT and RYR2R176Q/+ diastolic Ca²⁺ flux is measured. RYR2-R176Q/+ and littermate control mice are treated with control AAV (AAV-GFP) or AAV-GFP-CTDP. 6-week old cardiomyocytes are isolated and diastolic sarcoplasmic reticulum Ca²⁺ leak are measured using an established protocol³³.

To further test if MYBPC3 directly interacts with RYR2, a heterologous expression system and planar lipid bilayers is used. RYR2 wild-type or RYR2R176Q expression plasmid are transfected into HEK293 cells, and endoplasmic reticulum vesicles are purified. The vesicles are used to seed a planar lipid bilayer. Ca²⁺ current through the bilayer is measured after treatment with increasing concentration of recombinant CTDP. CTDP normalizes Ca²⁺ release by RYR2R176Q.

Next, dose-response and toxicity studies in the mouse CPVT model is performed. Using the optimized therapeutic candidate, dose-response experiments are performed in CPVT mice to determine the minimum percent of cardiomyocytes that must be transduced to suppress arrhythmia. In preliminary experiments, dose finding and biodistribution studies with AAV-CTDP are performed. 4-week old mice are injected intravenously with AAV-CTDP or control (AAV-GFP). At 8 weeks, mice are euthanized and tissues (heart, lung, spleen, liver, kidney, testes/ovaries, skeletal muscle, and brain) will be collected for histological and molecular studies. Cryosections are analyzed for GFP expression. Heart samples are analyzed by RNAscope in situ hybridization to directly measure the fraction of cardiomyocytes transduced by AAV-CTDP. Molecular studies measure RNA expression of GFP or CTDP, and viral genome copies per host genome.

Having established viral doses that yield 10%, 30%, and 50% cardiomyocyte transduction, dose-response studies are performed next. Two different mouse CPVT models are used, RYR2-R176Q/+ and RYR2-R4650I/+. These CPVT mutations occur in different mutation hotspot regions at opposite ends of the protein. Use of both genotypes help to show that the treatment is effective against multiple different CPVT-causing RYR2 mutations. Both CPVT models and littermate control mice are studied. The mice are treated at 4 weeks of age with these three doses of AAV-CTDP, or with AAV-GFP at a dose that transduces 50% of cardiomyocytes. After 4 weeks, mice undergo echocardiography and then an electrophysiology study. The electrophysiology study involves insertion of an octapolar pacing/recording catheter through the right carotid and into the right ventricle. Mice are treated with adrenergic stimulation (isoproterenol plus epinephrine) and with programmed ventricular stimulation as recently described²¹. Following the electrophysiology study, mice are euthanized and tissues preserved for histological and molecular assays. These studies are performed blinded to genotype and treatment group. There are 10 animals per group, 3 genotypes, and 3 doses, plus one dose of the control vector. This study requires dosing and an electrophysiology study of 120 mice.

Next the efficacy in a rabbit CPVT model is tested. Mouse cardiac physiology is significantly different from human. For example, mouse heart rate is 10 times faster than human, and the heart mass is 2000 times smaller. In contrast, rabbit cardiac physiology is more similar to human—the rabbit heart rate is about 2 times faster than human, and the mass is about 10 times lower. Heart rate and size have important implications for expression of cardiac ion channels and for susceptibility to arrhythmia. The closer alignment between rabbit and human cardiac electrophysiology indicates that demonstration of efficacy and safety in the rabbit model would significantly de-risk the therapeutic strategy. The rabbit model is expensive both in terms of rabbit breeding and housing, and production of sufficient AAV. Therefore, initial dose finding studies are performed in mouse models as described and then validated in rabbit models.

A rabbit CPVT model (R4650I/+) is being developed. Control and treated CPVT rabbits are compared for arrhythmic response to catecholamine stimulation or to programmed ventricular stimulation.

In initial dose-finding and biodistribution studies using AAV-GFP, several doses of the therapeutic vector are tested, and transduction of heart and other tissues are measured, as described in task two above for mice. Juvenile rabbits (8 weeks old) are treated intravenously with AAV-GFP. Four weeks later, transduction and expression are measured in heart, lung, spleen, liver, kidney, testes/ovaries, skeletal muscle, and brain. Rabbits are treated with AAV-CTDP at a comparable dose to confirm equivalent cardiac transduction efficiency, using RNAscope in situ hybridization.

CPVT and littermate control rabbits are treated with the dose of virus that transduces cardiomyocytes to the level that is found to be effective in mice as described in task two. A third cohort of CPVT rabbits are not treated. Four weeks after treatment, rabbits undergo echocardiography and then an electrophysiology study. An electrophysiology study consist of surface EKG and intracardiac recording during adrenergic stress (isoproterenol plus epinephrine) and programmed ventricular stimulation. There are a total of 10 rabbits per group in three groups for a total of 30 rabbits.

Next the efficacy in human iPSC-CMs across a range of CPVT genotypes is tested. AAV-CTDP on iPSC-CMs are tested from patients with several different CPVT genotypes that map to each of the 4 CPVT mutation hotspot regions. AAV2 capsid can be used to transfect cultured cells. The efficacy of the therapeutic candidate are measured across genotypes, using Ca²⁺ spark frequency as the primary readout.

### REFERENCES

1. Venetucci L, Denegri M, Napolitano C, Priori S G. Inherited calcium channelopathies in the pathophysiology of arrhythmias. Nat Rev Cardiol. 2012; 9:561-575.
2. Tester D J, Spoon D B, Valdivia H H, Makielski J C, Ackerman M J. Targeted mutational analysis of the RyR2-encoded cardiac ryanodine receptor in sudden unexplained death: a molecular autopsy of 49 medical examiner/coroner's cases. Mayo Clin Proc. 2004; 79:1380-1384.
3. Roston T M, Yuchi Z, Kannankeril P J, Hathaway J, Vinocur J M, Etheridge S P, Potts J E, Maginot K R, Salerno J C, Cohen M I, Others. The clinical and genetic spectrum of catecholaminergic polymorphic ventricular tachycardia: findings from an international multicentre registry. Ep Europace. 2017; 20:541-547.
4. Priori S G, Chen S R W. Inherited dysfunction of sarcoplasmic reticulum Ca²⁺ handling and arrhythmogenesis. Circ Res. 2011; 108:871-883.
5. Roston T M, Vinocur J M, Maginot K R, Mohammed S, Salerno J C, Etheridge S P, Cohen M, Hamilton R M, Pflaumer A, Kanter R J, Potts J E, LaPage M J, Collins K K, Gebauer R A, Temple J D, Batra A S, Erickson C, Miszczak-Knecht M, Kubu\u s P, Bar-Cohen Y, Kantoch M, Thomas V C, Hessling G, Anderson C, Young M-L L, Cabrera Ortega M, Lau Y R, Johnsrude C L, Fournier A, Kannankeril P J, Sanatani S. Catecholaminergic polymorphic ventricular tachycardia in children: analysis of therapeutic strategies and outcomes from an international multicenter registry. Circ Arrhythm Electrophysiol. 2015; 8:633-642.
6. Liu M B, de Lange E, Garfinkel A, Weiss J N, Qu Z. Delayed afterdepolarizations generate both triggers and a vulnerable substrate promoting reentry in cardiac tissue. Heart Rhythm. 2015; 12:2115-2124.
7. Park S-J, Zhang D, Qi Y, Li Y, Lee K Y, Bezzerides V J, Yang P, Xia S, Kim S L, Liu X, Lu F, Pasqualini F S, Campbell P H, Geva J, Roberts A E, Kleber A G, Abrams D J, Pu W T, Parker K K. Insights Into the Pathogenesis of Catecholaminergic Polymorphic Ventricular Tachycardia From Engineered Human Heart Tissue. Circulation. 2019; 140:390-404.
8. Dobrev D, Wehrens X H T. Role of RyR2 phosphorylation in heart failure and arrhythmias: Controversies around ryanodine receptor phosphorylation in cardiac disease. Circ Res. 2014; 114:1311-9; discussion 1319.
9. Miyake C Y, Webster G, Czosek R J, Kantoch M J, Dubin A M, Avasarala K, Atallah J. Efficacy of implantable cardioverter defibrillators in young patients with catecholaminergic polymorphic ventricular tachycardia: success depends on substrate. Circ Arrhythm Electrophysiol. 2013; 6:579-587.
10. Roston T M, Jones K, Hawkins N M, Bos J M, Schwartz P J, Perry F, Ackerman M J, Laksman Z W M, Kaul P, Lieve K V V, Atallah J, Krahn A D, Sanatani S. Implantable cardioverter-defibrillator use in catecholaminergic polymorphic ventricular tachycardia: A systematic review. Heart Rhythm. 2018; 15:1791-1799.
11. Boule N G, Haddad E, Kenny G P, Wells G A, Sigal R J. Effects of exercise on glycemic control and body mass in type 2 diabetes mellitus: a meta-analysis of controlled clinical trials [Internet]. Scandinavian Journal of Medicine and Science in Sports. 2002; 12:60-61. Available from: http://dx.doi.org/10.1034/j.1600-0838.2002.120111_3.x
12. Garnvik L E, Malmo V, Janszky I, Ellekjær H, Wisløff U, Loennechen J P, Nes B M. Physical activity, cardiorespiratory fitness, and cardiovascular outcomes in individuals with atrial fibrillation: the HUNT study. Eur Heart J [Internet]. 2020; Available from: http://dx.doi.org/10.1093/eurheartj/ehaa032
13. Pedisic Z, Shrestha N, Kovalchik S, Stamatakis E, Liangruenrom N, Grgic J, Titze S, Biddle S J H, Bauman A E, Oja P. Is running associated with a lower risk of all-cause, cardiovascular and cancer mortality, and is the more the better? A systematic review and meta-analysis [Internet]. British Journal of Sports Medicine. 2019; bjsports-2018. Available from: http://dx.doi.org/10.1136/bjsports-2018-100493
14. Hayashi M, Denjoy I, Extramiana F, Maltret A, Buisson N R, LupoglazoffJ-M M, Klug D, Hayashi M, Takatsuki S, Villain E, Kamblock J, Messali A, Guicheney P, Lunardi J, Leenhardt A. Incidence and risk factors of arrhythmic events in catecholaminergic polymorphic ventricular tachycardia. Circulation. 2009; 119:2426-2434.
15. Kannankeril P J, Moore J P, Cerrone M, Priori S G, Kertesz N J, Ro P S, Batra A S, Kaufman E S, Fairbrother D L, Saarel E V, Etheridge S P, Kanter R J, Carboni M P, Dzurik M V, Fountain D, Chen H, Ely E W, Roden D M, Knollmann B C. Efficacy of Flecainide in the Treatment of Catecholaminergic Polymorphic Ventricular Tachycardia: A Randomized Clinical Trial. JAMA Cardiol. 2017; 2:759-766.
16. Echt D S, Liebson P R, Mitchell L B, Peters R W, Obias-Manno D, Barker A H, Arensberg D, Baker A, Friedman L, Greene H L. Mortality and morbidity in patients receiving encainide, flecainide, or placebo. The Cardiac Arrhythmia Suppression Trial. N Engl J Med. 1991; 324:781-788.
17. van der Werf C, Kannankeril P J, Sacher F, Krahn A D, Viskin S, Leenhardt A, Shimizu W, Sumitomo N, Fish F A, Bhuiyan Z A, Willems A R, van der Veen M J, Watanabe H, Laborderie J, Ha\“issaguerre M, Knollmann B C, Wilde A A M. Flecainide therapy reduces exercise-induced ventricular arrhythmias in patients with catecholaminergic polymorphic ventricular tachycardia. J Am Coll Cardiol. 2011; 57:2244-2254.
18. De Ferrari G M, Dusi V, Spazzolini C, Bos J M, Abrams D J, Berul C I, Crotti L, Davis A M, Eldar M, Kharlap M, Khoury A, Krahn A D, Leenhardt A, Moir C R, Odero A, Olde Nordkamp L, Paul T, Rosés I Noguer F, Shkolnikova M, Till J, Wilde A A M, Ackerman M J, Schwartz P J. Clinical Management of Catecholaminergic Polymorphic Ventricular Tachycardia: The Role of Left Cardiac Sympathetic Denervation. Circulation [Internet]. 2015; Available from: http://dx.doi.org/10.1161/CIRCULATIONAHA.115.015731
19. van der Werf C, Lieve K V, Bos J M, Lane C M, Denjoy I, Roses-Noguer F, Aiba T, Wada Y, Ingles J, Leren I S, Rudic B, Schwartz P J, Maltret A, Sacher F, Skinner J R, Krahn A D, Roston T M, Tfelt-Hansen J, Swan H, Robyns T, Ohno S, Roberts J D, van den Berg M P, Kammeraad J A, Probst V, Kannankeril P J, Blom N A, Behr E R, Borggrefe M, Haugaa K H, Semsarian C, Horie M, Shimizu W, Till J A, Leenhardt A, Ackerman M J, Wilde A A. Implantable cardioverter-defibrillators in previously undiagnosed patients with catecholaminergic polymorphic ventricular tachycardia resuscitated from sudden cardiac arrest. Eur Heart J [Internet]. 2019; Available from: http://dx.doi.org/10.1093/eurheartj/ehz309
20. Connell P, Word T A, Wehrens X H T. Targeting pathological leak of ryanodine receptors: preclinical progress and the potential impact on treatments for cardiac arrhythmias and heart failure. Expert Opin Ther Targets. 2020; 24:25-36.
21. Bezzerides V J, Caballero A, Wang S, Ai Y, Hylind R J, Lu F, Heims-Waldron D A, Chambers K D, Zhang D, Abrams D J, Pu W T. Gene Therapy for Catecholaminergic Polymorphic Ventricular Tachycardia by Inhibition of Ca²⁺/Calmodulin-Dependent Kinase II. Circulation. 2019; 140:405-419.
22. Stanczyk P J, Seidel M, White J, Viero C, George C H, Zissimopoulos S, Lai F A. Association of cardiac myosin-binding protein-C with the ryanodine receptor channel—putative retrograde regulation? J Cell Sci [Internet]. 2018; 131. Available from: http://dx.doi.org/10.1242/jcs.210443
23. Chelu M G, Sarma S, Sood S, Wang S, van Oort R J, Skapura D G, Li N, Santonastasi M, Müller F U, Schmitz W, Schotten U, Anderson M E, Valderrábano M, Dobrev D, Wehrens XHT. Calmodulin kinase II-mediated sarcoplasmic reticulum Ca²⁺ leak promotes atrial fibrillation in mice. J Clin Invest. 2009; 119:1940-1951.
24. Chelu M G, Sarma S, Sood S, Wang S, van Oort R J, Skapura D G, Li N, Santonastasi M, Müller F U, Schmitz W, Schotten U, Anderson M E, Valderrábano M, Dobrev D, Wehrens XHT. Calmodulin kinase II-mediated sarcoplasmic reticulum Ca²⁺ leak promotes atrial fibrillation in mice. J Clin Invest. 2009; 119:1940.
25. Denegri M, Bongianino R, Lodola F, Boncompagni S, De Giusti V C, Avelino-Cruz J E, Liu N, Persampieri S, Curcio A, Esposito F, Pietrangelo L, Marty I, Villani L, Moyaho A, Baiardi P, Auricchio A, Protasi F, Napolitano C, Priori S G. Single delivery of an adeno-associated viral construct to transfer the CASQ2 gene to knock-in mice affected by catecholaminergic polymorphic ventricular tachycardia is able to cure the disease from birth to advanced age. Circulation. 2014; 129:2673-2681.
26. Pellicena P, Schulman H. CaMKII inhibitors: from research tools to therapeutic agents. Front Pharmacol. 2014; 5:21.
27. Dadi P K, Vierra N C, Ustione A, Piston D W, Colbran R J, Jacobson D A. Inhibition of pancreatic β-cell Ca²⁺/calmodulin-dependent protein kinase II reduces glucose-stimulated calcium influx and insulin secretion, impairing glucose tolerance. J Biol Chem. 2014; 289:12435-12445.
28. Illario M, Monaco S, Cavallo A L, Esposito I, Formisano P, D'Andrea L, Cipolletta E, Trimarco B, Fenzi G, Rossi G, Vitale M. Calcium-calmodulin-dependent kinase II (CaMKII) mediates insulin-stimulated proliferation and glucose uptake. Cell Signal. 2009; 21:786-792.
29. Ozcan L, Cristina de Souza J, Harari A A, Backs J, Olson E N, Tabas I. Activation of calcium/calmodulin-dependent protein kinase II in obesity mediates suppression of hepatic insulin signaling. Cell Metab. 2013; 18:803-815.
30. Pan X, Philippen L, Lahiri S K, Lee C, Park S H, Word T A, Li N, Jarrett K E, Gupta R, Reynolds J O, Lin J, Bao G, Lagor W R, Wehrens XHT. In vivo Ryr2 Editing Corrects Catecholaminergic Polymorphic Ventricular Tachycardia. Circ Res. 2018; 123:953-963.
31. Bongianino R, Denegri M, Mazzanti A, Lodola F, Vollero A, Boncompagni S, Fasciano S, Rizzo G, Mangione D, Barbaro S, Di Fonso A, Napolitano C, Auricchio A, Protasi F, Priori S G. Allele Specific Silencing of Mutant mRNA Rescues Ultrastructural and Arrhythmic Phenotype in Mice Carriers of the R4496C Mutation in the Ryanodine Receptor Gene (RYR2). Circ Res [Internet]. 2017; Available from: http://dx.doi.org/10.1161/CIRCRESAHA.117.310882
32. Liu B, Walton S D, Ho H-T, Belevych A E, Tikunova S B, Bonilla I, Shettigar V, Knollmann B C, Priori S G, Volpe P, Radwański P B, Davis J P, Györke S. Gene Transfer of Engineered Calmodulin Alleviates Ventricular Arrhythmias in a Calsequestrin-Associated Mouse Model of Catecholaminergic Polymorphic Ventricular Tachycardia. J Am Heart Assoc [Internet]. 2018; 7. Available from: http://dx.doi.org/10.1161/JAHA.117.008155
33. Shannon T R, Ginsburg K S, Bers D M. Quantitative assessment of the SR Ca²⁺ leak-load relationship. Circ Res. 2002; 91:594-600.
34. Akerberg B N, Gu F, VanDusen N J, Zhang X, Dong R, Li K, Zhang B, Zhou B, Sethi I, Ma Q, Wasson L, Wen T, Liu J, Dong K, Conlon F L, Zhou J, Yuan G-C, Zhou P, Pu W T. A reference map of murine cardiac transcription factor chromatin occupancy identifies dynamic and conserved enhancers. Nat Commun. 2019; 10:4907.
35. Ellsworth J L, O'Callaghan M, Rubin H, Seymour A. Low Seroprevalence of Neutralizing Antibodies Targeting Two Clade F AAV in Humans. Hum Gene Ther Clin Dev. 2018; 29:60-67.
36. Boutin S, Monteilhet V, Veron P, Leborgne C, Benveniste O, Montus M F, Masurier C. Prevalence of serum IgG and neutralizing factors against adeno-associated virus (AAV) types 1, 2, 5, 6, 8, and 9 in the healthy population: implications for gene therapy using AAV vectors. Hum Gene Ther. 2010; 21:704-712.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

1. A method of treating a disorder associated with abnormal ryanodine receptor type 2 (RYR2) function, the method comprising administering to a subject in need thereof an effective amount of a polypeptide comprising a C-terminal domain of Cardiac Myosin binding protein C (MYBPC3).

2. A method of treating a disorder associated with abnormal ryanodine receptor type 2 (RYR2) function, the method comprising administering to a subject in need thereof an effective amount of a nucleic acid comprising a nucleotide sequence encoding a polypeptide comprising a C-terminal domain of Cardiac Myosin binding protein C (MYBPC3).

3. The method of claim 1, wherein the abnormal RYR2 function is caused by one or more mutations in RYR2.

4. The method of claim 3, wherein the mutation in RYR2 causes excessive diastolic Ca²⁺ release in cardiomyocytes in the subject.

5. The method of claim 1, wherein the polypeptide comprises an amino acid sequence that is at least 80% identical to any one of SEQ ID NOs: 1-16 or 53-64.

6.-7. (canceled)

8. The method of claim 2, wherein the nucleic acid is a vector.

9. The method of claim 8, wherein the vector is an expression vector.

10. The method of claim 9, wherein the expression vector is a viral vector.

11.-18. (canceled)

19. The method of claim 2, wherein the nucleic acid is a messenger RNA (mRNA).

20. (canceled)

21. The method of claim 1, wherein the polypeptide or the nucleic acid is delivered to a cardiomyocyte in the subject.

22. The method of claim 1, wherein the disorder is arrhythmia.

23.-27. (canceled)

28. The method of claim 1, wherein the disorder is heart failure.

29. The method of claim 25, wherein administering the polypeptide or the nucleic acid reduces the excessive diastolic Ca²⁺ release in cardiomyocytes in the subject.

30. The method of claim 1, wherein the subject is human.

31. The method of claim 1, wherein the administering is via injection.

32. A method of treating arrhythmia, the method comprising administering to a subject in need thereof an effective amount of a recombinant adeno-associated virus (rAAV), wherein the rAAV comprises a capsid protein of serotype AAV9 and a nucleotide sequence encoding a polypeptide comprising a C-terminal domain of Cardiac Myosin binding protein C (MYBPC3).

33. The method of claim 32 wherein the polypeptide comprises an amino acid sequence that is at least 80% identical to any one of SEQ ID NOs: 1-16 or 53-64.

34.-35. (canceled)

36. A recombinant adeno-associated virus (rAAV) comprising a capsid protein and a nucleotide sequence encoding a polypeptide comprising a C-terminal domain of Cardiac Myosin binding protein C (MYBPC3).

37. The rAAV of claim 33, wherein the polypeptide comprises the amino acid sequence of any one of SEQ ID NOs: 1-16 or 53-64.

38. (canceled)

39. Use of the rAAV of claim 33 in treating a disorder associated with abnormal ryanodine receptor type 2 (RYR2) function.
